(19) United States
(12) Patent Application Publication
(10) Pub. No.: US 2024/0265826 A1
(43) Pub. Date: Aug. 8, 2024

(54) Detecting Unhealthy Food and Using Tactile and/or Haptic Sensations to Reduce a Person's Consumption of the Unhealthy Food

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 18/617,950

(22) Filed: Mar. 27, 2024

Related U.S. Application Data
(63) Continuation-in-part of application No. 18/121,841, filed on Mar. 15, 2023.
Application No. 18/121,841 is a continuation-in-part of application No. 17/903,746, filed on Sep. 6, 2022, now abandoned.
Application No. 17/903,746 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/294,746, filed on Oct. 16, 2016, now Pat. No. 10,627,861.
Application No. 15/294,746 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/418,620, filed on Jan. 27, 2017, now abandoned.
Application No. 15/418,620 is a continuation-in-part of application No. 15/944,746, filed on Apr. 3, 2018, now abandoned.
Application No. 15/963,061 is a continuation-in-part of application No. 14/992,073, filed on Jan. 11, 2016, now abandoned.
Application No. 14/992,073 is a continuation-in-part of application No. 14/550,953, filed on Nov. 22, 2014, now abandoned.
Application No. 14/550,953 is a continuation-in-part of application No. 15/431,769, filed on Feb. 14, 2017, now abandoned.
Application No. 15/431,769 is a continuation-in-part of application No. 14/951,475, filed on Nov. 24, 2015, now Pat. No. 10,314,492.
Application No. 14/951,475 is a continuation-in-part of application No. 15/294,746, filed on Oct. 16, 2016, now Pat. No. 10,627,861.
Application No. 15/294,746 is a continuation-in-part of application No. 15/206,215, filed on Jul. 8, 2016, now abandoned.
Application No. 15/206,215 is a continuation-in-part of application No. 14/992,073, filed on Jan. 11, 2016, now abandoned.
Application No. 14/992,073 is a continuation-in-part of application No. 14/330,649, filed on Jul. 14, 2014, now abandoned.
Application No. 14/330,649 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/948,308, filed on Nov. 21, 2015, now abandoned.
Application No. 14/992,073 is a continuation-in-part of application No. 14/562,719, filed on Dec. 7, 2014, now Pat. No. 10,130,277.
Application No. 14/562,719 is a continuation-in-part of application No. 13/616,238, filed on Sep. 14, 2012, now abandoned.
Application No. 14/951,475 is a continuation-in-part of application No. 14/071,112, filed on Nov. 4, 2013, now abandoned.
Application No. 14/071,112 is a continuation-in-part of application No. 13/901,131, filed on May 23, 2013, now Pat. No. 9,536,449.
Application No. 14/948,308 is a continuation-in-part of application No. 14/550,953, filed on Nov. 22, 2014, now abandoned.
Application No. 14/550,953 is a continuation-in-part of application No. 14/449,387, filed on Aug. 1, 2014, now abandoned.
Application No. 14/449,387 is a continuation-in-part of application No. 14/132,292, filed on Dec. 18, 2013, now Pat. No. 9,442,100.
Application No. 14/132,292 is a continuation-in-part of application No. 13/901,099, filed on May 23, 2013, now Pat. No. 9,254,099.
Application No. 14/330,649 is a continuation-in-part of application No. 13/523,739, filed on Jun. 14, 2012, now Pat. No. 9,042,596.
(60) Provisional application No. 63/542,077, filed on Oct. 2, 2023.
(60) Provisional application No. 63/279,773, filed on Nov. 16, 2021.
(60) Provisional application No. 63/171,838, filed on Apr. 7, 2021.
(60) Provisional application No. 62/930,013, filed on Nov. 4, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/800,478, filed on Feb. 2, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/549,587, filed on Aug. 24, 2017.
(60) Provisional application No. 62/439,147, filed on Dec. 26, 2016.
(60) Provisional application No. 62/439,147, filed on Dec. 26, 2016.
(60) Provisional application No. 62/349,277, filed on Jun. 13, 2016.
(60) Provisional application No. 62/311,462, filed on Mar. 22, 2016.
(60) Provisional application No. 62/297,827, filed on Feb. 20, 2016.
(60) Provisional application No. 62/349,277, filed on Jun. 13, 2016.
(60) Provisional application No. 62/245,311, filed on Oct. 23, 2015.
(60) Provisional application No. 62/349,277, filed on Jun. 13, 2016.
(60) Provisional application No. 61/932,517, filed on Jan. 28, 2014.

(51) Int. Cl. G09B 19/00; G06F 3/01; G06V 20/68; G16H 20/60; H04N 23/50

(52) U.S. Cl. CPC G09B 19/0092; G06F 3/016; G06V 20/68; G16H 20/60; H04N 23/50

(57) ABSTRACT

This invention can be embodied in methods and systems for reducing a person's consumption of unhealthy food by providing the person with haptic and/or tactile sensations in response to detection of unhealthy food near the person and/or detection of consumption of unhealthy food by the person. Detection of unhealthy food near the person can be done using a camera on a device worn by the person. Detection of consumption of unhealthy food by the person can be done using a food consumption sensor on a device worn by the person. Haptic and/or tactile sensations can be vibrations.

### CROSS-REFERENCE TO RELATED APPLICATIONS

This application claims the priority benefit of U.S. provisional application 63/542,077 filed on 2023 Oct. 2. This application is a continuation-in-part of U.S. patent application Ser. No. 18/121,841 filed on 2023 Mar. 15.

U.S. patent application Ser. No. 18/121,841 was a continuation-in-part of U.S. patent application Ser. No. 17/903,746 filed on 2022 Sep. 6. U.S. patent application Ser. No. 18/121,841 was a continuation-in-part of U.S. patent application Ser. No. 17/239,960 filed on 2021 Apr. 26. U.S. patent application Ser. No. 18/121,841 was a continuation-in-part of U.S. patent application Ser. No. 16/737,052 filed on 2020 Jan. 8.

U.S. patent application Ser. No. 17/903,746 was a continuation-in-part of U.S. patent application Ser. No. 16/568,580 filed on 2019 Sep. 12. U.S. patent application Ser. No. 17/903,746 was a continuation-in-part of U.S. patent application Ser. No. 16/737,052 filed on 2020 Jan. 8. U.S. patent application Ser. No. 17/903,746 was a continuation-in-part of U.S. patent application Ser. No. 17/239,960 filed on 2021 Apr. 26. U.S. patent application Ser. No. 17/903,746 claimed the priority benefit of U.S. provisional application 63/279,773 filed on 2021 Nov. 16.

U.S. patent application Ser. No. 17/239,960 claimed the priority benefit of U.S. provisional application 63/171,838 filed on 2021 Apr. 7. U.S. patent application Ser. No. 17/239,960 was a continuation-in-part of U.S. patent application Ser. No. 16/737,052 filed on 2020 Jan. 8.

U.S. patent application Ser. No. 16/737,052 claimed the priority benefit of U.S. provisional application 62/930,013 filed on 2019 Nov. 4. U.S. patent application Ser. No. 16/737,052 claimed the priority benefit of U.S. provisional application 62/857,942 filed on 2019 Jun. 6. U.S. patent application Ser. No. 16/737,052 claimed the priority benefit of U.S. provisional application 62/814,713 filed on 2019 Mar. 6. U.S. patent application Ser. No. 16/737,052 claimed the priority benefit of U.S. provisional application 62/814,692 filed on 2019 Mar. 6. U.S. patent application Ser. No. 16/737,052 claimed the priority benefit of U.S. provisional application 62/800,478 filed on 2019 Feb. 2. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25 which issued as U.S. patent Ser. No. 10/772,559 on 2020 Sep. 15. U.S. patent application Ser. No. 16/737,052 was a continuation-in-part of U.S. patent application Ser. No. 15/725,330 filed on 2017 Oct. 5 which issued as U.S. patent Ser. No. 10/607,507 on 2020 Mar. 31. U.S. patent application Ser. No. 16/737,052 was a continuation-in-part of U.S. patent application Ser. No. 15/431,769 filed on 2017 Feb. 14. U.S. patent application Ser. No. 16/737,052 was a continuation-in-part of U.S. patent application Ser. No. 15/294,746 filed on 2016 Oct. 16 which issued as U.S. patent Ser. No. 10/627,861 on 2020 Apr. 21.

U.S. patent application Ser. No. 16/568,580 claimed the priority benefit of U.S. provisional application 62/857,942 filed on 2019 Jun. 6. U.S. patent application Ser. No. 16/568,580 claimed the priority benefit of U.S. provisional application 62/814,713 filed on 2019 Mar. 6. U.S. patent application Ser. No. 16/568,580 claimed the priority benefit of U.S. provisional application 62/814,692 filed on 2019 Mar. 6. U.S. patent application Ser. No. 16/568,580 was a continuation-in-part of U.S. patent application Ser. No. 15/963,061 filed on 2018 Apr. 25 which issued as U.S. patent Ser. No. 10/772,559 on 2020 Sep. 15. U.S. patent application Ser. No. 16/568,580 was a continuation-in-part of U.S. patent application Ser. No. 15/725,330 filed on 2017 Oct. 5 which issued as U.S. patent Ser. No. 10/607,507 on 2020 Mar. 31. U.S. patent application Ser. No. 16/568,580 was a continuation-in-part of U.S. patent application Ser. No. 15/431,769 filed on 2017 Feb. 14. U.S. patent application Ser. No. 16/568,580 was a continuation-in-part of U.S. patent application Ser. No. 15/418,620 filed on 2017 Jan. 27. U.S. patent application Ser. No. 16/568,580 was a continuation-in-part of U.S. patent application Ser. No. 15/294,746 filed on 2016 Oct. 16 which issued as U.S. patent Ser. No. 10/627,861 on 2020 Apr. 21.

U.S. patent application Ser. No. 15/963,061 was a continuation-in-part of U.S. patent application Ser. No. 14/992,073 filed on 2016 Jan. 11. U.S. patent application Ser. No. 15/963,061 was a continuation-in-part of U.S. patent application Ser. No. 14/550,953 filed on 2014 Nov. 22.

U.S. patent application Ser. No. 15/725,330 claimed the priority benefit of U.S. provisional application 62/549,587 filed on 2017 Aug. 24. U.S. patent application Ser. No. 15/725,330 claimed the priority benefit of U.S. provisional application 62/439,147 filed on 2016 Dec. 26. U.S. patent application Ser. No. 15/725,330 was a continuation-in-part of U.S. patent application Ser. No. 15/431,769 filed on 2017 Feb. 14. U.S. patent application Ser. No. 15/725,330 was a continuation-in-part of U.S. patent application Ser. No. 14/951,475 filed on 2015 Nov. 24 which issued as U.S. patent Ser. No. 10/314,492 on 2019 Jun. 11.

U.S. patent application Ser. No. 15/431,769 claimed the priority benefit of U.S. provisional application 62/439,147 filed on 2016 Dec. 26. U.S. patent application Ser. No. 15/431,769 claimed the priority benefit of U.S. provisional application 62/349,277 filed on 2016 Jun. 13. U.S. patent application Ser. No. 15/431,769 claimed the priority benefit of U.S. provisional application 62/311,462 filed on 2016 Mar. 22. U.S. patent application Ser. No. 15/431,769 was a continuation-in-part of U.S. patent application Ser. No. 15/294,746 filed on 2016 Oct. 16 which issued as U.S. patent Ser. No. 10/627,861 on 2020 Apr. 21. U.S. patent application Ser. No. 15/431,769 was a continuation-in-part of U.S. patent application Ser. No. 15/206,215 filed on 2016 Jul. 8. U.S. patent application Ser. No. 15/431,769 was a continuation-in-part of U.S. patent application Ser. No. 14/992,073 filed on 2016 Jan. 11. U.S. patent application Ser. No. 15/431,769 was a continuation-in-part of U.S. patent application Ser. No. 14/330,649 filed on 2014 Jul. 14.

U.S. patent application Ser. No. 15/418,620 claimed the priority benefit of U.S. provisional application 62/297,827 filed on 2016 Feb. 20. U.S. patent application Ser. No. 15/418,620 was a continuation-in-part of U.S. patent application Ser. No. 14/951,475 filed on 2015 Nov. 24 which issued as U.S. patent Ser. No. 10/314,492 on 2019 Jun. 11.

U.S. patent application Ser. No. 15/294,746 claimed the priority benefit of U.S. provisional application 62/349,277 filed on 2016 Jun. 13. U.S. patent application Ser. No. 15/294,746 claimed the priority benefit of U.S. provisional application 62/245,311 filed on 2015 Oct. 23. U.S. patent application Ser. No. 15/294,746 was a continuation-in-part of U.S. patent application Ser. No. 14/951,475 filed on 2015 Nov. 24 which issued as U.S. patent Ser. No. 10/314,492 on 2019 Jun. 11.

U.S. patent application Ser. No. 15/206,215 claimed the priority benefit of U.S. provisional application 62/349,277 filed on 2016 Jun. 13. U.S. patent application Ser. No. 15/206,215 was a continuation-in-part of U.S. patent application Ser. No. 14/951,475 filed on 2015 Nov. 24 which issued as U.S. patent Ser. No. 10/314,492 on 2019 Jun. 11. U.S. patent application Ser. No. 15/206,215 was a continuation-in-part of U.S. patent application Ser. No. 14/948,308 filed on 2015 Nov. 21.

U.S. patent application Ser. No. 14/992,073 was a continuation-in-part of U.S. patent application Ser. No. 14/562,719 filed on 2014 Dec. 7 which issued as U.S. patent Ser. No. 10/130,277 on 2018 Nov. 20. U.S. patent application Ser. No. 14/992,073 was a continuation-in-part of U.S. patent application Ser. No. 13/616,238 filed on 2012 Sep. 14.

U.S. patent application Ser. No. 14/951,475 was a continuation-in-part of U.S. patent application Ser. No. 14/071,112 filed on 2013 Nov. 4. U.S. patent application Ser. No. 14/951,475 was a continuation-in-part of U.S. patent application Ser. No. 13/901,131 filed on 2013 May 23 which issued as U.S. Pat. No. 9,536,449 on 2017 Jan. 3.

U.S. patent application Ser. No. 14/948,308 was a continuation-in-part of U.S. patent application Ser. No. 14/550,953 filed on 2014 Nov. 22. U.S. patent application Ser. No. 14/948,308 was a continuation-in-part of U.S. patent application Ser. No. 14/449,387 filed on 2014 Aug. 1. U.S. patent application Ser. No. 14/948,308 was a continuation-in-part of U.S. patent application Ser. No. 14/132,292 filed on 2013 Dec. 18 which issued as U.S. Pat. No. 9,442,100 on 2016 Sep. 13. U.S. patent application Ser. No. 14/948,308 was a continuation-in-part of U.S. patent application Ser. No. 13/901,099 filed on 2013 May 23 which issued as U.S. Pat. No. 9,254,099 on 2016 Feb. 9.

U.S. patent application Ser. No. 14/562,719 claimed the priority benefit of U.S. provisional application 61/932,517 filed on 2014 Jan. 28.

U.S. patent application Ser. No. 14/330,649 was a continuation-in-part of U.S. patent application Ser. No. 13/523,739 filed on 2012 Jun. 14 which issued as U.S. Pat. No. 9,042,596 on 2015 May 26.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable devices for reducing consumption of unhealthy food.

### INTRODUCTION

Many health problems are caused by poor nutrition, including consumption of too much unhealthy food. There are complex behavioral reasons for poor eating habits. Although not a panacea, real time monitoring and modification of a person's food consumption (e.g. to reduce consumption of unhealthy amounts and/or types of food) can help the person to improve their eating habits and health.

### REVIEW OF THE RELEVANT ART

In the patent literature, U.S. patent Ser. No. 10/901,509 (Aimone et al., Jan. 26, 2021, “Wearable Computing Apparatus and Method”) discloses a wearable computing device comprising at least one brainwave sensor. U.S. patent Ser. No. 11/222,422 (Alshurafa et al., Jan. 11, 2022, “Hyperspectral Imaging Sensor”) discloses a hyperspectral imaging sensor system to identify item composition. U.S. patent application 20160148535 (Ashby, May 26, 2016, “Tracking Nutritional Information about Consumed Food”) discloses an eating monitor which monitors swallowing and/or chewing. U.S. patent application 20160148536 (Ashby, May 26, 2016, “Tracking Nutritional Information about Consumed Food with a Wearable Device”) discloses an eating monitor with a camera. U.S. Pat. No. 9,146,147 (Bakhsh, Sep. 29, 2015, “Dynamic Nutrition Tracking Utensils”) discloses nutritional intake tracking using a smart utensil.

U.S. patent application 20210307677 (Bi et al., Oct. 7, 2021, “System for Detecting Eating with Sensor Mounted by the Ear”) discloses a wearable device for detecting eating episodes via a contact microphone. U.S. patent application 20210307686 (Catani et al., Oct. 7, 2021, “Methods and Systems to Detect Eating”) discloses methods and systems for automated eating detection comprising a continuous glucose monitor (CGM) and an accelerometer. U.S. patent application 20190213416 (Cho et al., Jul. 11, 2019, “Electronic Device and Method for Processing Information Associated with Food”) and patent Ser. No. 10/803,315 (Cho et al., Oct. 13, 2020, “Electronic Device and Method for Processing Information Associated with Food”) disclose analysis of food images to obtain nutritional information concerning food items and to recommend food consumption quantities. U.S. patent application 20170061821 (Choi et al., Mar. 2, 2017, “Systems and Methods for Performing a Food Tracking Service for Tracking Consumption of Food Items”) discloses a food tracking service. U.S. patent application 20190167190 (Choi et al., Jun. 6, 2019, “Healthcare Apparatus and Operating Method Thereof”) discloses a dietary monitoring device which emits light of different wavelengths.

U.S. patent application Ser. No. 11/478,096 (Chung et al., Oct. 25, 2022, “Food Monitoring System”) discloses a serving receptacle, an information processor, and utensils which are used to estimate food quantity. U.S. patent application 20220400195 (Churovich et al., Dec. 15, 2022, “Electronic Visual Food Probe”) and patent Ser. No. 11/366,305 (Churovich et al., Jun. 21, 2022, “Electronic Visual Food Probe”) disclose an electronic visual food probe to view inside food. U.S. patent Ser. No. 10/143,420 (Contant, Dec. 4, 2018, “Eating Utensil to Monitor and Regulate Dietary Intake”) discloses a dietary intake regulating device that also monitors physical activity. U.S. patent application 20160163037 (Dehais et al., Jun. 9, 2016, “Estimation of Food Volume and Carbs”) discloses an image-based food identification system including a projected light pattern. U.S. patent application 20170249445 (Devries et al., Aug. 31, 2017, “Portable Devices and Methods for Measuring Nutritional Intake”) discloses a nutritional intake monitoring system with biosensors.

U.S. patent application 20180214077 (Dunki-Jacobs, Aug. 2, 2018, “Meal Detection Devices and Methods”) and patent Ser. No. 10/791,988 (Dunki-Jacobs, Aug. 2, 2018, “Meal Detection Devices and Methods”) disclose using biometric sensors to detect meal intake and control a therapeutic device. U.S. patent application 20150294450 (Eyring, Oct. 15, 2015, “Systems and Methods for Measuring Calorie Intake”) discloses an image-based system for measuring caloric input. U.S. patent application 20220301683 (Feilner, Sep. 22, 2022, “Detecting and Quantifying a Liquid and/or Food Intake of a User Wearing a Hearing Device”) discloses detecting and quantifying a food intake via a microphone.

U.S. patent applications 20090012433 (Fernstrom et al., Jan. 8, 2009, “Method, Apparatus and System for Food Intake and Physical Activity Assessment”), 20130267794 (Fernstrom et al., Oct. 10, 2013, “Method, Apparatus and System for Food Intake and Physical Activity Assessment”), and 20180348187 (Fernstrom et al., Dec. 6, 2018, “Method, Apparatus and System for Food Intake and Physical Activity Assessment”), as well as U.S. Pat. No. 9,198,621 (Fernstrom et al., Dec. 1, 2015, “Method, Apparatus and System for Food Intake and Physical Activity Assessment”) and 10006896 (Fernstrom et al., Jun. 26, 2018, “Method, Apparatus and System for Food Intake and Physical Activity Assessment”), disclose wearable buttons and necklaces for monitoring eating with cameras. U.S. patent Ser. No. 10/900,943 (Fernstrom et al, Jan. 26, 2021, “Method, Apparatus and System for Food Intake and Physical Activity Assessment”) discloses monitoring food consumption using a wearable device with two video cameras and an infrared sensor. U.S. patent application 20150325142 (Ghalavand, Nov. 12, 2015, “Calorie Balance System”) discloses a calorie balance system with smart utensils and/or food scales.

U.S. patent applications 20160299061 (Goldring et al., Oct. 13, 2016, “Spectrometry Systems, Methods, and Applications”), 20170160131 (Goldring et al., Jun. 8, 2017, “Spectrometry Systems, Methods, and Applications”), 20180085003 (Goldring et al., Mar. 29, 2018, “Spectrometry Systems, Methods, and Applications”), 20180120155 (Rosen et al., May 3, 2018, “Spectrometry Systems, Methods, and Applications”), and 20180180478 (Goldring et al., Jun. 28, 2018, “Spectrometry Systems, Methods, and Applications”) disclose a handheld spectrometer to measure the spectra of objects. U.S. patent application 20180136042 (Goldring et al., May 17, 2018, “Spectrometry System with Visible Aiming Beam”) discloses a handheld spectrometer with a visible aiming beam. U.S. patent application 20180252580 (Goldring et al., Sep. 6, 2018, “Low-Cost Spectrometry System for End-User Food Analysis”) discloses a compact spectrometer that can be used in mobile devices such as smart phones. U.S. patent application 20190033130 (Goldring et al., Jan. 31, 2019, “Spectrometry Systems, Methods, and Applications”) discloses a hand held spectrometer with wavelength multiplexing. U.S. patent application 20190033132 (Goldring et al., Jan. 31, 2019, “Spectrometry System with Decreased Light Path”) discloses a spectrometer with a plurality of isolated optical channels. U.S. patent application 20190041265 (Rosen et al., Feb. 7, 2019, “Spatially Variable Filter Systems and Methods”) discloses a compact spectrometer system with a spatially variable filter.

U.S. patent application 20190295440 (Hadad, Sep. 26, 2019, “Systems and Methods for Food Analysis, Personalized Recommendations and Health Management”) discloses a method for developing a food ontology. U.S. patent applications 20190244541 (Hadad et al., Aug. 8, 2019, “Systems and Methods for Generating Personalized Nutritional Recommendations”), 20140255882 (Hadad et al., Sep. 11, 2014, “Interactive Engine to Provide Personal Recommendations for Nutrition, to Help the General Public to Live a Balanced Healthier Lifestyle”), and 20190290172 (Hadad et al., Sep. 26, 2019, “Systems and Methods for Food Analysis, Personalized Recommendations, and Health Management”) disclose methods to provide nutrition recommendations based on a person's preferences, habits, medical and activity.

U.S. patent application 20190272845 (Hasan et al., Sep. 5, 2019, “System and Method for Monitoring Dietary Activity”) discloses a system for monitoring dietary activity via a neck-worn device with an audio input unit. U.S. patent application 20160103910 (Kim et al., Apr. 14, 2016, “System and Method for Food Categorization”) discloses a food categorization engine. U.S. patent application 20190244704 (Kim et al., Aug. 8, 2019, “Dietary Habit Management Apparatus and Method”) discloses a dietary habit management apparatus using biometric measurements. U.S. patent application 20200015697 (Kinreich, Apr. 15, 2021, “Method and System for Analyzing Neural and Muscle Activity in a Subject's Head for the Detection of Mastication”) discloses a wearable apparatus to automatically monitor consumption by analyzing images captured of the environment. U.S. patent application 20220012467 (Kuo et al., Jan. 13, 2022, “Multi-Sensor Analysis of Food”) discloses a method for estimating food composition by 3D imaging and millimeter-wave radar.

U.S. patent application 20160140869 (Kuwahara et al., May 19, 2016, “Food Intake Controlling Devices and Methods”) discloses image-based technologies for controlling food intake. U.S. patent Ser. No. 10/359,381 (Lewis et al., Jul. 23, 2019, “Methods and Systems for Determining an Internal Property of a Food Product”) and U.S. Pat. No. 11,313,820 (Lewis et al., Apr. 26, 2022, “Methods and Systems for Determining an Internal Property of a Food Product”) disclose analyzing interior and external properties of food. U.S. patent application 20170156634 (Li et al., Jun. 8, 2017, “Wearable Device and Method for Monitoring Eating”) and patent Ser. No. 10/499,833 (Li et al., Dec. 10, 2019, “Wearable Device and Method for Monitoring Eating”) disclose a wearable device with an acceleration sensor to monitor eating. U.S. patent Ser. No. 11/568,760 (Meier, Jan. 31, 2023, “Augmented Reality Calorie Counter”) discloses using chewing noises and food images to estimate food volume. U.S. patent Ser. No. 10/952,670 (Mori et al., Mar. 23, 2021, “Meal Detection Method, Meal Detection System, and Storage Medium”) discloses meal detection by analyzing arm motion data and heart rate data.

U.S. patent application 20150302160 (Muthukumar et al., Oct. 22, 2015, “Method and Apparatus for Monitoring Diet and Activity”) discloses a method and device for analyzing food with a camera and a spectroscopic sensor. U.S. patent Ser. No. 10/249,214 (Novotny et al., Apr. 2, 2019, “Personal Wellness Monitoring System”) and U.S. Pat. No. 11,206,980 (Novotny et al., Dec. 28, 2021, “Personal Wellness Monitoring System”) disclose a personal nutrition, health, wellness and fitness monitor that captures 3D images. U.S. patent application 20160313241 (Ochi et al., Nov. 27, 2016, “Calorie Measurement Device”) disclose, Mar. 17, 2016, “Food Intake Monitor”) discloses a jaw motion sensor to measure food intake. U.S. patent application 20210183493 (Oh et al., Jun. 17, 2021, “Systems and Methods for Automatic Activity Tracking”) discloses systems and methods for tracking activities (e.g., eating moments) from a plurality of multimodal inputs.

U.S. Pat. No. 9,349,297 (Ortiz et al., May 24, 2016, “System and Method for Nutrition Analysis Using Food Image Recognition”) discloses a system and method for determining the nutritional value of a food item. U.S. Pat. No. 9,364,106 (Ortiz, Jun. 14, 2016, “Apparatus and Method for Identifying, Measuring and Analyzing Food Nutritional Values and Consumer Eating Behaviors”) discloses a food container for determining the nutritional value of a food item. U.S. patent application 20180005545 (Pathak et al., Jan. 4, 2018, “Assessment of Nutrition Intake Using a Handheld Tool”) discloses a smart food utensil for measuring food mass. U.S. patent application 20210369187 (Raju et al., Dec. 2, 2021, “Non-Contact Chewing Sensor and Portion Estimator”) discloses an optical proximity sensor to monitor chewing. U.S. patent Ser. No. 10/423,045 (Roberts et al., Sep. 24, 2019, “Electro-Optical Diffractive Waveplate Beam Shaping System”) discloses optical beam shaping systems with a diffractive waveplate diffuser.

U.S. patent application 20160073953 (Sazonov et al., Mar. 17, 2016, “Food Intake Monitor”) discloses monitoring food consumption using a wearable device with a jaw motion sensor and a hand gesture sensor. U.S. patent application 20180242908 (Sazonov et al., Aug. 30, 2018, “Food Intake Monitor”) and U.S. patent Ser. No. 10/736,566 (Sazonov, Aug. 11, 2020, “Food Intake Monitor”) disclose monitoring food consumption using an ear-worn device or eyeglasses with a pressure sensor and accelerometer. U.S. patent applications 20200337635 (Sazonov et al., Oct. 29, 2020, “Food Intake Monitor”) and 20210345959 (Sazonov et al., Nov. 11, 2021, “Food Intake Monitor”) and U.S. patent Ser. No. 11/006,896 (Sazonov et al., May 18, 2021, “Food Intake Monitor”) and U.S. Pat. No. 11,564,623 (Sazonov et al., Jan. 31, 2023, “Food Intake Monitor”) disclose an optical proximity sensor and/or temporalis muscle activity sensor to monitor chewing.

U.S. patent application 20210110159 (Shashua et al., Apr. 15, 2021, “Systems and Methods for Monitoring Consumption”) and patent Ser. No. 11/462,006 (Shashua et al., Oct. 4, 2022, “Systems and Methods for Monitoring Consumption”) disclose a wearable apparatus to automatically monitor consumption by a user by analyzing images. U.S. patent Ser. No. 10/952,669 (Shi et al., Mar. 23, 2021, “System for Monitoring Eating Habit Using a Wearable Device”) discloses a wearable device for monitoring eating behavior with an imaging sensor and an electromyography (EMG) sensor. U.S. patent Ser. No. 11/510,610 (Tanimura et al., Nov. 29, 2022, “Eating Monitoring Method, Program, and Eating Monitoring Device”) discloses an eating monitoring method using a sensor to measure jaw movement. U.S. patent Ser. No. 11/013,430 (Tanriover et al., May 25, 2021, “Methods and Apparatus for Identifying Food Chewed and/or Beverage Drank”) discloses methods and apparatuses for identifying food consumption via a chewing analyzer that extracts vibration data.

U.S. patent application 20190333634 (Vleugels et al., Oct. 31, 2019, “Method and Apparatus for Tracking of Food Intake and Other Behaviors and Providing Relevant Feedback”), 20170220772 (Vleugels et al., Aug. 3, 2017, “Method and Apparatus for Tracking of Food Intake and Other Behaviors and Providing Relevant Feedback”), and 20180300458 (Vleugels et al., Oct. 18, 2018, “Method and Apparatus for Tracking of Food Intake and Other Behaviors and Providing Relevant Feedback”), as well as U.S. patent Ser. No. 10/102,342 (Vleugels et al., Oct. 16, 2018, “Method and Apparatus for Tracking of Food Intake and Other Behaviors and Providing Relevant Feedback”) and 10373716 (Vleugels et al., Aug. 6, 2019, “Method and Apparatus for Tracking of Food Intake and Other Behaviors and Providing Relevant Feedback”), disclose a method for detecting, identifying, analyzing, quantifying, tracking, processing and/or influencing food consumption. U.S. patent application 20190236465 (Vleugels, Aug. 1, 2019, “Activation of Ancillary Sensor Systems Based on Triggers from a Wearable Gesture Sensing Device”) discloses an eating monitor with gesture recognition.

U.S. patent application 20200294645 (Vleugels, Sep. 17, 2020, “Gesture-Based Detection of a Physical Behavior Event Based on Gesture Sensor Data and Supplemental Information from at Least One External Source”) discloses an automated medication dispensing system which recognizes gestures. U.S. patent Ser. No. 10/790,054 (Vleugels et al., Sep. 29, 2020, “Method and Apparatus for Tracking of Food Intake and Other Behaviors and Providing Relevant Feedback”) discloses a computer-based method of detecting gestures. U.S. patent application 20200381101 (Vleugels, Dec. 3, 2020, “Method and Apparatus for Tracking of Food Intake and Other Behaviors and Providing Relevant Feedback”) discloses methods for detecting, identifying, analyzing, quantifying, tracking, processing and/or influencing, related to the intake of food, eating habits, eating patterns, and/or triggers for food intake events, eating habits, or eating patterns.

U.S. patent applications 20200381101 (Vleugels, Dec. 3, 2020, “Method and Apparatus for Tracking of Food Intake and Other Behaviors and Providing Relevant Feedback”) and 20210350920 (Vleugels et al., Nov. 11, 2021, “Method and Apparatus for Tracking of Food Intake and Other Behaviors and Providing Relevant Feedback”) disclose methods for detecting, identifying, analyzing, quantifying, tracking, processing and/or influencing the intake of food, eating habits, eating patterns, and/or triggers for food intake events, eating habits, or eating patterns.

U.S. patent application 20160091419 (Watson et al., Mar. 31, 2016, “Analyzing and Correlating Spectra, Identifying Samples and Their Ingredients, and Displaying Related Personalized Information”) discloses a spectral analysis method for food analysis. U.S. patent applications 20170292908 (Wilk et al., Oct. 12, 2017, “Spectrometry System Applications”) and 20180143073 (Goldring et al., May 24, 2018, “Spectrometry System Applications”) disclose a spectrometer system to determine spectra of an object. U.S. patent application 20170193854 (Yuan et al., Jan. 5, 2016, “Smart Wearable Device and Health Monitoring Method”) discloses a wearable device with a camera to monitor eating. U.S. patent Ser. No. 10/058,283 (Zerick et al., Apr. 6, 2016, “Determining Food Identities with Intra-Oral Spectrometer Devices”) discloses an intra-oral device for food analysis.

In the non-patent literature, Amft et al., 2005 (“Detection of Eating and Drinking Arm Gestures Using Inertial Body-Worn Sensors”) discloses eating detection by analyzing arm gestures. Bedri et al., 2015 (“Detecting Mastication: A Wearable Approach”; access to abstract only) discloses eating detection using an ear-worn devices with a gyroscope and proximity sensors. Bedri et al., 2017 (“EarBit: Using Wearable Sensors to Detect Eating Episodes in Unconstrained Environments”) discloses eating detection using an ear-worn device with inertial, optical, and acoustic sensors. Bedri et al., 2020a (“FitByte: Automatic Diet Monitoring in Unconstrained Situations Using Multimodal Sensing on Eyeglasses”) discloses food consumption monitoring using a device with a motion sensor, an infrared sensor, and a camera which is attached to eyeglasses. Bell et al., 2020 (“Automatic, Wearable-Based, In-Field Eating Detection Approaches for Public Health Research: A Scoping Review”) reviews wearable sensors for eating detection.

Bi et al., 2016 (“AutoDietary: A Wearable Acoustic Sensor System for Food Intake Recognition in Daily Life”) discloses eating detection using a neck-worn device with sound sensors. Bi et al., 2017 (“Toward a Wearable Sensor for Eating Detection”) discloses eating detection using ear-worn and neck-worn devices with sound sensors and EMG sensors. Bi et al., 2018 (“Auracle: Detecting Eating Episodes with an Ear-Mounted Sensor”) discloses eating detection using an ear-worn device with a microphone. Borrell, 2011 (“Every Bite You Take”) discloses food consumption monitoring using a neck-worn device with GPS, a microphone, an accelerometer, and a camera. Brenna et al., 2019 (“A Survey of Automatic Methods for Nutritional Assessment) reviews automatic methods for nutritional assessment. Chun et al., 2018 (“Detecting Eating Episodes by Tracking Jawbone Movements with a Non-Contact Wearable Sensor”) discloses eating detection using a necklace with an accelerometer and range sensor.

Chung et al., 2017 (“A Glasses-Type Wearable Device for Monitoring the Patterns of Food Intake and Facial Activity”) discloses eating detection using a force-based chewing sensor on eyeglasses. Dimitratos et al., 2020 (“Wearable Technology to Quantify the Nutritional Intake of Adults: Validation Study”) discloses high variability in food consumption monitoring using only a wristband with a motion sensor. Dong et al., 2009 (“A Device for Detecting and Counting Bites of Food Taken by a Person During Eating”) discloses bite counting using a wrist-worn orientation sensor. Dong et al., 2011 (“Detecting Eating Using a Wrist Mounted Device During Normal Daily Activities”) discloses eating detection using a watch with a motion sensor. Dong et al., 2012b (“A New Method for Measuring Meal Intake in Humans via Automated Wrist Motion Tracking”) discloses bite counting using a wrist-worn gyroscope. Dong et al., 2014 (“Detecting Periods of Eating During Free-Living by Tracking Wrist Motion”) discloses eating detection using a wrist-worn device with motion sensors.

Farooq et al., 2016 (“A Novel Wearable Device for Food Intake and Physical Activity Recognition”) discloses eating detection using eyeglasses with a piezoelectric strain sensor and an accelerometer. Farooq et al., 2017 (“Segmentation and Characterization of Chewing Bouts by Monitoring Temporalis Muscle Using Smart Glasses With Piezoelectric Sensor”) discloses chew counting using eyeglasses with a piezoelectric strain sensor. Fontana et al., 2014 (“Automatic Ingestion Monitor: A Novel Wearable Device for Monitoring of Ingestive Behavior”) discloses food consumption monitoring using a device with a jaw motion sensor, a hand gesture sensor, and an accelerometer. Fontana et al., 2015 (“Energy Intake Estimation from Counts of Chews and Swallows”) discloses counting chews and swallows using wearable sensors and video analysis. Jasper et al., 2016 (“Effects of Bite Count Feedback from a Wearable Device and Goal-Setting on Consumption in Young Adults”) discloses the effect of feedback based on bite counting.

Liu et al., 2012 (“An Intelligent Food-Intake Monitoring System Using Wearable Sensors”) discloses food consumption monitoring using an ear-worn device with a microphone and camera. Magrini et al., 2017 (“Wearable Devices for Caloric Intake Assessment: State of Art and Future Developments”) reviews wearable devices for automatic recording of food consumption. Makeyev et al., 2012 (“Automatic Food Intake Detection Based on Swallowing Sounds”) discloses swallowing detection using wearable sound sensors. Merck et al., 2016 (“Multimodality Sensing for Eating Recognition”; access to abstract only) discloses eating detection using eyeglasses and smart watches on each wrist, combining motion and sound sensors.

Mirtchouk et al., 2016 (“Automated Estimation of Food Type and Amount Consumed from Body-Worn Audio and Motion Sensors”; access to abstract only) discloses food consumption monitoring using in-ear audio plus head and wrist motion. Mirtchouk et al., 2017 (“Recognizing Eating from Body-Worn Sensors: Combining Free-Living and Laboratory Data”) discloses eating detection using head-worn and wrist-worn motion sensors and sound sensors. O'Loughlin et al., 2013 (“Using a Wearable Camera to Increase the Accuracy of Dietary Analysis”) discloses food consumption monitoring using a combination of a wearable camera and self-reported logging. Prioleau et al., 2017 (“Unobtrusive and Wearable Systems for Automatic Dietary Monitoring”) reviews wearable and hand-held approaches to dietary monitoring. Rahman et al., 2015 (“Unintrusive Eating Recognition Using Google Glass”) discloses eating detection using eyeglasses with an inertial motion sensor.

Sazonov et al., 2008 (“Non-Invasive Monitoring of Chewing and Swallowing for Objective Quantification of Ingestive Behavior”) discloses counting chews and swallows using ear-worn and/or neck-worn strain and sound sensors. Sazonov et al., 2009 (“Toward Objective Monitoring of Ingestive Behavior in Free-Living Population”) discloses counting chews and swallows using strain sensors. Sazonov et al., 2010a (“The Energetics of Obesity: A Review: Monitoring Energy Intake and Energy Expenditure in Humans”) reviews devices for monitoring food consumption. Sazonov et al., 2010b (“Automatic Detection of Swallowing Events by Acoustical Means for Applications of Monitoring of Ingestive Behavior”) discloses swallowing detection using wearable sound sensors. Sazonov et al., 2012 (“A Sensor System for Automatic Detection of Food Intake Through Non-Invasive Monitoring of Chewing”) discloses eating detection using a wearable piezoelectric strain gauge.

Schiboni et al., 2018 (“Automatic Dietary Monitoring Using Wearable Accessories”) reviews wearable devices for dietary monitoring. Sen et al., 2018 (“Annapurna: Building a Real-World Smartwatch-Based Automated Food Journal”; access to abstract only) discloses food consumption monitoring using a smart watch with a motion sensor and a camera. Sun et al., 2010 (“A Wearable Electronic System for Objective Dietary Assessment”) discloses food consumption monitoring using a wearable circular device with earphones, microphones, accelerometers, or skin-surface electrodes. Tamura et al., 2016 (“Review of Monitoring Devices for Food Intake”) reviews wearable devices for eating detection and food consumption monitoring. Thomaz et al., 2013 (“Feasibility of Identifying Eating Moments from First-Person Images Leveraging Human Computation”) discloses eating detection through analysis of first-person images. Thomaz et al., 2015 (“A Practical Approach for Recognizing Eating Moments with Wrist-Mounted Inertial Sensing”) discloses eating detection using a smart watch with an accelerometer.

Vu et al., 2017 (“Wearable Food Intake Monitoring Technologies: A Comprehensive Review”) reviews sensing platforms and data analytic approaches to solve the challenges of food-intake monitoring, including ear-based chewing and swallowing detection systems and wearable cameras. Young, 2020 (“FitByte Uses Sensors on Eyeglasses to Automatically Monitor Diet: CMU Researchers Propose a Multimodal System to Track Foods, Liquid Intake”) discloses food consumption monitoring using a device with a motion sensor, an infrared sensor, and a camera which is attached to eyeglasses. Zhang et al., 2016 (“Diet Eyeglasses: Recognising Food Chewing Using EMG and Smart Eyeglasses”; access to abstract only) discloses eating detection using eyeglasses with EMG sensors. Zhang et al., 2018a (“Free-Living Eating Event Spotting Using EMG-Monitoring Eyeglasses”; access to abstract only) discloses eating detection using eyeglasses with EMG sensors. Zhang et al., 2018b (“Monitoring Chewing and Eating in Free-Living Using Smart Eyeglasses”) discloses eating detection using eyeglasses with EMG sensors.

### SUMMARY OF THE INVENTION

This invention can be embodied in methods, systems, and devices for reducing a person's consumption of unhealthy food by providing the person with haptic and/or tactile sensations in response to detection of unhealthy food near the person and/or detection of consumption of unhealthy food by the person. In an example, such a method can include: receiving images from a camera on a device worn by the person, analyzing the images to detect types and/or quantities of nearby food, and providing haptic and/or tactile sensations for the person via at least one haptic and/or tactile component on the wearable device when analysis of the images detects unhealthy types and/or quantities of food near the person. In another example, such a method can include: receiving data from a food consumption sensor on a device worn by a person, analyzing the data to detect types and/or quantities of food consumed by the person, and providing haptic and/or tactile sensations for the person via at least one haptic and/or tactile component on the wearable device when analysis of the data detects consumption of unhealthy types and/or quantities of food by the person.

### BRIEF INTRODUCTION TO THE FIGURES

FIG. 1 shows a method for reducing a person's consumption of unhealthy food by providing haptic and/or tactile sensations when unhealthy food is detected nearby via analysis of images from a camera worn by the person.

FIG. 2 shows a method for reducing a person's consumption of unhealthy food comprising by providing haptic and/or tactile sensations when consumption of unhealthy food by the person is detected by a food consumption sensor worn by the person.

FIGS. 3 and 4 show views, at two different times, of a wearable system and/or device for modifying a person's food consumption comprising a wearable eating detector and a wearable speaker, wherein the wearable speaker plays music or sound tones to modify the person's food consumption based on data from the eating detector.

FIGS. 5 and 6 show views, at two different times, of a wearable system and/or device for modifying a person's food consumption comprising a wearable eating detector and a neurostimulating electrode, wherein the electrode provides neurostimulation to modify the person's food consumption based on data from the eating detector.

FIGS. 7 and 8 show views, at two different times, of a wearable system and/or device for modifying a person's food consumption comprising a wearable eating detector and a word-transmitting speaker, wherein the speaker transmits spoken word stimulus (e.g. encouraging or complimentary words) to modify the person's food consumption based on data from the eating detector.

### DETAILED DESCRIPTION OF THE FIGURES

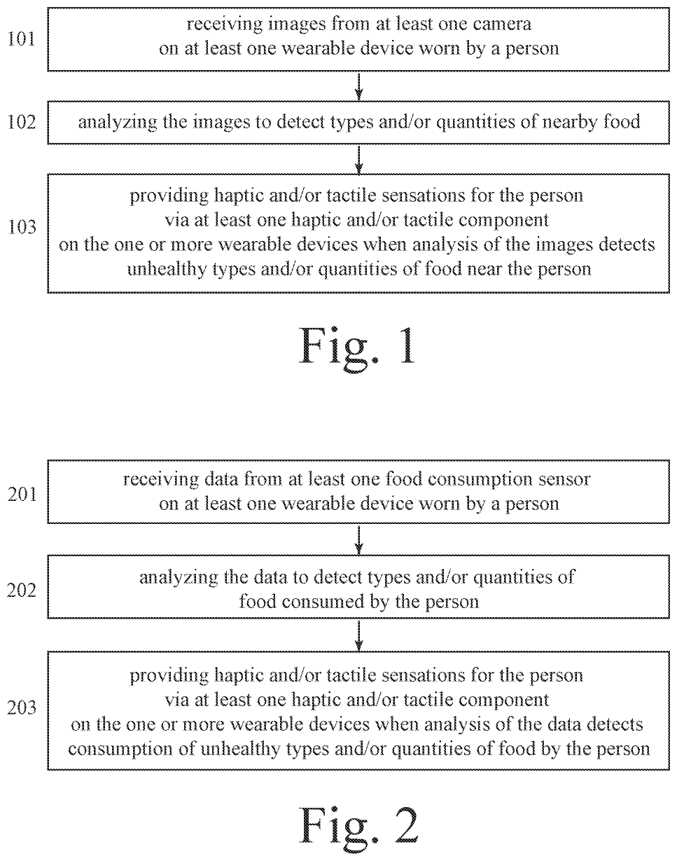

FIG. 1 shows an example of a method for reducing a person's consumption of unhealthy food comprising: receiving images from at least one camera on at least one wearable device worn by a person (step 101), analyzing the images to detect types and/or quantities of nearby food (step 102), and providing haptic and/or tactile sensations for the person via at least one haptic and/or tactile component on the one or more wearable devices when analysis of the images detects unhealthy types and/or quantities of food near the person (step 103). Example variations discussed elsewhere in this disclosure or in priority-linked disclosures can be applied to this example where relevant.

FIG. 2 shows an example of a method for reducing a person's consumption of unhealthy food comprising: receiving data from at least one food consumption sensor on at least one wearable device worn by a person (step 201), analyzing the data to detect types and/or quantities of food consumed by the person (step 202), and providing haptic and/or tactile sensations for the person via at least one haptic and/or tactile component on the one or more wearable devices when analysis of the data detects consumption of unhealthy types and/or quantities of food by the person (step 203). Example variations discussed elsewhere in this disclosure or in priority-linked disclosures can be applied to this example where relevant.

In an example, a method for reducing a person's consumption of unhealthy food can comprise: receiving images from at least one camera on at least one wearable device worn by a person; analyzing the images to detect types and/or quantities of nearby food; and providing haptic and/or tactile sensations for the person via at least one haptic and/or tactile component on the one or more wearable devices when analysis of the images detects unhealthy types and/or quantities of food near the person.

In an example, a method for reducing a person's consumption of unhealthy food can comprise: receiving images from at least one camera (e.g. imaging device) on at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; analyzing the images (e.g. using machine learning and/or artificial intelligence) to detect types and/or quantities of nearby food; and providing haptic (e.g. haptic and/or tactile) sensations for the person via at least one haptic (e.g. haptic and/or tactile) component (e.g. vibrating or shaking component, component with moving protrusions or sections, or electrical energy emitter) on the one or more wearable devices when analysis of the images detects unhealthy types and/or quantities of food near the person.

In an example, a method for reducing a person's consumption of unhealthy food can comprise: receiving data from at least one food consumption sensor on at least one wearable device worn by a person; analyzing the data to detect types and/or quantities of food consumed by the person; and providing haptic and/or tactile sensations for the person via at least one haptic and/or tactile component on the one or more wearable devices when analysis of the data detects consumption of unhealthy types and/or quantities of food by the person.

In an example, a method for reducing a person's consumption of unhealthy food can comprise: receiving data from at least one food consumption sensor (e.g. sound-based eating sensor, light-based/optical eating sensor, muscle/EMG-based eating sensor, brainwave/EEG-based eating sensor, jaw-motion-based eating sensor, body glucose sensor, vibration-based eating sensor, wrist-motion-based eating sensor, hand-gesture-based eating sensor, and/or image-based eating sensor) on at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; analyzing the data (e.g. using machine learning and/or artificial intelligence) to detect types and/or quantities of food consumed by the person; and providing haptic (e.g. haptic and/or tactile) sensations for the person via at least one haptic (e.g. haptic and/or tactile) component (e.g. vibrating or shaking component, component with moving protrusions or sections, or electrical energy emitter) on the one or more wearable devices when analysis of the data detects consumption of unhealthy types and/or quantities of food by the person.

In an example, a system for reducing a person's consumption of unhealthy food can comprise: at least one wearable device worn by a person; at least one camera on the at least one wearable device, wherein the camera captures images of nearby food, and wherein these images are analyzed to detect types and/or quantities of the nearby food; and at least one haptic and/or tactile component on the one or more wearable devices, wherein the haptic component provides haptic sensations for the person when analysis of the images detects unhealthy types and/or quantities of food in the nearby food.

In an example, a system for reducing a person's consumption of unhealthy food can comprise: at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; at least one camera (e.g. imaging device) on the at least one wearable device, wherein the camera captures images of nearby food (e.g. food near the person), and wherein these images are analyzed (e.g. by machine learning and/or artificial intelligence) to detect types and/or quantities of the nearby food; and at least one haptic (e.g. haptic and/or tactile) component (e.g. vibrating or shaking component, component with moving protrusions or sections, or electrical energy emitter) on the one or more wearable devices, wherein the haptic component provides haptic sensations for the person when analysis of the images detects unhealthy types and/or quantities of food in the nearby food.

In an example, a system for reducing a person's consumption of unhealthy food can include one or more moving components on an arm-worn device (e.g. arm band, shirt sleeve, or shirt cuff) which provide the person wearing the device with a haptic (e.g. haptic and/or tactile) sensation through movement of the components in response to nearby and/or consumed unhealthy food, wherein the moving components are located on the side of the device which faces the surface of the person's body. In an example, a method for reducing a person's consumption of unhealthy food can include using a smart watch to provide haptic (e.g. haptic and/or tactile) sensations to a person to reduce the person's consumption of unhealthy food.

In an example, a method for reducing a person's consumption of unhealthy food can comprise: using artificial intelligence and/or machine-learning to analyze data from wearable sensors to measure the type and/or quantity of food consumed by a person during a period of time; using artificial intelligence and/or machine-learning to identify the type and/or quantity food near the person; and using artificial intelligence and/or machine-learning to selected a haptic (e.g. haptic and/or tactile) sensation which is provided to the person via a wearable device, wherein the haptic (e.g. haptic and/or tactile) sensation is based on food consumed by the person and food near the person. In an example, a method for reducing a person's consumption of unhealthy food can comprise using machine learning and/or artificial intelligence to identify the type of food in an image and/or the quantity of food in the image as part of determining whether consumption of that food would be unhealthy for a person.

In an example, a method for reducing a person's consumption of unhealthy food can include using machine learning and/or artificial intelligence to identify relationships between a person's past consumption of specific types and/or quantities of food and subsequent changes in their biometric parameters (e.g. which indicate that those types and/or quantities of food are unhealthy for that person), wherein these relationships are used to selected and provide the most-effective types of haptic and/or tactile sensations for reducing the person's consumption of unhealthy food. In an example, a method for reducing a person's consumption of unhealthy food can comprise: receiving images of food from an imaging device (e.g. a camera) worn by a person; using artificial intelligence and/or machine-learning to identify food in the images which would be unhealthy for the person to eat; and providing a haptic (e.g. haptic and/or tactile) sensation to the person when food in the images would be unhealthy for the person to eat.

In an example, a system for reducing a person's consumption of unhealthy food can record and/or analyze images (e.g. analysis of images from a wearable camera to recognize food). In an example, food near a person can be defined as food within a six-foot radius of the person. In an example, food near a person can be defined as food within the field of vision of a camera worn by the person. In an example, a system for reducing a person's consumption of unhealthy food can include a chewing sensor which measures a person's chewing motions by emitting near-infrared light toward (the surface of) the person's body (e.g. jaw) and receiving this light after it has been reflected from (the surface of) the person's body. In an example, a system for reducing a person's consumption of unhealthy food can include a chewing sensor which is directed toward the person's body and a camera.

In an example, a system for reducing a person's consumption of unhealthy food can include an optical chewing and/or swallowing sensor to detect when the person eats and/or to track how much they eat. In an example, a system for reducing a person's consumption of unhealthy food can include an electromagnetic sensor (e.g. an EMG or EEG sensor) to detect when a person eats and/or to track how much they eat. In an example, a system for reducing a person's consumption of unhealthy food can use machine learning and/or artificial intelligence to track (e.g. monitor, measure, and/or analyze) a person's recent financial transactions (e.g. transactions associated with food consumption). In an example, a system for reducing a person's consumption of unhealthy food can track (e.g. monitor, measure, and/or analyze) body glucose level (e.g. blood glucose level or interstitial glucose level).

In an example, a system for reducing a person's consumption of unhealthy food can use machine learning and/or artificial intelligence to track (e.g. monitor, measure, and/or analyze ambient sounds (e.g. sounds associated with food consumption). In an example, a system for reducing a person's consumption of unhealthy food can use machine learning and/or artificial intelligence to track (e.g. monitor, measure, and/or analyze) day of the week (e.g. day associated with food consumption). In an example, a system for reducing a person's consumption of unhealthy food can use machine learning and/or artificial intelligence to track (e.g. monitor, measure, and/or analyze) hand motions (e.g. hand and/or arm motions associated with food consumption). In an example, a system for reducing a person's consumption of unhealthy food can track (e.g. monitor, measure, and/or analyze) body posture or configuration (e.g. posture associated with food consumption).

In an example, a system for reducing a person's consumption of unhealthy food can track (e.g. monitor, measure, and/or analyze) the geographic location of a person (e.g. location associated with food consumption). In an example, a system for reducing a person's consumption of unhealthy food can track (e.g. monitor, measure, and/or analyze) a person's jaw motions (e.g. jaw motions associated with food consumption). In an example, a system for reducing a person's consumption of unhealthy food can perform spectroscopic analysis (e.g. spectroscopic analysis of nearby food). In an example, a system for reducing a person's consumption of unhealthy food can include a wearable spectroscopy sensor for identification of the (molecular and/or nutritional) composition of nearby food.

In an example, food can be identified as unhealthy because it has a high percentage and/or quantity of fat. In an example, food in an image can be identified as unhealthy for a person to eat based on the person's food allergies. In an example, food of a particular type can be identified as unhealthy because a person is allergic to it. In an example, food of a particular type can be identified as unhealthy because it has a high percentage and/or quantity of fat. In an example, a haptic (e.g. haptic and/or tactile) sensation provided by a system for reducing a person's consumption of unhealthy food can comprise pressure from a contracting band or strip. In an example, a haptic (e.g. haptic and/or tactile) sensation to discourage consumption of unhealthy food can be contraction of a wearable band.

In an example, a haptic (e.g. haptic and/or tactile) component which provides a haptic (e.g. haptic and/or tactile) sensation can be a neurostimulator which transmits a low electrical current to a person's nerves and/or muscles. In an example, a system for reducing a person's consumption of unhealthy food can provide haptic (e.g. haptic and/or tactile) sensations to the person in response to unhealthy food via haptic (e.g. haptic and/or tactile) actuators which are embedded in (e.g. woven or otherwise integrated into) an article of clothing. In an example, a system for reducing a person's consumption of unhealthy food can include haptic (e.g. haptic and/or tactile) actuators which are embedded in (e.g. woven or otherwise integrated into) the cuff and/or sleave of a shirt.

In an example, a system for reducing a person's consumption of unhealthy food can provide haptic (e.g. haptic and/or tactile) sensations to the person in response to unhealthy food via haptic (e.g. haptic and/or tactile) actuators in a wrist band and/or watch band. In an example, a system for reducing a person's consumption of unhealthy food can include haptic (e.g. haptic and/or tactile) actuators in a smart watch. In an example, a system for reducing a person's consumption of unhealthy food can provide haptic (e.g. haptic and/or tactile) sensations to the person in response to unhealthy food via haptic (e.g. haptic and/or tactile) actuators in a bracelet and/or arm band. In an example, a system for reducing a person's consumption of unhealthy food can include haptic (e.g. haptic and/or tactile) actuators in a belt which is worn around the person's waist.

In an example, a method for reducing a person's consumption of unhealthy food can comprise providing haptic (e.g. haptic and/or tactile) sensations in response to unhealthy food via haptic (e.g. haptic and/or tactile) actuators in an adhesive patch. In an example, a method for reducing a person's consumption of unhealthy food can comprise providing haptic (e.g. haptic and/or tactile) sensations in response to unhealthy food via haptic (e.g. haptic and/or tactile) actuators in a finger ring. In an example, a system for reducing a person's consumption of unhealthy food can include one or more moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are rotated in a plane which is substantially parallel to the surface of the person's body.

In an example, a system for reducing a person's consumption of unhealthy food can include one or more moving components (e.g. protrusions, bumps, rollers, pins, or teeth) on a wearable device which provide the person wearing the device with a haptic (e.g. haptic and/or tactile) sensation through movement in response to nearby and/or consumed unhealthy food, wherein the moving components are located on the side of the device which faces the surface of the person's body. In an example, a system for reducing a person's consumption of unhealthy food can comprise one or more moving protrusions on the side of a watch band, wrist band, or bracelet which faces toward the surface of the person's body, wherein the protrusions move in a direction which is substantially-parallel to the surface of the person's body.

In an example, a system for reducing a person's consumption of unhealthy food can comprise one or more moving protrusions on the side of a smart watch which faces toward the surface of the person's body, wherein the protrusions move in a direction which is substantially-parallel to the surface of the person's body. In an example, a haptic (e.g. haptic and/or tactile) sensation can be provided by movement of a plurality of protrusions (e.g. bumps, dots, teeth, pins, or legs) which extend out from a wearable device toward a person's body.

In an example, a system for reducing a person's consumption of unhealthy food can comprise: at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; at least one food consumption sensor (e.g. sound-based eating sensor, light-based/optical eating sensor, muscle/EMG-based eating sensor, brainwave/EEG-based eating sensor, jaw-motion-based eating sensor, body glucose sensor, vibration-based eating sensor, wrist-motion-based eating sensor, hand-gesture-based eating sensor, and/or image-based eating sensor) on the at least one wearable device, wherein data from the food consumption sensor is analyzed (e.g. by machine learning and/artificial intelligence) to detect the types and/or quantities of food (or specific nutrients in food) consumed by the person (e.g. consumed during a period of time); and at least one haptic (e.g. haptic and/or tactile) component (e.g. vibrating or shaking component, component with moving protrusions or sections, or electrical energy emitter) on the one or more wearable devices, wherein the haptic component provides haptic sensations for the person when analysis of data from the one or more food consumption sensors detects consumption of unhealthy types and/or quantities of food by the person.

In an example, haptic and/or tactile sensations in response to unhealthy food (detected nearby and/or consumed by a person) can be provided to the person by an array of moving components on a wearable device which is worn by the person. In an example, a system for reducing a person's consumption of unhealthy food can include an orthogonal array (e.g. matrix or grid) of moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are located on the side of the device which faces the surface of the person's body.

In an example, a haptic (e.g. haptic and/or tactile) sensation can be provided by the movement of a selected subset of an array (e.g. array, matrix, or grid) of individually-movable protrusions (e.g. bumps, dots, teeth, pins, or legs) which extend out from a wearable device toward a person's body, wherein the subset is selected by based on the type and/or quantity of unhealthy food identified in images, and wherein the movement is lateral (e.g. parallel to) the surface of the person's body.

In an example, a system for reducing a person's consumption of unhealthy food can include moving components (e.g. protrusions, bumps, rollers, pins, or teeth) on a wearable device which provide the person wearing the device with a haptic (e.g. haptic and/or tactile) sensation through movement of the components in response to nearby unhealthy food detected in an image and/or detection of consumption of unhealthy food by the person, wherein the moving components are located on the side of the device which faces the surface of the person's body, and wherein a subset of the moving components can be selectively moved, and wherein selection of the subset of moving components is based on the type and/or quantity of unhealthy food detected.

In an example, a method for reducing a person's consumption of unhealthy food can comprise: receiving images from at least one camera (e.g. imaging device) on at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; using machine learning and/or artificial intelligence to analyze the images to detect types and/or quantities of nearby food; receiving data from at least one food consumption sensor (e.g. light-based eating sensor, sound-based eating sensor, EMG-based eating sensor, EEG-based eating sensor, jaw-motion-based eating sensor, body glucose sensor, vibration-based eating sensor, wrist-motion-based eating sensor, hand-gesture-based eating sensor, and/or image-based eating sensor) on the at least one wearable device; using machine learning and/or artificial intelligence to analyze the data from the food consumption sensor to detect the types and/or quantities of food (or specific nutrients in food) consumed by the person (e.g. consumed during a period of time); and providing haptic (e.g. haptic and/or tactile) sensations for the person via at least one haptic (e.g. haptic and/or tactile) component (e.g. vibrating or shaking component, component with moving protrusions or sections, or electrical energy emitter) on the one or more wearable devices when analysis of the images detects unhealthy types and/or quantities of food near the person; and/or when analysis of data from the one or more food consumption sensors detects consumption of unhealthy types and/or quantities of food by the person.

In an example, a haptic (e.g. haptic and/or tactile) sensation provided by a system for a person can comprise one or more vibrating components which are worn by the person. In an example, a haptic (e.g. haptic and/or tactile) sensation to discourage consumption of unhealthy food can be a specific sequence of vibrations. In an example, a haptic (e.g. haptic and/or tactile) sensation to discourage consumption of unhealthy food can be a vibration, wherein the location and/or size of the vibration is based in part on the type of unhealthy food detected. In an example, a haptic (e.g. haptic and/or tactile) sensation to discourage consumption of unhealthy food can be a vibration, wherein the duration and/or length of the vibration is based in part on the quantity of unhealthy food detected.

In an example, a haptic (e.g. haptic and/or tactile) component which provides a haptic (e.g. haptic and/or tactile) sensation can be a piezoelectric component which vibrates, contracts, and/or otherwise moves. In an example, a system for reducing a person's consumption of unhealthy food can comprise one or more vibrating protrusions on the side of a watch band, wrist band, or bracelet which faces toward the surface of the person's body. In an example, a haptic (e.g. haptic and/or tactile) component which provides a haptic (e.g. haptic and/or tactile) sensation can be a vibrating component (e.g. a vibrating protrusion, bump, dot, panel, or band). In an example, a haptic (e.g. haptic and/or tactile) sensation to discourage consumption of unhealthy food can be a vibration, wherein the frequency and/or rate of the vibration is based in part on the type of unhealthy food detected.

In an example, a vibrating or otherwise moving component for providing a haptic and/or tactile sensation can be part of, or attached to, an earring. In an example, a vibrating or otherwise moving component for providing a haptic and/or tactile sensation can be part of, or attached to, an adhesive patch. In an example, a vibrating or otherwise moving component for providing a haptic and/or tactile sensation can be part of, or attached to, a smart watch. In an example, a vibrating or otherwise moving component for providing a haptic and/or tactile sensation can be part of, or attached to, a necklace or pendant. In an example, a vibrating or otherwise moving component for providing a haptic and/or tactile sensation can be part of, or attached to, a bracelet. In an example, a method for reducing a person's consumption of unhealthy food can include using a vibrating smart watch to provide haptic (e.g. haptic and/or tactile) sensations to a person to reduce the person's consumption of unhealthy food.

In an example, a haptic (e.g. haptic and/or tactile) sensation can comprise a selected sequential pattern of component movements or vibrations, wherein the pattern is based on the type and/or quantity of unhealthy food near a person (e.g. detected via analysis of images by machine learning and/or artificial intelligence) and/or unhealthy food consumed by the person (e.g. detected via a chewing sensor and/or analysis of images by machine learning and/or artificial intelligence). In an example, a haptic (e.g. haptic and/or tactile) sensation to discourage consumption of unhealthy food can be a vibration, wherein the magnitude and/or intensity of the vibration is based in part on the type of unhealthy food detected.

In an example, a system for reducing a person's consumption of unhealthy food can include smart eyewear or an eyewear attachment, wherein the magnitude, frequency, and/or pattern of vibration is based on the type and/or quantity of unhealthy food that is detected as being nearby and/or having been consumed by the person wearing the eyewear. In an example, a system for reducing a person's consumption of unhealthy food can include a smart ring which vibrates, wherein the magnitude, frequency, and/or pattern of the vibration is based on the type and/or quantity of unhealthy food that is detected as being nearby and/or having been consumed by the person wearing the ring. In an example, a haptic (e.g. haptic and/or tactile) sensation provided by a system for a person can an arcuate array of vibrating components which is worn by a person.

In an example, a haptic (e.g. haptic and/or tactile) sensation can comprise a selected a sequential pattern of (an arcuate array of) component movements or vibrations, wherein the pattern is based on the type and/or quantity of unhealthy food near a person (e.g. detected via analysis of images) and/or unhealthy food consumed by the person (e.g. detected via a chewing sensor and/or analysis of images). In an example, a haptic (e.g. haptic and/or tactile) sensation can be provided by the vibration or other movement of a selected subset of an arcuate array of individually-movable protrusions (e.g. bumps, dots, teeth, pins, or legs) which extend out from a wearable device toward a person's body, wherein the subset is selected by machine learning and/or artificial intelligence based on the type and/or quantity of food nearby and/or consumed by the person.

In an example, a haptic (e.g. haptic and/or tactile) component which provides a haptic (e.g. haptic and/or tactile) sensation can be an orthogonal array (e.g. grid or matrix) of vibrating components (e.g. a vibrating protrusions, bumps, dots, pins, prongs, or teeth). In an example, a device worn on a person's wrist and/or arm to provide haptic (e.g. haptic and/or tactile) sensations in response to detection of unhealthy food near the person and/or unhealthy food consumed by the person, wherein this device includes an arcuate array of haptic actuators (e.g. vibrating and/or moving components) which span (a portion of) the circumference of the person's wrist and/or arm, wherein these haptic actuators are actuated in a selected radial, sequential pattern around (a portion of) the circumference of the person's wrist and/or arm, and wherein the selected radial, sequential pattern is based on the type and/or quantity of unhealthy food detected.

In an example, a device worn on a person's wrist and/or arm to provide haptic (e.g. haptic and/or tactile) sensations in response to detection of unhealthy food near the person and/or unhealthy food consumed by the person, wherein this device includes an arcuate array of haptic actuators (e.g. vibrating and/or moving components) which span a portion of the circumference of the person's wrist and/or arm, wherein these haptic actuators are actuated in a radial sequential pattern around a portion of the circumference of the person's wrist and/or arm.

In an example, a system for reducing a person's consumption of unhealthy food can include one or more moving components on a wrist-worn device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) which provide the person wearing the device with a haptic (e.g. haptic and/or tactile) sensation through movement of the components in response to nearby and/or consumed unhealthy food, wherein the moving components are located on the side of the device which faces the surface of the person's body.

In an example, a system for reducing a person's consumption of unhealthy food can comprise a finger ring with a camera to record food images. In an example, a system for reducing a person's consumption of unhealthy food can include a watch band which is used to provide haptic (e.g. haptic and/or tactile) sensations to a person to reduce the person's consumption of unhealthy food. In an example, a wearable camera for detection of nearby unhealthy food can be part of, or attached to, an earring. In an example, a wearable camera for detection of nearby unhealthy food can be part of, or attached to, an adhesive patch. In an example, a wearable camera for detection of nearby unhealthy food can be part of, or attached to, a smart watch. In an example, a wearable camera for detection of nearby unhealthy food can be part of, or attached to, a necklace or pendant.

In an example, a wearable camera for detection of nearby unhealthy food can be part of, or attached to, a bracelet. In an example, a system for reducing a person's consumption of unhealthy food can comprise augmented reality (AR) eyewear with bilateral cameras to record food images. In an example, a wearable camera can be integrated into smart eyewear (e.g. smart eyeglasses). In an example, a system for reducing a person's consumption of unhealthy food can include haptic (e.g. haptic and/or tactile) actuators on eyewear (e.g. eyeglasses). In an example, a system for reducing a person's consumption of unhealthy food can include a chewing sensor which is attached to the sidepiece (e.g. temple) of eyeglasses worn by the person.

In an example, a system for reducing a person's consumption of unhealthy food can include a chewing sensor which is directed toward the person's body and a camera which is directed away from the person's body, wherein the chewing sensor and the camera are attached to eyeglasses worn by the person. In an example, a wearable camera for detection of nearby unhealthy food can be part of, or attached to, AR eyewear. In an example, an imaging device (e.g. camera) can be a modular component which is removably-attached to eyewear (e.g. eyeglasses) worn by the person. In an example, an imaging device (e.g. camera) can be part of, or attached to, the front piece of eyewear (e.g. eyeglasses) worn by the person.

In an example, an imaging device (e.g. camera) can be part of, or attached to, augmented reality eyewear (e.g. AR eyeglasses) worn by the person. In an example, a system for reducing a person's consumption of unhealthy food can track hand motions and trigger a haptic (e.g. haptic and/or tactile) sensation if a person's hand motions relative to unhealthy food indicate that the person is eating (or intending to eat) the unhealthy food. In an example, a system for reducing a person's consumption of unhealthy food can track a person's hand in order to detect one or more hand motions selected from the group consisting of: bringing unhealthy food up to their mouth, grasping unhealthy food, reaching toward unhealthy food, and using a utensil to engage (e.g. cut or lift) unhealthy food—wherein the system responds to detection of one or more of these hand motions by providing haptic (e.g. haptic and/or tactile) feedback to the person.

In an example, food near a person can be defined as food within 3 feet of the person's hands and within the field of vision of a camera worn by the person. In an example, the focal vector of an imaging device (e.g. camera) which is worn by a person can track the configuration of a person's hand (or hands) in order to identify nearby food by eating-related hand configurations (e.g. gestures). In an example, a system for reducing a person's consumption of unhealthy food can comprise a first device worn on a person's dominant wrist and/or arm and a second device worn on the person's non-dominant wrist and/or arm. In an example, a system for reducing a person's consumption of unhealthy food can comprise a wearable camera to track arm and/or hand motions and/or gestures to identify when a person is eating and/or how much they are eating.

In an example, the location of haptic (e.g. haptic and/or tactile) sensation provided by a system for reducing a person's consumption of unhealthy food can depend on the type of unhealthy food detected (e.g. type of unhealthy food detected nearby, detected in proximity to a person's hand, or detected to have been consumed by the person thus far). In an example, the pattern of haptic (e.g. haptic and/or tactile) sensation provided by a system for reducing a person's consumption of unhealthy food can depend on the quantity of unhealthy food detected (e.g. quantity of unhealthy food detected nearby, detected in proximity to a person's hand, or detected to have been consumed by the person thus far). In an example, the magnitude of a haptic (e.g. haptic and/or tactile) sensation provided to a person can increase with the proximity of the person's hand to unhealthy food.

In an example, the magnitude of haptic (e.g. haptic and/or tactile) sensation provided by a system for reducing a person's consumption of unhealthy food can depend on the quantity of unhealthy food detected (e.g. quantity of unhealthy food detected nearby, detected in proximity to a person's hand, or detected to have been consumed by the person thus far). In an example, the rate and/or frequency of haptic (e.g. haptic and/or tactile) sensation provided by a system for reducing a person's consumption of unhealthy food can depend on the quantity of unhealthy food detected (e.g. quantity of unhealthy food detected nearby, detected in proximity to a person's hand, or detected to have been consumed by the person thus far).

In an example, a method for reducing a person's consumption of unhealthy food can comprise: using artificial intelligence and/or machine-learning to analyze data from wearable sensors to measure unhealthy food consumed by a person; using artificial intelligence and/or machine-learning to identify unhealthy food near the person; and using artificial intelligence and/or machine-learning to selected a haptic (e.g. haptic and/or tactile) sensation which is provided to the person via a wearable device, wherein the haptic (e.g. haptic and/or tactile) sensation is selected based on joint analysis of unhealthy food consumed by the person and unhealthy food near the person. In an example, a method for reducing a person's consumption of unhealthy food can comprise: receiving images of food from an imaging device (e.g. a camera) worn by a person; using artificial intelligence and/or machine-learning to identify unhealthy food in the images; and using artificial intelligence and/or machine-learning to selected a haptic (e.g. haptic and/or tactile) sensation which is provided to the person based on the type and/or quantity of unhealthy food identified in the images.

In an example, a method for reducing a person's consumption of unhealthy food can comprise: using artificial intelligence and/or machine-learning to analyze images to identify unhealthy food near a person; and using artificial intelligence and/or machine-learning to selected a haptic (e.g. haptic and/or tactile) sensation which is provided to the person via a wearable device, wherein the type of haptic (e.g. haptic and/or tactile) sensation is based on the type of unhealthy food identified in the images. In an example, the magnitude of haptic (e.g. haptic and/or tactile) sensation provided by a system for reducing a person's consumption of unhealthy food can depend on the proximity of unhealthy food to a person's mouth.

In an example, a method for reducing a person's consumption of unhealthy food can comprise: using artificial intelligence and/or machine-learning to analyze images to identify unhealthy food near a person; and using artificial intelligence and/or machine-learning to selected a haptic (e.g. haptic and/or tactile) sensation which is provided to the person via a wearable device, wherein the magnitude of haptic (e.g. haptic and/or tactile) sensation is based on the quantity of unhealthy food identified in the images. In an example, the frequency or rate of haptic (e.g. haptic and/or tactile) sensations provided to a person can be increased with the potential harmfulness of nearby food (e.g. analogous to the increased frequency of sounds from a Geiger counter based on the strength of a radioactive source).

In an example, the rate and/or frequency of haptic (e.g. haptic and/or tactile) sensation provided by a system for reducing a person's consumption of unhealthy food can depend on the proximity of unhealthy food to a person's mouth. In an example, the magnitude of a haptic (e.g. haptic and/or tactile) sensation provided to a person can be increased with the quantity of unhealthy food consumed by the person (e.g. analogous to the increased frequency of sounds from a Geiger counter based on the strength of a radioactive source). In an example, a person wearing a device to provide haptic (e.g. haptic and/or tactile) sensations can deactivate the device by one or more mechanisms selected from the following group: a voice command, contracting one or more muscles in a selected manner, making a selected hand gesture, pressing a button on the device, tapping the device, and touching or swiping a screen on the device.

In an example, a person wearing a system can increase or decrease the magnitude of haptic (e.g. haptic and/or tactile) sensation provided by the system (e.g. for a selected period of time). In an example, a person wearing a system can increase or decrease the sensitivity of the system to detection of unhealthy food (e.g. for a selected period of time) by pressing a button or touching a screen. In an example, a system for reducing a person's consumption of unhealthy food can have an adjustable sensitivity level, wherein the magnitude and/or type of haptic (e.g. haptic and/or tactile) sensation provided to the person can be changed (e.g. increased or decreased) by the person wearing the device. For example, if the person is at a holiday or other social event in which they wish to temporarily reduce the amount of feedback related to unhealthy types or quantities of food, then they can reduce the magnitude of haptic (e.g. haptic and/or tactile) sensation provided by the system.

In an example, machine learning and/or artificial reality can be used to analyze a person's environment and/or social context in order to automatically adjust the magnitude or form of haptic (haptic and/or tactile) sensations which are provided to the person in response to detection of nearby unhealthy food and/or consumption of unhealthy food by the person. In an example, a method for reducing a person's consumption of unhealthy food can include adjusting the type and/or magnitude of haptic (e.g. haptic and/or tactile) sensation provided to a person based on analysis of the effectiveness of past types and/or magnitudes of haptic (e.g. haptic and/or tactile) sensations provided to that person.

In an example, a system for reducing a person's consumption of unhealthy food can further comprise an eating sensor (e.g. a chewing and/or swallowing sensor) which detects eating, wherein the system triggers a haptic (e.g. haptic and/or tactile) sensation and/or wherein analysis of data from the eating sensor indicates that the person has eaten an unhealthy amount of food during a selected time period (e.g. during a meal). In an example, a method for reducing a person's consumption of unhealthy food can comprise using machine learning and/or artificial intelligence to evaluate whether consumption of food by a person would be unhealthy, wherein this evaluation is based in part on the types and/or amounts of food consumed recently (e.g. during a recent period of time) by the person.

In an example, a system for reducing a person's consumption of unhealthy food can include using machine learning and/or artificial intelligence to determine when a person is consuming unhealthy food, wherein one or more of the following factors are used by machine learning and/or artificial intelligence to make this determination: ambient sounds (e.g. sounds associated with food consumption); analysis of images (e.g. analysis of images from a wearable camera to recognize food); body posture or configuration (e.g. posture associated with food consumption); body glucose level (e.g. blood glucose level or interstitial glucose level); body sounds (e.g. chewing and/or swallowing sounds associated with food consumption); cellphone activity (e.g. cellphone activity associated with food consumption); day of the week (e.g. day associated with food consumption); geographic location of the person (e.g. location associated with food consumption); hand gestures (e.g. hand gestures associated with food consumption); hand motions (e.g. hand and/or arm motions associated with food consumption); jaw motions (e.g. jaw motions associated with food consumption); recent financial transactions (e.g. transactions associated with food consumption); room location (e.g. room in a building associated with food consumption); spectroscopic analysis (e.g. spectroscopic analysis of nearby food); and time of day (e.g. time of day associated with food consumption).

In an example, a system for reducing a person's consumption of unhealthy food can use machine learning and/or artificial intelligence to track (e.g. monitor, measure, and/or analyze) a person's room location (e.g. room in a building associated with food consumption). In an example, a system for reducing a person's consumption of unhealthy food can further comprise an eating sensor (e.g. a hand or arm motion sensor) which detects eating, wherein the system triggers a wearable imaging device (e.g. camera) to start recording images when analysis of data from the eating detector indicates that the person is eating. In an example, a system for reducing a person's consumption of unhealthy food can include a wearable camera to record images for detection of unhealthy food, wherein machine learning and/or artificial intelligence is used to determine when the camera is triggered (e.g. triggered, turned on, activated) to start recording images.

In an example, a wearable imaging device (e.g. camera) can be triggered to start recording images when analysis of data from an implanted sensor indicates that a person is eating. In an example, a wearable imaging device (e.g. camera) can be triggered to start recording images when analysis of data from a wearable sensor indicates that a person is eating or drinking. In an example, a wearable imaging device (e.g. camera) can be triggered to start recording images when analysis of data from a sensor in a person's mouth indicates that a person is eating. In an example, a wearable imaging device (e.g. camera) can be triggered to start recording images when an implanted sensor detects that a person is eating. In an example, a wearable imaging device (e.g. camera) can be triggered to start recording images when a wearable sensor detects that a person is eating or drinking. In an example, a wearable imaging device (e.g. camera) can be triggered to start recording images when a wearable optical sensor detects that a person is eating.

In an example, a system for reducing a person's consumption of unhealthy food can include a wearable camera to record images for detection of unhealthy food, wherein machine learning and/or artificial intelligence is used to determine when the camera is triggered (e.g. triggered, turned on, activated) to start recording images, wherein one or more of the following factors are used by machine learning and/or artificial intelligence to determine when to trigger the camera: ambient sounds (e.g. sounds associated with food consumption); body posture or configuration (e.g. posture associated with food consumption); body glucose level (e.g. blood glucose level or interstitial glucose level); body sounds (e.g. chewing and/or swallowing sounds associated with food consumption); cellphone activity (e.g. cellphone activity associated with food consumption); geographic location of the person (e.g. location associated with food consumption); hand motions (e.g. hand and/or arm motions associated with food consumption); jaw motions (e.g. jaw motions associated with food consumption); recent financial transactions (e.g. transactions associated with food consumption); room location (e.g. room in a building associated with food consumption); time of day (e.g. time of day associated with food consumption); and day of the week (e.g. day associated with food consumption).

In an example, a wearable system can comprise a data processing hub which is worn on one location on a person's body and a plurality of sensors on other locations on the person's body, wherein there is (wireless) electronic communication between the data processing hub and the sensors, and wherein at least one of the sensors detects when the person consumes food. In an example, a system for reducing a person's consumption of unhealthy food can use machine learning and/or artificial intelligence to analyze time of day (e.g. time of day associated with food consumption). In an example, machine learning and/or artificial intelligence can be used to identify which types of haptic (e.g. haptic and/or tactile) sensations are most effective in reducing a person's consumption of unhealthy food.

In an example, machine learning and/or artificial intelligence can analyze the relationships between haptic (e.g. haptic and/or tactile) sensations provided to a person in the past and unhealthy food consumed by that person in the past in order to identify which types of haptic (e.g. haptic and/or tactile) sensations are most effective in reducing that person's consumption of unhealthy food. In an example, a method for reducing a person's consumption of unhealthy food can include using a contracting watch band to provide haptic (e.g. haptic and/or tactile) sensations to a person to reduce the person's consumption of unhealthy food. In an example, a method for reducing a person's consumption of unhealthy food can include using a watch band to provide haptic (e.g. haptic and/or tactile) sensations to a person to reduce the person's consumption of unhealthy food.

In an example, a method for reducing a person's consumption of unhealthy food can include using machine learning and/or artificial intelligence to provide a person with a first haptic and/or tactile sensation if it is detected that unhealthy food is near the person and to provide the person with a second haptic and/or tactile sensation if it is detected that the person is consuming unhealthy food, wherein the second sensation has a larger magnitude, a longer duration, and/or a larger scope than the first sensation. In an example, a method for reducing a person's consumption of unhealthy food can include: using machine learning and/or artificial intelligence to identify the most-effective types and/or magnitudes of haptic (e.g. haptic and/or tactile) sensations for reducing a person's consumption of unhealthy food in the past; and using these results to select the most effective type and/or magnitude of haptic (e.g. haptic and/or tactile) sensation to reduce consumption of unhealthy food by that person now.

In an example, a method for reducing a person's consumption of unhealthy food can comprise using machine learning and/or artificial intelligence to evaluate whether consumption of food by a person would be unhealthy, wherein this evaluation is based in part on the person's food allergies. In an example, a method for reducing a person's consumption of unhealthy food can comprise: receiving images of food from an imaging device (e.g. a camera) worn by a person; using artificial intelligence and/or machine-learning to identify food in the images which would be unhealthy for the person to eat; and providing a haptic (e.g. haptic and/or tactile) sensation to the person when food in the images would be unhealthy for the person to eat.

In an example, a system for reducing a person's consumption of unhealthy food can estimate the amount of food consumed by a person based, at least in part, on changes in the volume of nearby food in images recorded by a wearable camera. In an example, food near a person can be defined as food within six feet of the person and within the field of vision of a camera worn by the person. In an example, a system for reducing a person's consumption of unhealthy food can estimate the amount of food consumed by a person based on a combination of: changes in the volume of nearby food in images recorded by a wearable camera; hand-to-mouth motions recorded by the camera; and chewing motions measured by a chewing sensor. In an example, a system for reducing a person's consumption of unhealthy food can include a chewing sensor which measures a person's chewing motions by emitting light toward (the surface of) the person's body (e.g. jaw) and receiving this light after it has been reflected from (the surface of) the person's body.

In an example, a system for reducing a person's consumption of unhealthy food can include an electromagnetic sensor to detect when the person eats and/or to track how much they eat. In an example, a system for reducing a person's consumption of unhealthy food can include an optical chewing and/or swallowing sensor (e.g. with a light emitter and light detector) to detect when the person eats and/or to track how much they eat. In an example, a system for reducing a person's consumption of unhealthy food can provide a haptic (e.g. haptic and/or tactile) sensation to a person within three minutes of detection of unhealthy food consumption by the person based on analysis of data from a chewing sensor (and analysis of images from a camera) worn by the person. In an example, a system for reducing a person's consumption of unhealthy food can track (e.g. monitor, measure, and/or analyze) a person's recent financial transactions (e.g. transactions associated with food consumption).

In an example, a system for reducing a person's consumption of unhealthy food can use machine learning and/or artificial intelligence to track (e.g. monitor, measure, and/or analyze) cellphone activity (e.g. cellphone activity associated with food consumption). In an example, a system for reducing a person's consumption of unhealthy food can use machine learning and/or artificial intelligence to track (e.g. monitor, measure, and/or analyze) body posture or configuration (e.g. posture associated with food consumption). In an example, a system for reducing a person's consumption of unhealthy food can use machine learning and/or artificial intelligence to track (e.g. monitor, measure, and/or analyze) the geographic location of a person (e.g. location associated with food consumption).

In an example, a system for reducing a person's consumption of unhealthy food can use machine learning and/or artificial intelligence to track (e.g. monitor, measure, and/or analyze) a person's jaw motions (e.g. jaw motions associated with food consumption). In an example, a system for reducing a person's consumption of unhealthy food can track (e.g. monitor, measure, and/or analyze) body sounds (e.g. chewing and/or swallowing sounds associated with food consumption). In an example, a system for reducing a person's consumption of unhealthy food can track (e.g. monitor, measure, and/or analyze) hand gestures (e.g. hand gestures associated with food consumption). In an example, a system for reducing a person's consumption of unhealthy food can use machine learning and/or artificial intelligence to analyze body glucose level (e.g. blood glucose level or interstitial glucose level).

In an example, a system for reducing a person's consumption of unhealthy food can further comprise a handheld spectroscopy sensor for identification of the (molecular and/or nutritional) composition of nearby food. In an example, food can be identified as unhealthy because it has a high percentage and/or quantity of impurities. In an example, food can be identified as unhealthy because it has a high percentage and/or quantity of sugar. In an example, food in an image can be identified as unhealthy for a person to eat based on a chronic health condition which the person has. In an example, food of a particular type can be identified as unhealthy because it has a high percentage and/or quantity of carbohydrates.

In an example, food of a particular type can be identified as unhealthy because a portion of the food is excessively large. In an example, a haptic (e.g. haptic and/or tactile) sensation provided by a system for reducing a person's consumption of unhealthy food can comprise pressure from a contracting wrist band, bracelet, and/or watch band. In an example, a haptic (e.g. haptic and/or tactile) component which provides a haptic (e.g. haptic and/or tactile) sensation can comprise a solenoid, electromagnetic piston, or other electrical actuator. In an example, a haptic (e.g. haptic and/or tactile) sensation can be accompanied by a sound. In an example, a system for reducing a person's consumption of unhealthy food can provide haptic (e.g. haptic and/or tactile) sensations to the person in response to unhealthy food via haptic (e.g. haptic and/or tactile) actuators which are embedded in (e.g. woven or otherwise integrated into) the cuff and/or sleave of a shirt.

In an example, a system for reducing a person's consumption of unhealthy food can include haptic (e.g. haptic and/or tactile) actuators on an adhesive patch. In an example, a system for reducing a person's consumption of unhealthy food can include haptic (e.g. haptic and/or tactile) actuators in a wrist band and/or watch band. In an example, a system for reducing a person's consumption of unhealthy food can provide haptic (e.g. haptic and/or tactile) sensations to the person in response to unhealthy food via haptic (e.g. haptic and/or tactile) actuators in a finger ring. In an example, a system for reducing a person's consumption of unhealthy food can include haptic (e.g. haptic and/or tactile) actuators in a bracelet and/or arm band.

In an example, a method for reducing a person's consumption of unhealthy food can comprise providing haptic (e.g. haptic and/or tactile) sensations in response to unhealthy food via haptic (e.g. haptic and/or tactile) actuators which are embedded in (e.g. woven or otherwise integrated into) an article of clothing. In an example, a method for reducing a person's consumption of unhealthy food can comprise providing haptic (e.g. haptic and/or tactile) sensations in response to unhealthy food via haptic (e.g. haptic and/or tactile) actuators in a wrist band and/or watch band. In an example, a method for reducing a person's consumption of unhealthy food can comprise providing haptic (e.g. haptic and/or tactile) sensations in response to unhealthy food via haptic (e.g. haptic and/or tactile) actuators in a bracelet and/or arm band.

In an example, a system for reducing a person's consumption of unhealthy food can include one or more moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are moved (e.g. slid) in a direction which is substantially parallel to the surface of the person's body. In an example, a system for reducing a person's consumption of unhealthy food can include one or more moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are located on the side of the device which faces the surface of the person's body.

In an example, a system for reducing a person's consumption of unhealthy food can comprise one or more moving protrusions on the side of a watch band, wrist band, or bracelet which faces toward the surface of the person's body, wherein the protrusions move in a direction which is substantially-perpendicular to the surface of the person's body. In an example, a system for reducing a person's consumption of unhealthy food can comprise one or more moving protrusions on the side of a smart watch which faces toward the surface of the person's body, wherein the protrusions move in a direction which is substantially-perpendicular to the surface of the person's body.

In an example, a system for reducing a person's consumption of unhealthy food can comprise: at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; at least one camera (e.g. imaging device) on the at least one wearable device, wherein the camera captures images of nearby food (e.g. food near the person), and wherein these images are analyzed to detect types and/or quantities of nearby food; at least one food consumption sensor (e.g. light-based eating sensor, sound-based eating sensor, EMG-based eating sensor, EEG-based eating sensor, jaw-motion-based eating sensor, body glucose sensor, vibration-based eating sensor, wrist-motion-based eating sensor, hand-gesture-based eating sensor, and/or image-based eating sensor) on the at least one wearable device, wherein data from the food consumption sensor is analyzed to detect the types and/or quantities of food (or specific nutrients in food) consumed by the person (e.g. consumed during a period of time); and at least one haptic (e.g. haptic and/or tactile) component (e.g. vibrating or shaking component, component with moving protrusions or sections, or electrical energy emitter) on the one or more wearable devices, wherein the haptic component provides haptic sensations for the person when: analysis of the images detects unhealthy types and/or quantities of food near the person; and/or when analysis of data from the one or more food consumption sensors detects consumption of unhealthy types and/or quantities of food by the person.

In an example, haptic and/or tactile sensations can be provided to a person by an array of piezoelectric actuators on a wearable device which is worn by the person. In an example, a system for reducing a person's consumption of unhealthy food can include an array of moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are located on the side of the device which faces the surface of the person's body.

In an example, a system for reducing a person's consumption of unhealthy food can include an orthogonal array (e.g. matrix or grid) of moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are located on the side of the device which faces the surface of the person's body, and wherein a subset of the moving components can be selectively and individually moved. In an example, a haptic (e.g. haptic and/or tactile) sensation can be provided by the movement of a selected subset of an array (e.g. array, matrix, or grid) of individually-movable protrusions (e.g. bumps, dots, teeth, pins, or legs) which extend out from a wearable device toward a person's body, wherein the subset is selected by based on the type and/or quantity of unhealthy food identified in images, and wherein the movement is orthogonal (e.g. perpendicular to) the surface of the person's body.

In an example, a system for reducing a person's consumption of unhealthy food can include moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are located on the side of the device which faces the surface of the person's body, and wherein a subset of the moving components can be selectively and individually moved, and wherein selection of the subset of moving components is based on the type and/or quantity of nearby and/or consumed unhealthy food.

In an example, a method for reducing a person's consumption of unhealthy food can comprise: receiving data from at least one food consumption sensor (e.g. light-based eating sensor, sound-based eating sensor, EMG-based eating sensor, EEG-based eating sensor, jaw-motion-based eating sensor, body glucose sensor, vibration-based eating sensor, wrist-motion-based eating sensor, hand-gesture-based eating sensor, and/or image-based eating sensor) on the at least one wearable device; and using machine learning and/or artificial intelligence to analyze the data from the food consumption sensor to detect the types and/or quantities of food (or specific nutrients in food) consumed by the person (e.g. consumed during a period of time); and providing haptic (e.g. haptic and/or tactile) sensations for the person via at least one haptic (e.g. haptic and/or tactile) component (e.g. vibrating or shaking component, component with moving protrusions or sections, or electrical energy emitter) on the one or more wearable devices when analysis of data from the one or more food consumption sensors detects consumption of unhealthy types and/or quantities of food by the person.

In an example, a haptic (e.g. haptic and/or tactile) sensation provided by a system for reducing a person's consumption of unhealthy food can comprise vibrations. In an example, a haptic (e.g. haptic and/or tactile) sensation to discourage consumption of unhealthy food can be a vibration. In an example, a haptic (e.g. haptic and/or tactile) sensation to discourage consumption of unhealthy food can be a vibration, wherein the location and/or size of the vibration is based in part on the quantity of unhealthy food detected. In an example, a system for reducing a person's consumption of unhealthy food can include a vibrating component on the side of a watch band, wrist band, or bracelet which faces toward the surface of the person's body.

In an example, a haptic (e.g. haptic and/or tactile) sensation can comprise a selected sequential pattern of movements or vibrations of haptic components on a wearable device. In an example, a system for reducing a person's consumption of unhealthy food can comprise one or more vibrating protrusions on the side of a smart watch which faces toward the surface of the person's body. In an example, a haptic (e.g. haptic and/or tactile) sensation to discourage consumption of unhealthy food can be a specific sequence of vibrations, wherein the rate of vibrations is based in part on the type of unhealthy food detected. In an example, a haptic (e.g. haptic and/or tactile) sensation to discourage consumption of unhealthy food can be a vibration, wherein the frequency and/or rate of the vibration is based in part on the quantity of unhealthy food detected.

In an example, a vibrating or otherwise moving component for providing a haptic and/or tactile sensation can be part of, or attached to, an ear bud. In an example, a vibrating or otherwise moving component for providing a haptic and/or tactile sensation can be part of, or attached to, a wrist band. In an example, a vibrating or otherwise moving component for providing a haptic and/or tactile sensation can be part of, or attached to, a smart collar. In an example, a vibrating or otherwise moving component for providing a haptic and/or tactile sensation can be part of, or attached to, a finger ring. In an example, a method for reducing a person's consumption of unhealthy food can include using a vibrating wrist band or bracelet to provide haptic (e.g. haptic and/or tactile) sensations to a person to reduce the person's consumption of unhealthy food.

In an example, the type and/or pattern of vibration provided to a person by a system for reducing a person's consumption of unhealthy food can be based on the type of food detected in an image. In an example, a haptic (e.g. haptic and/or tactile) sensation can comprise a selected a sequential pattern of component movements or vibrations around (a portion of) a watch band, wrist band, and/or bracelet, wherein the pattern is based on the type and/or quantity of unhealthy food near a person (e.g. detected via analysis of images) and/or unhealthy food consumed by the person (e.g. detected via a chewing sensor and/or analysis of images). In an example, a haptic (e.g. haptic and/or tactile) sensation to discourage consumption of unhealthy food can be a vibration, wherein the magnitude and/or intensity of the vibration is based in part on the quantity of unhealthy food detected.

In an example, a system for reducing a person's consumption of unhealthy food can include a smart watch, watch band, or wrist band which vibrates, wherein the magnitude, frequency, and/or pattern of vibration is based on the type and/or quantity of unhealthy food that is detected as being nearby and/or having been consumed by the person. In an example, the magnitude of a vibration provided to a person by a system for reducing a person's consumption of unhealthy food can be proportional to how unhealthy food in an image would be for that person to eat. In an example, a haptic (e.g. haptic and/or tactile) sensation provided by a system for a person can an array of vibrating components which are worn by the person.

In an example, a haptic (e.g. haptic and/or tactile) sensation can be provided by the vibration or other movement of a selected subset of an array (e.g. array, matrix, or grid) of individually-movable protrusions (e.g. bumps, dots, teeth, pins, or legs) which extend out from a wearable device toward a person's body, wherein the subset is selected by machine learning and/or artificial intelligence based on the type and/or quantity of food nearby and/or consumed by the person. In an example, a haptic (e.g. haptic and/or tactile) sensation can be provided by the vibration or other movement of a selected subset of a row-and-ring array of individually-movable protrusions (e.g. bumps, dots, teeth, pins, or legs) which extend out from a wearable device toward a person's body, wherein the subset is selected by machine learning and/or artificial intelligence based on the type and/or quantity of food nearby and/or consumed by the person.

In an example, a haptic (e.g. haptic and/or tactile) component which provides a haptic (e.g. haptic and/or tactile) sensation can be an array of vibrating components (e.g. a vibrating protrusions, bumps, dots, pins, prongs, or teeth). In an example, a device worn on a person's wrist and/or arm can provide haptic (e.g. haptic and/or tactile) sensations in response to detection of unhealthy food near the person and/or unhealthy food consumed by the person, wherein this device includes an arcuate array of haptic actuators (e.g. vibrating and/or moving components) which span the circumference of the person's wrist and/or arm. In an example, a haptic (e.g. haptic and/or tactile) sensation provided by a system for a person can a circumferential array of vibrating components which are worn around a person's wrist and/or arm.

In an example, a system for reducing a person's consumption of unhealthy food can comprise a necklace, pendant, or collar with a camera to record food images. In an example, a system for reducing a person's consumption of unhealthy food can comprise an earring with a camera to record food images. In an example, a system for reducing a person's consumption of unhealthy food can include a wrist band or bracelet which is used to provide haptic (e.g. haptic and/or tactile) sensations to a person to reduce the person's consumption of unhealthy food. In an example, a wearable camera for detection of nearby unhealthy food can be part of, or attached to, an ear bud. In an example, a wearable camera for detection of nearby unhealthy food can be part of, or attached to, a wrist band.

In an example, a wearable camera for detection of nearby unhealthy food can be part of, or attached to, a smart collar. In an example, a wearable camera for detection of nearby unhealthy food can be part of, or attached to, a finger ring. In an example, a system for reducing a person's consumption of unhealthy food can comprise augmented reality (AR) eyewear. In an example, a system for reducing a person's consumption of unhealthy food can comprise augmented reality (AR) eyewear with at least one camera to record food images. In an example, a wearable camera for detection of nearby unhealthy food can be part of an eyewear attachment. In an example, a method for reducing a person's consumption of unhealthy food can comprise providing haptic (e.g. haptic and/or tactile) sensations in response to unhealthy food via haptic (e.g. haptic and/or tactile) actuators in eyewear (e.g. eyeglasses).

In an example, a system for reducing a person's consumption of unhealthy food can include a chewing sensor which is attached to the central third of the length of the sidepiece (e.g. temple) of eyeglasses worn by the person. In an example, a wearable camera can be attached to conventional eyewear (e.g. eyeglasses). In an example, an imaging device (e.g. camera) can be a modular component which is removably-attached to the front piece of eyewear (e.g. eyeglasses) worn by the person. In an example, an imaging device (e.g. camera) can be part of, or attached to, the sidepiece (e.g. temple) of eyewear (e.g. eyeglasses) worn by the person. In an example, an imaging device (e.g. camera) can be part of, or attached to, the front piece of augmented reality eyewear (e.g. AR eyeglasses) worn by the person. In an example, a vibrating or otherwise moving component for providing a haptic and/or tactile sensation can be part of, or attached to, smart eyewear.

In an example, a system for reducing a person's consumption of unhealthy food can track hand motions and trigger a haptic (e.g. haptic and/or tactile) sensation if a person's hand motions relative to unhealthy food indicate that the person is eating (or intended to eat) the unhealthy food, wherein these hand motions can be selected from the group consisting of: bringing unhealthy food up to their mouth, grasping unhealthy food, reaching toward unhealthy food, and using a utensil to engage (e.g. cut or lift) unhealthy food. In an example, an imaging device (e.g. camera) on can automatically scan space around (e.g. within 12 inches) a person's hands in order to identify food near the person. In an example, food near a person can be defined as food within a 3-foot radius of the person's hands.

In an example, a device worn on a person's wrist and/or arm to provide haptic (e.g. haptic and/or tactile) sensations in response to detection of unhealthy food near the person and/or unhealthy food consumed by the person can be worn on the arm that the person primarily uses to bring food up to their mouth (e.g. the person's dominant arm). In an example, a system for reducing a person's consumption of unhealthy food can comprise a wrist band, bracelet, smart watch, and/or watch band with an inertial motion unit (e.g. accelerometer and gyroscope) to track arm and/or hand motions to identify when a person is eating and/or how much they are eating. In an example, a device worn on a person's wrist and/or arm to provide haptic (e.g. haptic and/or tactile) sensations in response to detection of unhealthy food near the person and/or unhealthy food consumed by the person can span the entire circumference of the person's wrist and/or arm.

In an example, the location of haptic (e.g. haptic and/or tactile) sensation provided by a system for reducing a person's consumption of unhealthy food can depend on the quantity of unhealthy food detected (e.g. quantity of unhealthy food detected nearby, detected in proximity to a person's hand, or detected to have been consumed by the person thus far). In an example, the type of haptic (e.g. haptic and/or tactile) sensation provided by a system for reducing a person's consumption of unhealthy food can depend on the type of unhealthy food detected (e.g. type of unhealthy food detected nearby, detected in proximity to a person's hand, or detected to have been consumed by the person thus far). In an example, the magnitude of haptic (e.g. haptic and/or tactile) sensation provided by a system for reducing a person's consumption of unhealthy food can depend on the proximity of unhealthy food to a person's hand.

In an example, the rate and/or frequency of haptic (e.g. haptic and/or tactile) sensation provided by a system for reducing a person's consumption of unhealthy food can depend on the proximity of unhealthy food to a person's hand. In an example, the magnitude of haptic (e.g. haptic and/or tactile) sensation provided by a system for reducing a person's consumption of unhealthy food can be a positive function of (e.g. proportional to) the quantity of unhealthy food detected (e.g. quantity of unhealthy food detected nearby, detected in proximity to a person's hand, or detected to have been consumed by the person thus far).

In an example, a method for reducing a person's consumption of unhealthy food can comprise: using artificial intelligence and/or machine-learning to analyze data from wearable sensors to measure food consumed by a person; using artificial intelligence and/or machine-learning to identify food near the person; and using artificial intelligence and/or machine-learning to selected a haptic (e.g. haptic and/or tactile) sensation which is provided to the person via a wearable device, wherein the haptic (e.g. haptic and/or tactile) sensation is selected based on joint analysis of food consumed by the person and food near the person. In an example, a method for reducing a person's consumption of unhealthy food can comprise: using artificial intelligence and/or machine-learning to analyze images to identify unhealthy food near a person; and using artificial intelligence and/or machine-learning to selected a haptic (e.g. haptic and/or tactile) sensation which is provided to the person based on the unhealthy food identified in the images.

In an example, a method for reducing a person's consumption of unhealthy food can comprise: using artificial intelligence and/or machine-learning to analyze images to identify unhealthy food near a person; and using artificial intelligence and/or machine-learning to selected a haptic (e.g. haptic and/or tactile) sensation which is provided to the person via a wearable device, wherein the haptic (e.g. haptic and/or tactile) sensation is based on the type and/or quantity of unhealthy food identified in the images. In an example, the magnitude of haptic (e.g. haptic and/or tactile) sensation provided by a system for reducing a person's consumption of unhealthy food can depend on the proximity of unhealthy food to a person's body. In an example, the magnitude and/or type of haptic (e.g. haptic and/or tactile) sensation provided to a person can be proportional to how unhealthy food in an image would be that person to eat.

In an example, the frequency or rate of haptic (e.g. haptic and/or tactile) sensations provided to a person can be increased with the quantity of unhealthy food consumed by the person (e.g. analogous to the increased frequency of sounds from a Geiger counter based on the strength of a radioactive source). In an example, the rate and/or frequency of haptic (e.g. haptic and/or tactile) sensation provided by a system for reducing a person's consumption of unhealthy food can depend on the proximity of unhealthy food to a person's body. In an example, the magnitude of a haptic (e.g. haptic and/or tactile) sensation provided to a person can be increased in proportion with the potential harmfulness of nearby food (e.g. analogous to the increased frequency of sounds from a Geiger counter based on the strength of a radioactive source).

In an example, a person wearing a device to provide haptic (e.g. haptic and/or tactile) sensations can adjust the magnitude of the sensations by one or more mechanisms selected from the following group: a voice command, contracting one or more muscles in a selected manner, making a selected hand gesture, pressing a button on the device, tapping the device, and touching or swiping a screen on the device. In an example, a person wearing a system can increase or decrease the magnitude of haptic (e.g. haptic and/or tactile) sensation provided by the system (e.g. for a selected period of time) by pressing a button or touching a screen. In an example, a person wearing a system can turn it off (e.g. for a selected period of time).

In an example, a system for reducing a person's consumption of unhealthy food can have an adjustable sensitivity level, wherein the person wearing the device can adjust criteria for unhealthy food identification which, in turn, adjusts the degree of unhealthiness required to activate the haptic (e.g. haptic and/or tactile) sensation. For example, if a person is at a holiday or other social event in which they wish to temporarily reduce feedback related to unhealthy types or quantities of food, then they can change the sensitivity level of the system. In an example, the sensitivity level of a system to detection of unhealthy food can be adjusted, wherein a first sensitivity level comprises identification of an unhealthy type of food in an image and a second sensitivity level comprises identification of unhealthy quantity of food in an image.

In an example, a system for reducing a person's consumption of unhealthy food can include an eating sensor (e.g. a hand or arm motion sensor) which detects eating, wherein the system triggers a haptic (e.g. haptic and/or tactile) sensation when analysis of data from the eating sensor indicates that the person has eaten an unhealthy amount of food during a selected time period (e.g. during a meal). In an example, food in an image can be identified as unhealthy for a person to eat based on the types of food that the person has already consumed in a recent selected time period (e.g. in the past hour or two). In an example, a method for reducing a person's consumption of unhealthy food can comprise using machine learning and/or artificial intelligence to evaluate whether consumption of food by a person would be unhealthy, wherein this evaluation is based in part on recent food consumption (e.g. during a recent period of time) by the person.

In an example, a system for reducing a person's consumption of unhealthy food can use one or more of the following factors to determine when a person is consuming unhealthy food: ambient sounds (e.g. sounds associated with food consumption); analysis of images (e.g. analysis of images from a wearable camera to recognize food); body posture or configuration (e.g. posture associated with food consumption); body glucose level (e.g. blood glucose level or interstitial glucose level); body sounds (e.g. chewing and/or swallowing sounds associated with food consumption); cellphone activity (e.g. cellphone activity associated with food consumption); day of the week (e.g. day associated with food consumption); geographic location of the person (e.g. location associated with food consumption); hand gestures (e.g. hand gestures associated with food consumption); hand motions (e.g. hand and/or arm motions associated with food consumption); jaw motions (e.g. jaw motions associated with food consumption); recent financial transactions (e.g. transactions associated with food consumption); room location (e.g. room in a building associated with food consumption); spectroscopic analysis (e.g. spectroscopic analysis of nearby food); and time of day (e.g. time of day associated with food consumption).

In an example, a system for reducing a person's consumption of unhealthy food can track (e.g. monitor, measure, and/or analyze) a person's room location (e.g. room in a building associated with food consumption). In an example, a system for reducing a person's consumption of unhealthy food can further comprise an eating sensor (e.g. a hand or arm motion sensor) which detects eating, wherein the system triggers a wearable imaging device (e.g. camera) to start recording images within three minutes of when the person starts eating (unhealthy food). In an example, a wearable imaging device (e.g. camera) can be triggered to start recording images when analysis of data from a sensor which is in electrical communication with a person's gastrointestinal tract indicates that a person is eating.

In an example, a wearable imaging device (e.g. camera) can be triggered to start recording images when analysis of data from an EEG sensor indicates that a person is eating. In an example, a wearable imaging device (e.g. camera) can be triggered to start recording images when analysis of data from a wearable sensor indicates that a person is chewing and/or swallowing. In an example, a wearable imaging device (e.g. camera) can be triggered to start recording images when analysis of data from a sensor which is in electrical communication with a person's stomach indicates that a person is eating. In an example, a wearable imaging device (e.g. camera) can be triggered to start recording images when an EMG sensor detects that a person is eating. In an example, a wearable imaging device (e.g. camera) can be triggered to start recording images when a wearable sensor detects that a person is chewing and/or swallowing.

In an example, a wearable imaging device (e.g. camera) can be triggered to start recording images when a sensor in electrical communication with a person's stomach detects that a person is eating. In an example, a data processing unit can be a local data processor which is part of a wearable component of the system. In another example, a data processing unit can be a remote data processor which is separate from a wearable component of the system, wherein food data from the wearable component of the system is transmitted (e.g. wirelessly and/or via the internet) to the remote data processor. In an example, a system for reducing a person's consumption of unhealthy food can include a data transmitter and a data receiver, wherein these components create electronic communication between wearable components of the system and a remote data processing unit.

In an example, a wearable system can comprise a data processing hub which is worn on one location on a person's body and a plurality of sensors on other locations on the person's body, wherein there is (wireless) electronic communication between the data processing hub and the sensors, and wherein at least one of the sensors is a camera which detects nearby food. In an example, machine learning and/or artificial intelligence can be used to identify which types of haptic (e.g. haptic and/or tactile) sensations are most effective in reducing a specific person's consumption of unhealthy food. In an example, one can: displace to Jul. 17, 2031; take notes; come back; and write it up. In an example, a method for reducing a person's consumption of unhealthy food can include using a contracting wrist band or bracelet to provide haptic (e.g. haptic and/or tactile) sensations to a person to reduce the person's consumption of unhealthy food.

In an example, a method for reducing a person's consumption of unhealthy food can include using a wrist band or bracelet to provide haptic (e.g. haptic and/or tactile) sensations to a person to reduce the person's consumption of unhealthy food. In an example, a method for reducing a person's consumption of unhealthy food can include using machine learning and/or artificial intelligence to provide a person with a first haptic and/or tactile sensation if it is detected that unhealthy food is near the person and to provide the person with a second haptic and/or tactile sensation if it is detected that the person is consuming unhealthy food. In an example, a method for reducing a person's consumption of unhealthy food can include using machine learning and/or artificial intelligence to identify relationships between a person's past consumption of specific types and/or quantities of food and subsequent changes in their biometric parameters (e.g. which indicate that those types and/or quantities of food are unhealthy for that person).

In an example, a method for reducing a person's consumption of unhealthy food can comprise: receiving images of food from an imaging device (e.g. a camera) worn by a person; using artificial intelligence and/or machine-learning to identify unhealthy food in the images; and using artificial intelligence and/or machine-learning to selected a haptic (e.g. haptic and/or tactile) sensation which is provided to the person based on identification of unhealthy food. In an example, a system for reducing a person's consumption of unhealthy food can use machine learning and/or artificial intelligence to record and/or analyze images (e.g. analysis of images from a wearable camera to recognize food). In an example, an imaging device (e.g. camera) on can automatically scan space in front of a person in order to identify food near the person.

In an example, the focal vector of an imaging device (e.g. camera) which is worn by a person can be automatically moved (e.g. by moving a lens or mirror) in order to scan space in front of the person in order to identify nearby food. In an example, a system for reducing a person's consumption of unhealthy food can include a chewing sensor which measures a person's chewing motions by reflecting light off (the surface of) the person's body (e.g. jaw). In an example, a system for reducing a person's consumption of unhealthy food can include a chewing sensor which measures a person's chewing motions by emitting infrared light toward (the surface of) the person's body (e.g. jaw) and receiving this light after it has been reflected from (the surface of) the person's body.

In an example, a system for reducing a person's consumption of unhealthy food can include an inertial motion sensor to detect when the person eats and/or to track how much they eat. In an example, a system for reducing a person's consumption of unhealthy food can include an inertial motion sensor (e.g. hand and/or arm based accelerometer and gyroscope) to detect when the person eats and/or to track how much they eat. In an example, a system for reducing a person's consumption of unhealthy food can track time of day (e.g. time of day associated with food consumption). In an example, a system for reducing a person's consumption of unhealthy food can use machine learning and/or artificial intelligence to track (e.g. monitor, measure, and/or analyze) body glucose level (e.g. blood glucose level or interstitial glucose level).

In an example, a system for reducing a person's consumption of unhealthy food can track (e.g. monitor, measure, and/or analyze) cellphone activity (e.g. cellphone activity associated with food consumption). In an example, a system for reducing a person's consumption of unhealthy food can use machine learning and/or artificial intelligence to track (e.g. monitor, measure, and/or analyze) body sounds (e.g. chewing and/or swallowing sounds associated with food consumption). In an example, a system for reducing a person's consumption of unhealthy food can use machine learning and/or artificial intelligence to track (e.g. monitor, measure, and/or analyze) hand gestures (e.g. hand gestures associated with food consumption).

In an example, a system for reducing a person's consumption of unhealthy food can track (e.g. monitor, measure, and/or analyze ambient sounds (e.g. sounds associated with food consumption). In an example, a system for reducing a person's consumption of unhealthy food can track (e.g. monitor, measure, and/or analyze) day of the week (e.g. day associated with food consumption). In an example, a system for reducing a person's consumption of unhealthy food can track (e.g. monitor, measure, and/or analyze) hand motions (e.g. hand and/or arm motions associated with food consumption). In an example, a system for reducing a person's consumption of unhealthy food can use machine learning and/or artificial intelligence to perform spectroscopic analysis (e.g. spectroscopic analysis of nearby food).

In an example, a system for reducing a person's consumption of unhealthy food can include a spectroscopy sensor for identification of the (molecular and/or nutritional) composition of nearby food. In an example, food can be identified as unhealthy because it has a high percentage and/or quantity of allergens. In an example, food in an image can be identified as unhealthy for a person to eat based on the values of one or more biometric parameters (e.g. blood glucose level, blood pressure level, etc.) for the person. In an example, food of a particular type and/or quantity can be identified as unhealthy based on its past effects on a person's health as measured by effects on a person's biometric parameters when the person previously consumed food of that type and/or quantity.

In an example, food of a particular type can be identified as unhealthy because it has a high percentage and/or quantity of sugar. In an example, a device worn on a person's wrist and/or arm to provide haptic (e.g. haptic and/or tactile) sensations in response to detection of unhealthy food near the person and/or unhealthy food consumed by the person can be worn on arm opposite the arm that the person primarily uses to bring food up to their mouth (e.g. opposite the person's dominant arm). In an example, a haptic (e.g. haptic and/or tactile) sensation to discourage consumption of unhealthy food can be pressure applied to a person's skin. In an example, a haptic (e.g. haptic and/or tactile) component which provides a haptic (e.g. haptic and/or tactile) sensation can be an electrical energy emitter which transmits a low electrical current to a person's body.

In an example, a haptic (e.g. haptic and/or tactile) sensation can be accompanied by a visual signal. In an example, a system for reducing a person's consumption of unhealthy food can include haptic (e.g. haptic and/or tactile) actuators which are embedded in (e.g. woven or otherwise integrated into) an article of clothing. In an example, a system for reducing a person's consumption of unhealthy food can provide haptic (e.g. haptic and/or tactile) sensations to the person in response to unhealthy food via haptic (e.g. haptic and/or tactile) actuators in on adhesive patch. In an example, a system for reducing a person's consumption of unhealthy food can provide haptic (e.g. haptic and/or tactile) sensations to the person in response to unhealthy food via haptic (e.g. haptic and/or tactile) actuators in a smart watch.

In an example, a system for reducing a person's consumption of unhealthy food can include haptic (e.g. haptic and/or tactile) actuators in a finger ring. In an example, a system for reducing a person's consumption of unhealthy food can provide haptic (e.g. haptic and/or tactile) sensations to the person in response to unhealthy food via haptic (e.g. haptic and/or tactile) actuators in a belt which is worn around a person's waist. In an example, a method for reducing a person's consumption of unhealthy food can comprise providing haptic (e.g. haptic and/or tactile) sensations in response to unhealthy food via haptic (e.g. haptic and/or tactile) actuators which are embedded in (e.g. woven or otherwise integrated into) the cuff and/or sleave of a shirt. In an example, a method for reducing a person's consumption of unhealthy food can comprise providing haptic (e.g. haptic and/or tactile) sensations in response to unhealthy food via haptic (e.g. haptic and/or tactile) actuators in a smart watch.

In an example, a method for reducing a person's consumption of unhealthy food can comprise providing haptic (e.g. haptic and/or tactile) sensations in response to unhealthy food via haptic (e.g. haptic and/or tactile) actuators in a belt which is worn around a person's waist. In an example, a system for reducing a person's consumption of unhealthy food can include one or more moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are moved (e.g. protruded) in a direction which is substantially perpendicular to the surface of the person's body.

In an example, a system for reducing a person's consumption of unhealthy food can comprise one or more moving protrusions on the side of a watch band, wrist band, or bracelet which faces toward the surface of the person's body. In an example, a system for reducing a person's consumption of unhealthy food can comprise one or more moving protrusions on the side of a smart watch which faces toward the surface of the person's body. In an example, a haptic (e.g. haptic and/or tactile) sensation can be provided by movement of a plurality of telescoping protrusions which extend out from a wearable device toward a person's body.

In an example, a system for reducing a person's consumption of unhealthy food can comprise: at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; at least one food consumption sensor (e.g. sound-based eating sensor, light-based/optical eating sensor, muscle/EMG-based eating sensor, brainwave/EEG-based eating sensor, jaw-motion-based eating sensor, body glucose sensor, vibration-based eating sensor, wrist-motion-based eating sensor, hand-gesture-based eating sensor, and/or image-based eating sensor) on the at least one wearable device, wherein data from the food consumption sensor is analyzed (e.g. by machine learning and/artificial intelligence) to detect the types and/or quantities of food (or specific nutrients in food) consumed by the person (e.g. consumed during a period of time); and at least one haptic (e.g. haptic and/or tactile) component (e.g. vibrating or shaking component, component with moving protrusions or sections, or electrical energy emitter) on the one or more wearable devices, wherein the haptic component provides haptic sensations for the person when analysis of data from the one or more food consumption sensors detects consumption of unhealthy types and/or quantities of food by the person.

In an example, haptic and/or tactile sensations in response to unhealthy food (detected nearby and/or consumed by a person) can be provided to the person by an array of piezoelectric actuators on a wearable device which is worn by the person. In an example, a system for reducing a person's consumption of unhealthy food can include an arcuate array of moving components (e.g. protrusions, bumps, rollers, pins, or teeth) on a wearable device which provide the person wearing the device with a haptic (e.g. haptic and/or tactile) sensation through movement of the components in response to nearby and/or consumed unhealthy food, wherein the moving components are located on the side of the device which faces the surface of the person's body.

In an example, a system for reducing a person's consumption of unhealthy food can include an array of individually-movable components (e.g. protrusions, bumps, rollers, pins, or teeth) on a wearable device which provide the person wearing the device with a haptic (e.g. haptic and/or tactile) sensation through movement of the components in response to detection of unhealthy food near the person and/or consumed by the person, wherein movement of a subset of components forms specific haptic (e.g. haptic and/or tactile) patterns (e.g. analogous to patterns of Braille dots) based on the types and/or quantities of unhealthy food detected.

In an example, a method for reducing a person's consumption of unhealthy food can comprise: analyzing images from a camera (e.g. imaging device) on a wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) on a device worn by a person in order to detect types and/or quantities of nearby food; analyzing data from a food consumption sensor (e.g. light-based eating sensor, sound-based eating sensor, EMG-based eating sensor, EEG-based eating sensor, jaw-motion-based eating sensor, body glucose sensor, vibration-based eating sensor, wrist-motion-based eating sensor, hand-gesture-based eating sensor, and/or image-based eating sensor) on a device worn by the person to detect the types and/or quantities of food (or specific nutrients in food) consumed by the person (e.g. consumed during a period of time); and providing haptic (e.g. haptic and/or tactile) sensations for the person via at least one haptic (e.g. haptic and/or tactile) component (e.g. vibrating or shaking component, component with moving protrusions or sections, or electrical energy emitter) on a device worn by the person when analysis of the images detects unhealthy types and/or quantities of food near the person; and/or when analysis of data from the one or more food consumption sensors detects consumption of unhealthy types and/or quantities of food by the person.

In an example, a method for reducing a person's consumption of unhealthy food can comprise: receiving images from at least one camera (e.g. imaging device) on at least one wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) worn by a person; analyzing the images (e.g. using machine learning and/or artificial intelligence) to detect types and/or quantities of nearby food; receiving data from at least one food consumption sensor (e.g. light-based eating sensor, sound-based eating sensor, EMG-based eating sensor, EEG-based eating sensor, jaw-motion-based eating sensor, body glucose sensor, vibration-based eating sensor, wrist-motion-based eating sensor, hand-gesture-based eating sensor, and/or image-based eating sensor) on the at least one wearable device; analyzing the data from the food consumption sensor to detect the types and/or quantities of food (or specific nutrients in food) consumed by the person (e.g. consumed during a period of time); and providing haptic (e.g. haptic and/or tactile) sensations for the person via at least one haptic (e.g. haptic and/or tactile) component (e.g. vibrating or shaking component, component with moving protrusions or sections, or electrical energy emitter) on the one or more wearable devices when analysis of the images detects unhealthy types and/or quantities of food near the person; and/or when analysis of data from the one or more food consumption sensors detects consumption of unhealthy types and/or quantities of food by the person.

In an example, a method for reducing a person's consumption of unhealthy food can comprise: using machine learning and/or artificial intelligence to analyze images from a camera (e.g. imaging device) on a wearable device (e.g. smart eyewear, eyewear attachment, AR eyewear, smart watch, watch band, wrist band, bracelet, arm band, finger ring, smart clothing, adhesive patch, clothing button, external drug pump, ear bud, earring, headband, necklace or pendant, smart belt, and smart collar) on a device worn by a person in order to detect types and/or quantities of nearby food; using machine learning and/or artificial intelligence to analyze data from a food consumption sensor (e.g. light-based eating sensor, sound-based eating sensor, EMG-based eating sensor, EEG-based eating sensor, jaw-motion-based eating sensor, body glucose sensor, vibration-based eating sensor, wrist-motion-based eating sensor, hand-gesture-based eating sensor, and/or image-based eating sensor) on a device worn by the person to detect the types and/or quantities of food (or specific nutrients in food) consumed by the person (e.g. consumed during a period of time); and providing haptic (e.g. haptic and/or tactile) sensations for the person via at least one haptic (e.g. haptic and/or tactile) component (e.g. vibrating or shaking component, component with moving protrusions or sections, or electrical energy emitter) on a device worn by the person when analysis of the images detects unhealthy types and/or quantities of food near the person; and/or when analysis of data from the one or more food consumption sensors detects consumption of unhealthy types and/or quantities of food by the person.

In an example, a haptic (e.g. haptic and/or tactile) sensation provided by a system for reducing a person's consumption of unhealthy food can comprise a sequence of vibrations. In an example, a haptic (e.g. haptic and/or tactile) sensation to discourage consumption of unhealthy food can be a specific pattern of vibration. In an example, a haptic (e.g. haptic and/or tactile) sensation to discourage consumption of unhealthy food can be a vibration, wherein the duration and/or length of the vibration is based in part on the type of unhealthy food detected. In an example, a system for reducing a person's consumption of unhealthy food can include a vibrating component on the side of a smart watch which faces toward the surface of the person's body.

In an example, a system for reducing a person's consumption of unhealthy food can comprise: an imaging device (e.g. a camera) which is worn by a person, wherein the camera captures food images of nearby food; a data processing unit which analyzes the food images to determine whether food in the images is unhealthy for the person to eat; and a haptic (e.g. haptic and/or tactile) actuator (e.g. a vibrating and/or moving device) which is worn by the person, wherein the haptic (e.g. haptic and/or tactile) actuator provides a haptic (e.g. haptic and/or tactile) sensation to the person when food in the images is unhealthy for the person to eat.

In an example, a system for reducing a person's consumption of unhealthy food can provide a haptic (e.g. haptic and/or tactile) sensor to a person by one or more mechanisms selected from the group consisting of: a low-current electrical transmitting which is in contact with the person's skin; a moving component which is in contact with the person's skin; a moving component which taps the person's skin; a neurostimulator which is contact with the person's skin; a piezoelectric component which is in contact with the person's skin; and a vibrating protrusion which is in contact with the person's skin. In an example, a haptic (e.g. haptic and/or tactile) sensation to discourage consumption of unhealthy food can be a specific sequence of vibrations, wherein the rate of vibrations is based in part on the quantity of unhealthy food detected.

In an example, a vibrating or otherwise moving component for providing a haptic and/or tactile sensation can be part of, or attached to, smart clothing. In an example, a vibrating or otherwise moving component for providing a haptic and/or tactile sensation can be part of, or attached to, an arm band. In an example, a vibrating or otherwise moving component for providing a haptic and/or tactile sensation can be part of, or attached to, a watch band. In an example, a vibrating or otherwise moving component for providing a haptic and/or tactile sensation can be part of, or attached to, a smart belt. In an example, a vibrating or otherwise moving component for providing a haptic and/or tactile sensation can be part of, or attached to, a clothing button. In an example, a method for reducing a person's consumption of unhealthy food can include using a vibrating watch band to provide haptic (e.g. haptic and/or tactile) sensations to a person to reduce the person's consumption of unhealthy food.

In an example, a haptic (e.g. haptic and/or tactile) sensation can comprise a selected sequential pattern of component movements or vibrations, wherein the pattern is based on the type and/or quantity of unhealthy food near a person (e.g. detected via analysis of images) and/or unhealthy food consumed by the person (e.g. detected via a chewing sensor and/or analysis of images). In an example, a haptic (e.g. haptic and/or tactile) sensation can be provided by the vibration or other movement of a selected subset of a plurality of individually-movable protrusions (e.g. bumps, dots, teeth, pins, or legs) which extend out from a wearable device toward a person's body, wherein the subset is selected by machine learning and/or artificial intelligence based on the type and/or quantity of food nearby and/or consumed by the person.

In an example, the magnitude of a vibration provided to a person by a system for reducing a person's consumption of unhealthy food can be a positive function of how unhealthy food in an image would be for that person to eat. In an example, a system for reducing a person's consumption of unhealthy food can include an arm band or smart belt which vibrates, wherein the magnitude, frequency, and/or pattern of vibration is based on the type and/or quantity of unhealthy food that is detected as being nearby and/or having been consumed by the person. In an example, a haptic (e.g. haptic and/or tactile) sensation provided by a system for a person can an array or matrix of vibrating components which is worn by a person. In an example, haptic and/or tactile sensations in response to unhealthy food (detected nearby and/or consumed by a person) can be provided to the person by an array of vibrating components on a wearable device which is worn by the person.

In an example, a haptic (e.g. haptic and/or tactile) sensation can be provided by the vibration or other movement of a selected subset of an array (e.g. array, matrix, or grid) of individually-movable protrusions (e.g. bumps, dots, teeth, pins, or legs) which extend out from a wearable device toward a person's body, wherein the subset is selected by machine learning and/or artificial intelligence based on the type and/or quantity of food nearby and/or consumed by the person. In an example, a haptic (e.g. haptic and/or tactile) sensation can be provided by the vibration or other movement of a selected subset of a row-and-column (e.g. orthogonal) array of individually-movable protrusions (e.g. bumps, dots, teeth, pins, or legs) which extend out from a wearable device toward a person's body, wherein the subset is selected by machine learning and/or artificial intelligence based on the type and/or quantity of food nearby and/or consumed by the person.

In an example, a haptic (e.g. haptic and/or tactile) sensation can comprise a selected a sequential pattern of component movements or vibrations around (a portion of) the circumference of a person's wrist or arm, wherein the pattern is based on the type and/or quantity of unhealthy food near a person (e.g. detected via analysis of images) and/or unhealthy food consumed by the person (e.g. detected via a chewing sensor and/or analysis of images). In an example, a device worn on a person's wrist and/or arm to provide haptic (e.g. haptic and/or tactile) sensations in response to detection of unhealthy food near the person and/or unhealthy food consumed by the person, wherein this device includes an arcuate array of haptic actuators (e.g. vibrating and/or moving components) which span the circumference of the person's wrist and/or arm, wherein these haptic actuators are actuated in a radial sequential pattern around the circumference of the person's wrist and/or arm.

In an example, a haptic (e.g. haptic and/or tactile) component which provides a haptic (e.g. haptic and/or tactile) sensation can be a circumferential (e.g. circular or elliptical) array of vibrating components (e.g. a vibrating protrusions, bumps, dots, pins, prongs, or teeth). In an example, a system for reducing a person's consumption of unhealthy food can comprise a wrist band, bracelet, smart watch, and/or watch band with a camera to record food images. In an example, a system for reducing a person's consumption of unhealthy food can include a smart watch which is used to provide haptic (e.g. haptic and/or tactile) sensations to a person to reduce the person's consumption of unhealthy food. In an example, a wearable camera for detection of nearby unhealthy food can be part of, or attached to, smart clothing.

In an example, a wearable camera for detection of nearby unhealthy food can be part of, or attached to, an arm band. In an example, a wearable camera for detection of nearby unhealthy food can be part of, or attached to, a watch band. In an example, a wearable camera for detection of nearby unhealthy food can be part of, or attached to, a smart belt. In an example, a wearable camera for detection of nearby unhealthy food can be part of, or attached to, a clothing button. In an example, a system for reducing a person's consumption of unhealthy food can comprise augmented reality (AR) eyewear with bilateral cameras to record three-dimensional food images.

In an example, a system for reducing a person's consumption of unhealthy food can comprise AR eyewear which provides visual feedback in response to detection of unhealthy food in addition to a wearable device (e.g. a wrist-worn device) which provides haptic (e.g. haptic and/or tactile) sensation in response to detection of unhealthy food. In an example, AR eyewear can change the perceived color of unhealthy food in a person's field of view to make the food less appetizing. In an example, AR eyewear can display virtual objects in proximity to unhealthy food in a person's field of view to make the food less appetizing. In an example, a system for reducing a person's consumption of unhealthy food can provide haptic (e.g. haptic and/or tactile) sensations to the person in response to unhealthy food via haptic (e.g. haptic and/or tactile) actuators on eyewear (e.g. eyeglasses).

In an example, a vibrating or otherwise moving component for providing a haptic and/or tactile sensation can be part of an eyewear attachment. In an example, a system for reducing a person's consumption of unhealthy food can include a chewing sensor and a camera which are attached to the central third of the length of the sidepiece (e.g. temple) of eyeglasses worn by the person. In an example, a wearable camera for detection of nearby unhealthy food can be part of, or attached to, smart eyewear. In an example, an imaging device (e.g. camera) can be a modular component which is removably-attached to sidepiece (e.g. temple) of eyewear (e.g. eyeglasses) worn by the person. In an example, an imaging device (e.g. camera) can be part of, or attached to, the sidepiece (e.g. temple) of augmented reality eyewear (e.g. AR eyeglasses) worn by the person. In an example, an imaging device (e.g. camera) can be part of, or attached to, eyewear (e.g. eyeglasses) worn by the person.

In an example, a vibrating or otherwise moving component for providing a haptic and/or tactile sensation can be part of, or attached to, AR eyewear. In an example, a system for reducing a person's consumption of unhealthy food can track a person's hand in order to detect one or more hand motions selected from the group consisting of: bringing unhealthy food up to their mouth, grasping unhealthy food, reaching toward unhealthy food, and using a utensil to engage (e.g. cut or lift) unhealthy food. In an example, an imaging device (e.g. camera) on can automatically scan space around (e.g. within 6 inches) a person's hands in order to identify food near the person.

In an example, the focal vector of an imaging device (e.g. camera) which is worn by a person can track the location of a person's hand (or hands) in order to identify nearby food by hand-to-food interactions. In an example, a person can wear two devices, one on each wrist and/or arm, to provide haptic (e.g. haptic and/or tactile) sensations in response to detection of unhealthy food near the person and/or unhealthy food consumed by the person. In an example, a system for reducing a person's consumption of unhealthy food can track arm and/or hand motions and/or gestures to identify when a person is eating and/or how much they are eating.

In an example, a device worn on a person's wrist and/or arm to provide haptic (e.g. haptic and/or tactile) sensations in response to detection of unhealthy food near the person and/or unhealthy food consumed by the person can span between 60% and 90% of the entire circumference of the person's wrist and/or arm. In an example, the pattern of haptic (e.g. haptic and/or tactile) sensation provided by a system for reducing a person's consumption of unhealthy food can depend on the type of unhealthy food detected (e.g. type of unhealthy food detected nearby, detected in proximity to a person's hand, or detected to have been consumed by the person thus far). In an example, the type of haptic (e.g. haptic and/or tactile) sensation provided by a system for reducing a person's consumption of unhealthy food can depend on the quantity of unhealthy food detected (e.g. quantity of unhealthy food detected nearby, detected in proximity to a person's hand, or detected to have been consumed by the person thus far).

In an example, the magnitude of haptic (e.g. haptic and/or tactile) sensation provided by a system for reducing a person's consumption of unhealthy food can depend on the type of unhealthy food detected (e.g. type of unhealthy food detected nearby, detected in proximity to a person's hand, or detected to have been consumed by the person thus far). In an example, the rate and/or frequency of haptic (e.g. haptic and/or tactile) sensation provided by a system for reducing a person's consumption of unhealthy food can depend on the type of unhealthy food detected (e.g. type of unhealthy food detected nearby, detected in proximity to a person's hand, or detected to have been consumed by the person thus far).

In an example, the magnitude of haptic (e.g. haptic and/or tactile) sensation provided by a system for reducing a person's consumption of unhealthy food can be a positive function of (e.g. proportional to) the level of unhealthiness of unhealthy food detected (e.g. quantity of unhealthy food detected nearby, detected in proximity to a person's hand, or detected to have been consumed by the person thus far). In an example, a method for reducing a person's consumption of unhealthy food can comprise: using artificial intelligence and/or machine-learning to identify unhealthy food near a person in images; and using artificial intelligence and/or machine-learning to selected a haptic (e.g. haptic and/or tactile) sensation which is provided to the person based on the unhealthy food identified in the images.

In an example, a method for reducing a person's consumption of unhealthy food can comprise: using artificial intelligence and/or machine-learning to analyze images to identify unhealthy food near a person; and using artificial intelligence and/or machine-learning to selected a haptic (e.g. haptic and/or tactile) sensation which is provided to the person via a wearable device, wherein the haptic (e.g. haptic and/or tactile) sensation is based on the unhealthy food identified in the images. In an example, the magnitude and/or type of haptic (e.g. haptic and/or tactile) sensation provided to a person can depend on the degree to which consumption of food in an image would be unhealthy for that person.

In an example, a method for reducing a person's consumption of unhealthy food can include: using machine learning and/or artificial intelligence to analyze the effectiveness of haptic (e.g. haptic and/or tactile) sensations on reducing a person's consumption of unhealthy food in the past; and using the results of this analysis to select the type and/or magnitude of haptic (e.g. haptic and/or tactile) sensation provided to the person to reduce consumption of unhealthy food now. In an example, a method for reducing a person's consumption of unhealthy food can comprise using machine learning and/or artificial intelligence to evaluate whether consumption of food by a person would be unhealthy, wherein this evaluation is based in part on current values for one or more of the person's biometric parameters (e.g. glucose level, blood pressure, heart rate, blood alcohol level, etc.).

In an example, the frequency or rate of haptic (e.g. haptic and/or tactile) sensations provided to a person can be increased as a person moves their hand closer to unhealthy food (e.g. analogous to the increased frequency of sounds from a Geiger counter with proximity to a radioactive source). In an example, the magnitude of a haptic (e.g. haptic and/or tactile) sensation provided to a person can be increased as a person moves their hand closer to unhealthy food (e.g. analogous to the increased frequency of sounds from a Geiger counter with proximity to a radioactive source). In an example, a device worn by a person which provides haptic (e.g. haptic and/or tactile) sensations to the person in response to detection of nearby unhealthy food and/or consumption of unhealthy food by the person can adjust the magnitude of the sensations automatically based on the person's environment and/or social context.

In an example, a person wearing a device to provide haptic (e.g. haptic and/or tactile) sensations can adjust the form of the sensations by one or more mechanisms selected from the following group: a voice command, contracting one or more muscles in a selected manner, making a selected hand gesture, pressing a button on the device, tapping the device, and touching or swiping a screen on the device. In an example, a person wearing a system can increase or decrease the sensitivity of the system to detection of unhealthy food (e.g. for a selected period of time). In an example, a person wearing a system can turn it off (e.g. for a selected period of time) by pressing a button or touching a screen.

In an example, machine learning and/or artificial reality can be used to automatically adjust the magnitude or form of haptic (haptic and/or tactile) sensations which are provided to the person based on analysis of the person's environment and/or social context. In an example, the sensitivity level of a system to detection of unhealthy food can be adjusted, wherein a first (higher) sensitivity level comprises identification of any unhealthy food within an image and a second (lower) sensitivity level comprises identification of unhealthy food with which a person is interacting with a hand (e.g. grasping food or bringing food up to their mouth).

In an example, a system for reducing a person's consumption of unhealthy food can further comprise an eating sensor which detects eating, wherein the system triggers a haptic (e.g. haptic and/or tactile) sensation when analysis of data from the eating sensor indicates that the person has eaten an unhealthy amount of food during a selected time period (e.g. during a meal). In an example, food in an image can be identified as unhealthy for a person to eat based on the amount of food that the person has already consumed in a recent selected time period (e.g. in the past hour or two). In an example, a wearable imaging device (e.g. camera) can be triggered to start recording images when analysis of data from a wearable sensor indicates that a person has been eating or drinking for more than a selected period of time (e.g. 3 or 5 minutes).

In an example, a system for reducing a person's consumption of unhealthy food can track (e.g. monitor, measure, and/or analyze one or more factors selected from the group consisting of: ambient sounds (e.g. sounds associated with food consumption); analysis of images (e.g. analysis of images from a wearable camera to recognize food); body posture or configuration (e.g. posture associated with food consumption); body glucose level (e.g. blood glucose level or interstitial glucose level); body sounds (e.g. chewing and/or swallowing sounds associated with food consumption); cellphone activity (e.g. cellphone activity associated with food consumption); day of the week (e.g. day associated with food consumption); geographic location of the person (e.g. location associated with food consumption); hand gestures (e.g. hand gestures associated with food consumption); hand motions (e.g. hand and/or arm motions associated with food consumption); jaw motions (e.g. jaw motions associated with food consumption); recent financial transactions (e.g. transactions associated with food consumption); room location (e.g. room in a building associated with food consumption); spectroscopic analysis (e.g. spectroscopic analysis of nearby food); and time of day (e.g. time of day associated with food consumption).

In an example, a system for reducing a person's consumption of unhealthy food can include an eating sensor which detects eating, wherein the system triggers a wearable imaging device (e.g. camera) to start recording images when analysis of data from the eating sensor indicates that the person is eating. In an example, a system for reducing a person's consumption of unhealthy food can further comprise an eating sensor (e.g. a chewing and/or swallowing sensor) which detects eating, wherein the system triggers a wearable imaging device (e.g. camera) to start recording images when analysis of data from the eating detector indicates that the person is eating.

In an example, a wearable imaging device (e.g. camera) can be triggered to start recording images when a sensor in electrical communication with a person's gastrointestinal tract detects that a person is eating. In an example, a wearable imaging device (e.g. camera) can be triggered to start recording images when analysis of data from an EMG sensor indicates that a person is eating. In an example, a wearable imaging device (e.g. camera) can be triggered to start recording images when analysis of data from a wearable sensor indicates that a person is consuming food. In an example, a wearable imaging device (e.g. camera) can be triggered to start recording images when analysis of data from a wearable optical sensor indicates that a person is eating.

In an example, a wearable imaging device (e.g. camera) can be triggered to start recording images when an EEG sensor detects that a person is eating. In an example, a wearable imaging device (e.g. camera) can be triggered to start recording images when a wearable sensor detects that a person is consuming food. In an example, a wearable imaging device (e.g. camera) can be triggered to start recording images when a sensor in a person's mouth detects that a person is eating. In an example, a wearable system can comprise a data processing hub which is worn on one location on a person's body and a plurality of sensors on other locations on the person's body, wherein there is (wireless) electronic communication between the data processing hub and the sensors.

In an example, a system for reducing a person's consumption of unhealthy food can provide a haptic (e.g. haptic and/or tactile) sensation to a person within three minutes of detection of unhealthy food near the person based on analysis of images from a camera worn by the person. In an example, machine learning and/or artificial intelligence can be used to identify which types of haptic (e.g. haptic and/or tactile) sensations are most effective in reducing a specific person's consumption of unhealthy food, wherein identification of the most effective sensations is based on analysis of the relationship between past sensations provided to the person and the person's past consumption of unhealthy food. In an example, machine learning and/or artificial intelligence can analyze haptic (e.g. haptic and/or tactile) sensations provided to a person in the past and unhealthy food consumed by that person in the past in order to identify which types of haptic (e.g. haptic and/or tactile) sensations are most effective in reducing that person's consumption of unhealthy food.

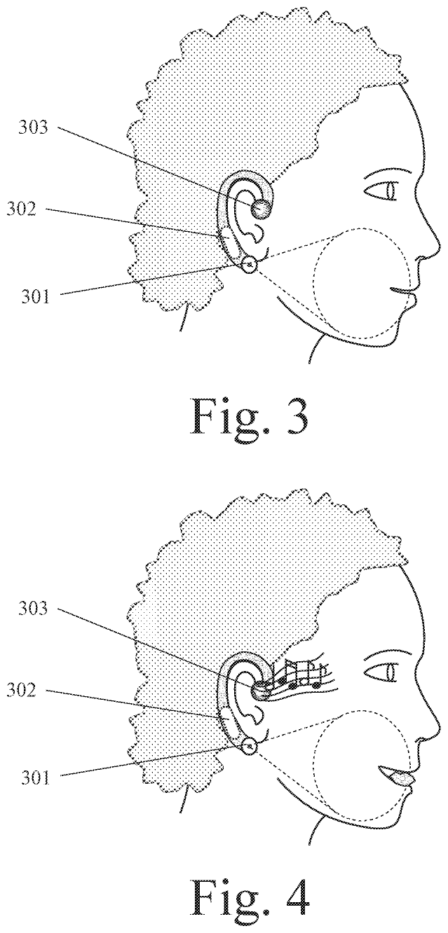

FIGS. 3 and 4 show views, at two different times, of a wearable system and/or device for modifying a person's food consumption comprising: (a) an eating detector 301 which is worn by a person, wherein data from the eating detector is analyzed to identify food consumption by the person; (b) one or more energy emitters 303 which are worn by the person, wherein patterns of energy (e.g. music or sound tones) emitted from the one or more energy emitters are selected to change the person's food consumption in a desired manner (e.g. to reduce the person's consumption of an unhealthy quantity and/or type of food); and (c) a data processor 302, wherein data from the eating detector is analyzed in the data processor, and wherein selection of the patterns of energy to be emitted from the one or more energy emitters is based in part on analysis of current data from the eating detector and/or based in part on historical associations between patterns of energy emitted from the one or more energy emitters and subsequent changes in the person's food consumption. FIG. 3 shows this system at a first time, before the person has started eating. FIG. 4 shows this system at a second time, when the person has started eating. In FIG. 4, food consumption has been analyzed based on data from the eating detector and an energy emitter has been activated to emit music or sound tones to change the person's food consumption in a desired manner.

In this example, the eating detector is an optical sensor (e.g. infrared sensor) which detects movement of the person's muscles and/or jaw which indicates food consumption (e.g. chewing and/or swallowing). In another example, an eating detector can be an inertial motion sensor (e.g. accelerometer) which detects movement of the person's muscles and/or jaw which indicates food consumption (e.g. chewing and/or swallowing). In another example, an eating detector can be an inertial motion sensor (e.g. accelerometer) which detects movement of the person's hand to their mouth in a manner which indicates food consumption (e.g. upward hand movement, wrist rotation, pause, and then downward wrist movement).

In another example, an eating detector can be a sound sensor (e.g. microphone) which records sounds which indicate food consumption (e.g. chewing and/or swallowing). In another example, an eating detector can be an imaging sensor (e.g. camera) which records images which indicate food consumption (e.g. images of food being consumed). In another example, an eating detector can be an electromagnetic energy sensor (e.g. EMG sensor or EEG sensor) which records electromagnetic patterns from muscles or the brain which indicate food consumption.

In another example, an eating detector can comprise two types of sensors, wherein a primary sensor is active continually and a secondary sensor which is only activated (e.g. triggered) when the primary sensor indicates food consumption. In an example, the primary sensor can be less intrusive of privacy than the secondary sensor so a hierarchical two-sensor system helps to maintain privacy. In an example, the primary sensor can require less power to operate than the secondary sensor so a hierarchical two-sensor system helps to reduce the power requirement of the system. In an example, the primary sensor can be less accurate for identifying the quantities and types of food consumed than the secondary sensor. In an example, the primary sensor can be a non-imaging optical sensor and the secondary sensor can be an image-based sensor. In an example, the primary sensor can be a motion sensor and the secondary sensor can be an image-based sensor.

In this example, the eating detector and the energy emitter are both incorporated into an ear-worn device (e.g. ear bud, ear loop, or ear ring). In an example, an eating detector and/or an energy emitter can be incorporated into specialized eyewear (e.g. AR/VR eyeglasses). In an example, an eating detector and/or an energy emitter can be removably attached to the frame of conventional eyeglasses. In an example, an eating detector and/or an energy emitter can be incorporated into a necklace and/or pendant. In an example, an eating detector and/or an energy emitter can be incorporated into a headband. In an example, an eating detector and/or an energy emitter can be incorporated into a wearable patch or tattoo. In an example, an eating detector and/or an energy emitter can be incorporated into a wrist-worn device (e.g. smart watch, watch band, wrist band, or bracelet).

In this example, the energy emitter is a sonic energy emitter. In this example, the energy emitter is a speaker. In this example, the energy pattern plays music and/or a sequence of sounds or tones. In an example, this system can play a selected type or genre of music (or sound tones) when analysis of data from the eating detector indicates that a person is consuming a selected quantity or type of food. In an example, the selected music or sounds can be documented as helping to modify the person's food consumption in the past. In an example, this system can play soothing and/or calming music (or sound tones) when a person is consuming an unhealthy quantity or type of food in order to reduce the person's consumption of the unhealthy quantity or type of food. In an example, the tempo of music (or sound tone patterns) played can be selected to encourage the person to eat more slowly.

In an example, a system can further comprise one or more biometric sensors which are used to detect when the person is emotionally stressed, wherein the system can play a selected type or genre of music to reduce unhealthy emotion-based food consumption when the person is emotionally stressed. In an example, a system can further comprise biometric sensors which track a person's blood pressure, pulse rate, and/or other biometric parameters to detect when the person is emotionally stressed, wherein the system can play a selected type or genre of music to reduce unhealthy emotion-based food consumption when the person is emotionally stressed. In an example, this system can provide real-time music therapy to help a person to avoid unhealthy, emotion-drive food consumption. In an example, this system can play binaural sound patterns to entrain the person's brainwaves, which may help the person to be less anxious and eat less.

In this example, the data processor which analyzes data from the eating detector and selects the pattern of energy to be emitted by an energy emitter is part of a wearable device (e.g. ear-worn device). In another example, a data processor which analyzes data from an eating detector and selects the pattern of energy to be emitted by an energy emitter can a remote data processor with which a local data processor (and data transmitter) can be in electronic communication. In an example, a remote data processor can be located in “the cloud” (a remote server) with which a local data processor is in electronic communication via the internet. In an example, this system can further comprise a wireless data transmitter, a wireless data receiver, and a power supply (e.g. battery).

In an example, this system can further comprise an electronic data file which includes historical information on food consumption and historical information on emitted energy patterns. This information is analyzed to identify which specific energy patterns have been most effective in modifying subsequent food consumption in a desired manner. In an example, this analysis of historical information can be used to help select which energy patterns are selected to be emitted by the system to modify which types of food consumption. In an example, a particular type or genre of music may be most effective at reducing emotion-based food consumption. Different types or genres of music may be most effective for different people. The most effective music for a particular person can be identified by analyzing historical food consumption information for that specific person.

For example, for some people a particular country music song may suddenly increase their desire for Tennessee Whisky. However, for other people, that same song may remind them why their spouse left, thereby decreasing their desire for Tennessee Whisky. In another example, for some people new age music may give them the munchies. However, for other people new age music may cause then to be nauseas and completely takes away any appetite. The effects of the “Oompa Loompa” song from Willy Wonka on consumption of chocolate can also be highly variable by individual. Example variations discussed elsewhere in this disclosure can also be applied to this example where relevant.

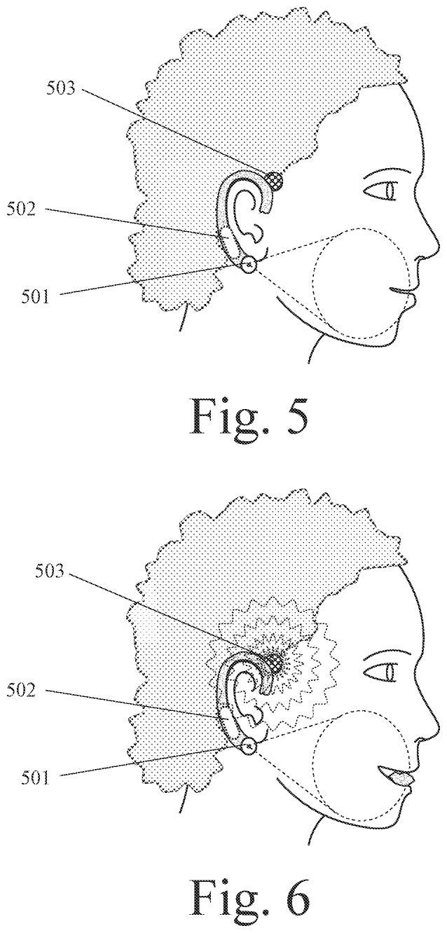

FIGS. 5 and 6 show views, at two different times, of a wearable system and/or device for modifying a person's food consumption comprising: (a) an eating detector 501 which is worn by a person, wherein data from the eating detector is analyzed to identify food consumption by the person; (b) one or more energy emitters 503 which are worn by the person, wherein patterns of energy (e.g. neurostimulation) emitted from the one or more energy emitters are selected to change the person's food consumption in a desired manner (e.g. to reduce the person's consumption of an unhealthy quantity and/or type of food); and (c) a data processor 502, wherein data from the eating detector is analyzed in the data processor, and wherein selection of the patterns of energy to be emitted from the one or more energy emitters is based in part on analysis of current data from the eating detector and/or based in part on historical associations between patterns of energy emitted from the one or more energy emitters and subsequent changes in the person's food consumption. FIG. 5 shows this system at a first time, before the person has started eating. FIG. 6 shows this system at a second time, when the person has started eating. In FIG. 6, food consumption has been analyzed based on data from the eating detector and an energy emitter has been activated to emit neurostimulation to change the person's food consumption in a desired manner.

In this example, the eating detector is an optical sensor (e.g. infrared sensor) which detects movement of the person's muscles and/or jaw which indicates food consumption (e.g. chewing and/or swallowing). In another example, an eating detector can be an inertial motion sensor (e.g. accelerometer) which detects movement of the person's muscles and/or jaw which indicates food consumption (e.g. chewing and/or swallowing). In another example, an eating detector can be an inertial motion sensor (e.g. accelerometer) which detects movement of the person's hand to their mouth in a manner which indicates food consumption (e.g. upward hand movement, wrist rotation, pause, and then downward wrist movement).

In another example, an eating detector can be a sound sensor (e.g. microphone) which records sounds which indicate food consumption (e.g. chewing and/or swallowing). In another example, an eating detector can be an imaging sensor (e.g. camera) which records images which indicate food consumption (e.g. images of food being consumed). In another example, an eating detector can be an electromagnetic energy sensor (e.g. EMG sensor or EEG sensor) which records electromagnetic patterns from muscles or the brain which indicate food consumption.

In another example, an eating detector can comprise two types of sensors, wherein a primary sensor is active continually and a secondary sensor which is only activated (e.g. triggered) when the primary sensor indicates food consumption. In an example, the primary sensor can be less intrusive of privacy than the secondary sensor so a hierarchical two-sensor system helps to maintain privacy. In an example, the primary sensor can require less power to operate than the secondary sensor so a hierarchical two-sensor system helps to reduce the power requirement of the system. In an example, the primary sensor can be less accurate for identifying the quantities and types of food consumed than the secondary sensor. In an example, the primary sensor can be a non-imaging optical sensor and the secondary sensor can be an image-based sensor. In an example, the primary sensor can be a motion sensor and the secondary sensor can be an image-based sensor.

In this example, the eating detector and the energy emitter are both incorporated into an ear-worn device (e.g. ear bud, ear loop, or ear ring). In an example, an eating detector and/or an energy emitter can be incorporated into specialized eyewear (e.g. AR/VR eyeglasses). In an example, an eating detector and/or an energy emitter can be removably attached to the frame of conventional eyeglasses. In an example, an eating detector and/or an energy emitter can be incorporated into a necklace and/or pendant. In an example, an eating detector and/or an energy emitter can be incorporated into a headband. In an example, an eating detector and/or an energy emitter can be incorporated into a wearable patch or tattoo. In an example, an eating detector and/or an energy emitter can be incorporated into a wrist-worn device (e.g. smart watch, watch band, wrist band, or bracelet).

In this example, the energy emitter is an electrical and/or electromagnetic energy emitter. In this example, the energy emitter is an electrode which emits electrical and/or electromagnetic energy. In this example, the energy emitter is an electrode provides neurostimulation of the brain and/or peripheral nerves, wherein this neurostimulation modifies the person's food consumption. In this example, the energy emitter is an electrode provides neurostimulation of the brain and/or peripheral nerves, wherein this neurostimulation modifies the person's food consumption by reducing hunger.

In another example, an energy emitter can be a speaker. In another example, an energy emitter can play music and/or a sequence of sounds or tones. In another example, this system can play a selected type or genre of music (or sound tones) when analysis of data from the eating detector indicates that a person is consuming a selected quantity or type of food. In another example, the selected music or sounds can be documented as helping to modify the person's food consumption in the past. In another example, this system can play soothing and/or calming music (or sound tones) when a person is consuming an unhealthy quantity or type of food in order to reduce the person's consumption of the unhealthy quantity or type of food. In another example, the tempo of music (or sound tone patterns) played can be selected to encourage the person to eat more slowly.

In an example, a system can further comprise one or more biometric sensors which are used to detect when the person is emotionally stressed and the system can provide a selected type of neurostimulation to reduce unhealthy emotion-based food consumption when the person is emotionally stressed. In an example, a system can further comprise biometric sensors which track a person's blood pressure, pulse rate, and/or other biometric parameters to detect when the person is emotionally stressed, wherein the system can provide a selected pattern of neurostimulation to reduce unhealthy emotion-based food consumption when the person is emotionally stressed. In an example, this neurostimulation can be provided at selected anatomical locations on the surface of the person's head, neck, or torso. In an example, this system can provide real-time neurostimulation therapy to help a person to avoid unhealthy, emotion-drive food consumption.

In this example, the data processor which analyzes data from the eating detector and selects the pattern of energy to be emitted by an energy emitter is part of a wearable device (e.g. ear-worn device). In another example, a data processor which analyzes data from an eating detector and selects the pattern of energy to be emitted by an energy emitter can a remote data processor with which a local data processor (and data transmitter) can be in electronic communication. In an example, a remote data processor can be located in “the cloud” (a remote server) with which a local data processor is in electronic communication via the internet. In an example, this system can further comprise a wireless data transmitter, a wireless data receiver, and a power supply (e.g. battery).

In an example, this system can further comprise an electronic data file which includes historical information on food consumption and historical information on emitted energy patterns. This information is analyzed to identify which specific energy patterns (e.g. neurostimulation patterns) have been most effective in modifying subsequent food consumption in a desired manner. In an example, this analysis of historical information can be used to help select which energy patterns are selected to be emitted by the system to modify which types of food consumption. In an example, a particular pattern of neurostimulation may be most effective at reducing unhealthy food consumption. Example variations discussed elsewhere in this disclosure can also be applied to this example where relevant.

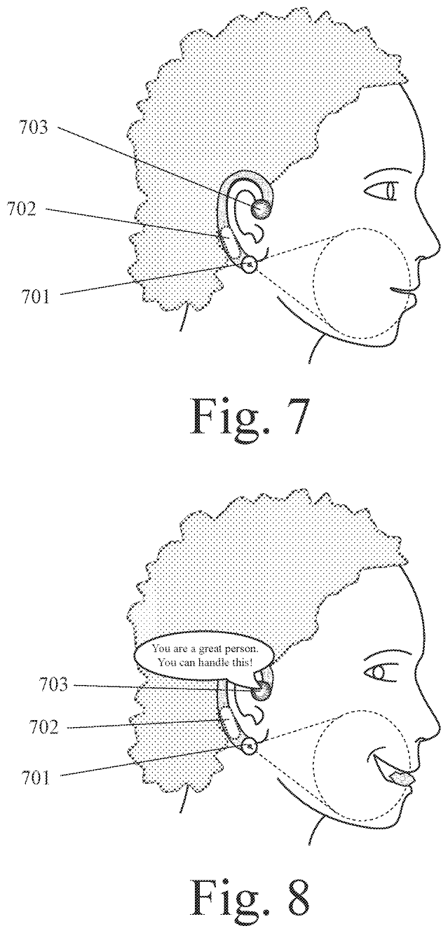

FIGS. 7 and 8 show views, at two different times, of a wearable system and/or device for modifying a person's food consumption comprising: (a) an eating detector 701 which is worn by a person, wherein data from the eating detector is analyzed to identify food consumption by the person; (b) one or more energy emitters 703 which are worn by the person, wherein patterns of energy (e.g. encouraging and/or complimentary spoken words) emitted from the one or more energy emitters are selected to change the person's food consumption in a desired manner (e.g. to reduce the person's consumption of an unhealthy quantity and/or type of food); and (c) a data processor 702, wherein data from the eating detector is analyzed in the data processor, and wherein selection of the patterns of energy to be emitted from the one or more energy emitters is based in part on analysis of current data from the eating detector and/or based in part on historical associations between patterns of energy emitted from the one or more energy emitters and subsequent changes in the person's food consumption. FIG. 7 shows this system at a first time, before the person has started eating. FIG. 8 shows this system at a second time, when the person has started eating. In FIG. 8 food consumption has been analyzed based on data from the eating detector and a speaker has been activated to transmit encouraging and/or complimentary words to change the person's food consumption in a desired manner.

In this example, the eating detector is an optical sensor (e.g. infrared sensor) which detects movement of the person's muscles and/or jaw which indicates food consumption (e.g. chewing and/or swallowing). In another example, an eating detector can be an inertial motion sensor (e.g. accelerometer) which detects movement of the person's muscles and/or jaw which indicates food consumption (e.g. chewing and/or swallowing). In another example, an eating detector can be an inertial motion sensor (e.g. accelerometer) which detects movement of the person's hand to their mouth in a manner which indicates food consumption (e.g. upward hand movement, wrist rotation, pause, and then downward wrist movement).

In another example, an eating detector can be a sound sensor (e.g. microphone) which records sounds which indicate food consumption (e.g. chewing and/or swallowing). In another example, an eating detector can be an imaging sensor (e.g. camera) which records images which indicate food consumption (e.g. images of food being consumed). In another example, an eating detector can be an electromagnetic energy sensor (e.g. EMG sensor or EEG sensor) which records electromagnetic patterns from muscles or the brain which indicate food consumption.

In another example, an eating detector can comprise two types of sensors, wherein a primary sensor is active continually and a secondary sensor which is only activated (e.g. triggered) when the primary sensor indicates food consumption. In an example, the primary sensor can be less intrusive of privacy than the secondary sensor so a hierarchical two-sensor system helps to maintain privacy. In an example, the primary sensor can require less power to operate than the secondary sensor so a hierarchical two-sensor system helps to reduce the power requirement of the system. In an example, the primary sensor can be less accurate for identifying the quantities and types of food consumed than the secondary sensor. In an example, the primary sensor can be a non-imaging optical sensor and the secondary sensor can be an image-based sensor. In an example, the primary sensor can be a motion sensor and the secondary sensor can be an image-based sensor.

In this example, the eating detector and the energy emitter are both incorporated into an ear-worn device (e.g. ear bud, ear loop, or ear ring). In an example, an eating detector and/or an energy emitter can be incorporated into specialized eyewear (e.g. AR/VR eyeglasses). In an example, an eating detector and/or an energy emitter can be removably attached to the frame of conventional eyeglasses. In an example, an eating detector and/or an energy emitter can be incorporated into a necklace and/or pendant. In an example, an eating detector and/or an energy emitter can be incorporated into a headband. In an example, an eating detector and/or an energy emitter can be incorporated into a wearable patch or tattoo. In an example, an eating detector and/or an energy emitter can be incorporated into a wrist-worn device (e.g. smart watch, watch band, wrist band, or bracelet).

In this example, the energy emitter is a speaker. In this example, the speaker transmits encouraging and/or complimentary words to help modify the person's consumption in a desired manner. For example, some people respond to emotional stress by emotion-driven eating, which can make things worse in the long run. In an example, encouraging, calming, and/or complimentary words can help reduce the person's emotional stress, thereby reducing unhealthy emotion-driven food consumption. In this example, the speaker on the ear-worn device is saying—“You are a great person. You can handle this!” Such a system can literally provide “complimentary medicine.”

In an example, a system can further comprise one or more biometric sensors which are used to detect when the person is emotionally stressed and the system can provide a selected type of spoken-word stimulus to reduce unhealthy emotion-based food consumption when the person is emotionally stressed. In an example, a system can further comprise biometric sensors which track a person's blood pressure, pulse rate, and/or other biometric parameters to detect when the person is emotionally stressed, wherein the system can provide a selected pattern of spoken-word stimulus to reduce unhealthy emotion-based food consumption when the person is emotionally stressed. In an example, this spoken-word stimulus can be provided at selected anatomical locations on the surface of the person's head, neck, or torso. In an example, this system can provide real-time neurostimulation therapy to help a person to avoid unhealthy, emotion-drive food consumption.

In another example, an energy emitter can play music and/or a sequence of sounds or tones. In another example, this system can play a selected type or genre of music (or sound tones) when analysis of data from the eating detector indicates that a person is consuming a selected quantity or type of food. In another example, the selected music or sounds can be documented as helping to modify the person's food consumption in the past. In another example, this system can play soothing and/or calming music (or sound tones) when a person is consuming an unhealthy quantity or type of food in order to reduce the person's consumption of the unhealthy quantity or type of food. In another example, the tempo of music (or sound tone patterns) played can be selected to encourage the person to eat more slowly.

In another example, an energy emitter can be an electrical and/or electromagnetic energy emitter. In an example, an energy emitter can be an electrode which emits electrical and/or electromagnetic energy. In an example, an energy emitter can be an electrode which provides neurostimulation of the brain and/or peripheral nerves, wherein this neurostimulation modifies the person's food consumption. In an example, an energy emitter can be an electrode which provides neurostimulation of the brain and/or peripheral nerves, wherein this neurostimulation modifies the person's food consumption by reducing hunger.

In this example, the data processor which analyzes data from the eating detector and selects the pattern of energy (e.g. spoken word stimulus) to be emitted by an energy emitter is part of a wearable device (e.g. ear-worn device). In another example, a data processor which analyzes data from an eating detector and selects the pattern of energy to be emitted by an energy emitter can a remote data processor with which a local data processor (and data transmitter) can be in electronic communication. In an example, a remote data processor can be located in “the cloud” (a remote server) with which a local data processor is in electronic communication via the internet. In an example, this system can further comprise a wireless data transmitter, a wireless data receiver, and a power supply (e.g. battery).

In an example, this system can further comprise an electronic data file which includes historical information on food consumption and historical information on emitted energy patterns. This information is analyzed to identify which specific energy patterns (e.g. spoken words) have been most effective in modifying subsequent food consumption in a desired manner. In an example, this analysis of historical information can be used to help select which spoken words are selected to be transmitted by the system to modify which types of food consumption. In an example, a particular word phrase or expression may be most effective at reducing unhealthy food consumption. Example variations discussed elsewhere in this disclosure can also be applied to this example where relevant.

The following are example variations which can be applied, where relevant, to the examples shown in FIGS. 1 through 6. In an example, a wearable system and/or device for modifying a person's food consumption can comprise: (a) an eating detector which is worn by a person, wherein data from the eating detector is analyzed to identify food consumption by the person; (b) one or more energy emitters which are worn by the person, wherein one or more patterns of energy emitted from the one or more energy emitters are selected to change the person's food consumption in a desired manner (e.g. to reduce the person's consumption of an unhealthy quantity and/or type of food); and (c) a data processor, wherein data from the eating detector is analyzed in the data processor, and wherein selection of the one or more patterns of energy to be emitted from the one or more energy emitters is based in part on analysis of current data from the eating detector and/or based on identified historical associations between patterns of energy emitted from the one or more energy emitters and subsequent changes in the person's food consumption.

In an example, a method for modifying a person's food consumption can comprise: (a) analyzing data from an eating detector which is worn by a person to identify food consumption by the person; (b) selecting an energy pattern to be emitted by one or more energy emitters worn by the person, wherein the energy pattern is selected due to its past effectiveness in modifying the person's food consumption in a desired manner (e.g. reducing the person's consumption of an unhealthy quantity and/or type of food); and (c) emitting the energy pattern from the one or more energy emitters worn by the person.

In an example, one or more energy emitters can be speakers and/or ear buds which play music and/or sound tone patterns to modify a person's food consumption in a desired manner (e.g. to reduce a person's consumption of an unhealthy quantity or type of food). In an example, specific types of music and/or sound tone patterns can be selected based in part on analysis of current data from the eating detector and/or based on identified historical associations between emitted music and/or sound tone patterns and subsequent changes in the person's food consumption. In an example, various music and/or sound tone patterns can be selected based on one or more attributes selected from the group consisting of: genre; mood; tempo and/or beat; instrumentation; pitch; melody; loudness; popularity or prevalence; and lyrical content.

In another example, a system can further comprise one or more biometric sensors which measure biometric parameters which can be used to identify when the person is under emotional stress which is considered when the types of energy pattern is selected. In an example, these biometric parameters can be selected from the group consisting of: blood pressure; heart rate; heart rate variability; respiration rate; skin electroconductivity; skin moisture level; and EEG pattern. In an example, an energy pattern to be emitted can be selected as a multivariate function of biometric parameters and food consumption parameters.

In an example, an eating detector and/or energy emitter can be part of a device which is worn on, or within, a person's ear (e.g. ear bud, ear loop, or ear ring). In an example, an eating detector and/or energy emitter can be part of a device which is worn on a person's arm and/or wrist (e.g. smart watch, watch band, wrist band, or arm band). In an example, an eating detector and/or energy emitter can be part of a device which is worn on, or around, a person's neck (e.g. necklace, pendant, collar, or skin patch). In an example, an eating detector and/or energy emitter can be part of a device which is worn near a person's eyes (e.g. smart glasses, VR eyewear, and/or AR eyewear). In an example, an eating detector and/or energy emitter can be part of a device which is around a portion of a person's head (e.g. head band or hat).

In an example, a system can activate (e.g. trigger) an energy emitter to emit a pattern of energy which modifies a person's food consumption when a person first starts eating, based on data from an eating detector. In an example, a system can activate (e.g. trigger) an energy emitter to emit a pattern of energy when a person starts eating a particular type of food, based on data from an eating detector. In an example, a system can activate (e.g. trigger) an energy emitter to emit a pattern of energy when a person has cumulatively eaten a selected quantity of food, based on data from an eating detector. In an example, a system can activate (e.g. trigger) an energy emitter to emit a pattern of energy when a person has cumulatively eaten a selected quantity of food during a selected time interval, based on data from an eating detector.

In an example, a system can activate (e.g. trigger) an energy emitter to emit a pattern of energy based on multivariate analysis of: the person's emotional state based on data from biometric sensors; and the person's food consumption based on data from an eating detector. In an example, a system can activate (e.g. trigger) an energy emitter to emit a pattern of energy when a person starts emotion-driven eating as determined by analysis of: the person's emotional state based on data from biometric sensors; and the person's food consumption based on data from an eating detector.

In an example, data from a wearable sound-based eating detector can be used to detect food consumption by a person based on chewing and/or swallowing sounds. In an embodiment, an energy emitter can emit music and/or sound tones to modify a person's food consumption. In an example, data from an inertial motion unit (e.g. comprising an accelerometer and gyroscope) can be used to estimate the quantity of food consumed by a person by tracking the person's hand-to-mouth and wrist-rotation motions. In an AR/VR eyewear embodiment of this invention, multivariate analysis can be used to identify which types of virtual images (e.g. displayed in AR/VR eyewear) are most effective in reducing a person's consumption of unhealthy quantities and/or types of food.

In an example, data concerning specific energy patterns and subsequent changes in a person's food consumption can be recorded and analyzed over time to identify those energy patterns which are particularly effective in modifying the person's food consumption in a desire manner. In an example, the relationship between specific energy patterns and a person's food consumption can be analyzed and/or identified using multivariate analysis. In an example, the relationship between specific energy patterns and a person's food consumption can be analyzed and/or identified using Chi-Squared Analysis. In another example, the relationship between specific energy patterns and a person's food consumption can be analyzed and/or identified using Cluster Analysis (CA). In an example, the relationship between specific energy patterns and a person's food consumption can be analyzed and/or identified using correlation.

In an example, this system can be triggered by detection of food consumption (based on data from the eating detector) to provide musical feedback to modify a person's food consumption in a desired manner. In one embodiment, an eating detector can be an electroencephalographic (EEG) sensor. In an example, an eating detector can comprise both a motion sensor and a camera, wherein the camera is activated to record images when data from the motion sensor indicates (a high probability of) eating-related motions. In an example, an energy emitter can be attached to a near-eye device such as eyeglasses. In another example, an energy emitter can emit patterns of electrical energy which comprise neurostimulation. In an example, an energy emitter can provide a pattern of kinetic vibration with a decreasing speed of oscillations to encourage reduction of the speed of a person's hand-to-mouth eating motions and/or chewing motions.

In another example, data from an eating detector can be analyzed using Machine Learning (ML). In an example, data from an eating detector can be analyzed using Machine Learning (ML). In another example, data from an eating detector can be analyzed using a Markov Model (MM). In one embodiment, data from an eating detector can be analyzed using Maximum Likelihood Analysis (MLA). In one embodiment, data from an eating detector can be analyzed using a Moving Time Window (MTW). In an example, data from an eating detector can be analyzed using Multivariate Linear Regression (MLR).

In an example, images of the space in front of a person can be recorded by a front-facing wearable camera and analyzed to detect food consumption. In an example, one or more energy emitters can be embodied in one or two ear buds which emit isochronic tones which cause brainwave entrainment in order to modify a person's food consumption in a desired manner, in real time. In an example, the tempo of music played by a system can be chosen in part based on the tempo of a person's eating-related hand-to-mouth motions, wherein selection of a musical tempo which is slower than that of hand-to-motion motions can help to slow the person's hand-to-mouth motions and help the person to eat slower and/or less. In an example, this system can provide real-time feedback to modify a person's food consumption in a desired manner.

In another example, an eating detector can be embodied in a wrist-worn device such as a smart watch or wrist band. In one embodiment, an eating detector can comprise an optical detector with a light emitter and a light receiver, wherein the light receiver receives light from the light emitter after this light has been reflected by a person's body. In an example, an energy emitter can be an electrical energy emitter. In an example, an energy emitter can emit of pattern of neurostimulation which reduces a person's appetite. In another example, an energy emitter can play music.

In an example, data from an eating detector can be analyzed using Fuzzy Logic (FL). In an example, data from an eating detector can be analyzed using a Gaussian Model (GM). In one embodiment, data from an eating detector can be analyzed using Generalized Auto-Regressive Conditional Heteroscedasticity (GARCH) modeling. In another example, data from an eating detector can be analyzed using Independent Components Analysis (ICA). In an example, data from an eating detector can be analyzed using Inverse Dynamics Model (IDM).

In an example, images from a wearable camera can be analyzed to detect a person's food consumption. In an example, one or more energy emitters can be embodied in one or two ear buds which emit sound pulses and/or tones which cause brainwave entrainment in order to modify a person's food consumption in a desired manner, in real time. In an example, the relationship between specific energy patterns and a person's food consumption can be analyzed and/or identified using Random Forest Analysis (RFA). In an example, the relationship between specific energy patterns and a person's food consumption can be analyzed and/or identified using Support Vector Machine (SVM). In one embodiment, the relationship between specific energy patterns and a person's food consumption can be analyzed and/or identified using Time Series Analysis (TSA).

In an example, a system can provide musical stimulus and/or feedback to modify a person's food consumption in a desired manner. In an example, a wearable component of this system can include a data processor and also a data transmitter, a data receiver, and a power source (e.g. battery). In an example, an eating detector can be removably attached to (the sidepiece of) an eyeglasses frame. In an example, an energy emitter can be a speaker. In another example, an energy emitter can display virtual content in a person's field of view in AR or VE eyeglasses, in real time, wherein this virtual content helps to reduce the person's consumption of unhealthy quantities and/or types of food. In another example, an energy emitter can play music which has been associated with a reduction in quantity of food consumed by a person in the past. In an example, playing Weird Al's musical parody of “Bad” might do the trick.

In an example, data from a wearable sound-based eating detector can be used to estimate the quantity of food consumed by a person based on the number or types of chewing and/or swallowing sounds. In an example, data from an inertial motion unit can be used to estimate the quantity of food consumed by a person by tracking the person's throat movements. In another example, multivariate analysis of previous energy emission patterns and previous food consumption can be used to identify which energy emission patterns have the greatest effect on food consumption.

In an example, the relationship between specific energy patterns and a person's food consumption can be analyzed and/or identified using Discriminant Analysis (DA). In another example, the relationship between specific energy patterns and a person's food consumption can be analyzed and/or identified using Factor Analysis (FA). In one embodiment, the relationship between specific energy patterns and a person's food consumption can be analyzed and/or identified using Feature Vector Analysis (FVA).

In an example, this system can be triggered to emit an energy pattern (e.g. music, sounds, neurostimulation, vibrations, spoken word stimulus, images displayed) based on detection of food consumption (based on data from the eating detector). In an example, the energy pattern (e.g. music, sounds, neurostimulation, vibrations, spoken word stimulus, images displayed) which is most effective in modifying a person's food consumption can be identified based on analysis of past associations between energy patterns and food consumption. In an example, a pattern of energy emission can be adjusted based on analysis of historical data showing what type of pattern is most effective in modifying the person's food consumption in a desired manner.

In an example, an eating detector can be attached to a near-eye display device such as smart eyeglasses. In an example, an eating detector can comprise both an EMG sensor and a camera, wherein the camera is activated to record images when data from the EMG sensor indicates (a high probability of) eating-related jaw muscle movement. In an example, an energy emitter can be incorporated into an article of smart clothing (e.g. the collar of a smart shirt).

In an example, an energy emitter can emit patterns of sonic energy. In an example, an energy emitter can provide a selected pattern of kinetic vibration to reduce the speed of a person's food consumption. In one embodiment, data from an eating detector can be used to identify the types of food that a person consumes. In an example, multivariate analysis can be used to identify which types of energy patterns are most effective in reducing a person's consumption of unhealthy quantities and/or types of food. In an example, selection of the pattern of sound tones which a system plays to modify a person's food consumption can be based in part on a person's biometric parameters (e.g. blood pressure, heart rate, breathing pattern, and/or EEG pattern), especially if they collectively suggest that the person's food consumption is being triggered by the person's emotions or stress level.

In an example, this system can be embodied in a smart necklace. In an example, this system can provide visual feedback to reduce a person's consumption of unhealthy quantities and/or types of food. In an example, a wearable eating detector can be a wearable image-based detector such as a wearable camera. In an example, an eating detector can be removably attached to the sidepiece of an eyeglasses frame. In an example, an energy emitter can be a wrist-worn device such as a smart watch or wrist band. In one embodiment, an energy emitter can display virtual content next to food in the environment in a person's field of view in AR or VE eyeglasses. In an example, an energy emitter can play music which is associated with a reduction in eating speed for the person wearing the device.

In an example, data from a wearable sound-based eating detector can be used to estimate the quantity of food consumed by a person based on the quantity, frequency, amplitude, duration, and/or pitch of chewing and/or swallowing sounds. In an example, data from the eating detector can be analyzed to identify, in real time, when a person is eating. In an example, multivariate analysis of previous energy emission patterns and previous food consumption can be used to identify which energy emission patterns have a desired and/or selected effect on food consumption.

In another example, the relationship between specific energy patterns and a person's food consumption can be analyzed and/or identified using a Forward Dynamics Model (FDM). In an example, the relationship between specific energy patterns and a person's food consumption can be analyzed and/or identified using Fourier Transformation (FT). In another example, the relationship between specific energy patterns and a person's food consumption can be analyzed and/or identified using Fuzzy Logic (FL).

In an example, this system can emit energy when triggered by detection of food consumption (based on data from the eating detector) and provide visual feedback to reduce a person's consumption of unhealthy quantities and/or types of food. In one embodiment, a wearable eating detector can be an inertial motion unit. In an example, an eating detector can be worn on a person's head. In an example, an energy emitter can be activated and/or triggered to emit a pattern of energy which affects person's food consumption when an eating detector detects that the person has started eating. In another example, an energy emitter can emit a calming pattern (or color) of light to sooth a person and reduce emotionally-driven eating by that person. In an example, an energy emitter can play music which has been associated with a reduction in quantity of food consumed in the past for the person wearing the device.

In another example, data from a wearable sound-based eating detector can be used to estimate the quantity of food consumed by a person based on quantity, frequency, amplitude, duration, and/or pitch of dishware and utensil sounds. In an example, data from the eating detector can be analyzed to identify, in real time, the quantities and/or types of food which a person is consuming. In an example, multivariate analysis of previous energy emission patterns and previous food consumption can be used to identify which energy emission patterns are most effective at reducing consumption of unhealthy types and/or quantities of food.

In one embodiment, the relationship between specific energy patterns and a person's food consumption can be analyzed and/or identified using a Gaussian Model (GM). In an example, the relationship between specific energy patterns and a person's food consumption can be analyzed and/or identified using Generalized Auto-Regressive Conditional Heteroscedasticity (GARCH) Modeling. In an example, the relationship between specific energy patterns and a person's food consumption can be analyzed and/or identified using Independent Components Analysis (ICA).

In an example, this system can be triggered to emit energy by detection of food consumption (based on data from the eating detector) to provide tactile feedback to modify a person's food consumption in a desired manner. In an example, adjusting a pattern of energy emitted by an energy emitter can help to identify one or more patterns which are particularly effective in modifying a person's food consumption in a desired manner (e.g. reducing the person's consumption of an unhealthy quantity and/or type of food). In an example, an eating detector can be worn on a person's neck. In an example, an energy emitter can be activated and/or triggered to emit a pattern of energy which affects a person's food consumption when an eating detector detects that the person has already eaten a selected quantity of food during a selected period of time.

In an example, an energy emitter can emit a selected pattern of electrical energy which reduces a person's appetite. In an example, an energy emitter can play music which in the past has been associated with a subsequent reduction in eating speed. In an example, data from a wearable sound-based eating detector can be used to identify the type of food consumed by a person based on chewing and/or swallowing sounds. In another example, historical information concerning past food consumption based on data from the eating detector and historical information concerning past patterns of energy emission from the energy emitter can be jointly analyzed to identify a relationship between energy emission patterns and food consumption. In one embodiment, multivariate analysis of previous energy emission patterns and previous food consumption can be used to identify which energy emission patterns are most effective at increasing consumption of healthy types and/or quantities of food.

In an example, the relationship between specific energy patterns and a person's food consumption can be analyzed and/or identified using an Inverse Dynamics Model (IDM). In an example, the relationship between specific energy patterns and a person's food consumption can be analyzed and/or identified using a Kalman Filter (KF). In an example, the relationship between specific energy patterns and a person's food consumption can be analyzed and/or identified using Kernel Estimation (KE). In an example, the relationship between specific energy patterns and a person's food consumption can be analyzed and/or identified using Least Squares Estimation (LSE).

In an example, this system can be triggered by detection of food consumption (based on data from the eating detector) to provide tactile feedback to reduce a person's consumption of unhealthy quantities and/or types of food. In another example, a pattern of energy emissions can comprise a sequence and/or pattern of electromagnetic pulses. In an example, an eating detector can be incorporated into an article of smart clothing (e.g. the collar of a smart shirt). In another example, an energy emitter can be a loudspeaker which plays music, where a particular piece of music is selected to be played which was particularly effective in modifying the person's food consumption in a desired manner in the past. In one embodiment, an energy emitter can be removably attached to an eyeglasses frame.

In an example, an energy emitter can emit sound tones with a pattern of decreasing tempo to promote a reduction in eating speed. In an example, changing a person's food consumption in a desired manner can comprise reducing the person's consumption of food above a selected quantity in a selected period of time. In an example, data from an inertial motion unit can be used to detect food consumption by tracking a person's throat movements. In an example, multivariate analysis can be used to identify which types of electrical pulses are most effective in reducing a person's consumption of unhealthy quantities and/or types of food in particular environmental circumstances and/or given particular biometric parameter values. In an example, the pattern of energy selected to modify a person's food consumption can be based in part on biometric parameters (e.g. blood pressure, heart rate, breathing pattern, and/or EEG pattern) which suggest that the person's food consumption is being triggered by the person's emotions or stress level.

In another example, this system can be triggered by detection of food consumption (based on data from the eating detector) to provide real-time feedback to reduce a person's consumption of unhealthy quantities and/or types of food. In an example, adjusting the pitch, tempo, and/or pattern of tones played by (the energy emitter of) the device can help to identify tones which are particularly effective in modifying a person's food consumption in a desired manner (e.g. reducing the person's consumption of an unhealthy quantity and/or type of food). In another example, an eating detector can be worn on a person's torso. In one embodiment, an energy emitter can be activated and/or triggered to emit a pattern of energy which affects a person's food consumption when an eating detector detects that the person has already eaten a selected quantity of food at one sitting.

In another example, an energy emitter can emit a selected pattern of sound tones. In another example, an energy emitter can play music which in the past has been associated with a subsequent reduction in quantity of food consumed. In an example, data from a wearable sound-based eating detector can be used to identify the type of food consumed by a person based on quantity, frequency, amplitude, duration, and/or pitch of chewing and/or swallowing sounds. In one embodiment, historical information concerning past food consumption based on data from the eating detector and historical information concerning past patterns of energy emission from the energy emitter can be jointly analyzed to identify which energy emission patterns have the greatest effect on food consumption.

In an example, engaging a person who is eating can help to modify their food consumption in a desirable manner. In an example, a person who engages in a conversation while eating may consume calories less quickly and have their hunger satiated with less food. In an example, this system can initiate conversation with a person when the person's food consumption is approaching an unhealthy quantity. In an example, this system can comprise a speaker which transmits spoken word prompts, a microphone which records a person's spoken word responses, and an AI-powered chat function to engage an eating person in conversation.

In an example, multivariate analysis of previous energy emission patterns and previous food consumption can be used to identify a relationship between energy emission patterns and food consumption and this relationship can be used to adjust a pattern of energy emission to modify food consumption in a desired manner. In one embodiment, the relationship between specific energy patterns and a person's food consumption can be analyzed and/or identified using Linear Regression (LR). In another example, the relationship between specific energy patterns and a person's food consumption can be analyzed and/or identified using Logistic Regression (LR). In an example, the relationship between specific energy patterns and a person's food consumption can be analyzed and/or identified using Logit Analysis (LA). In another example, the relationship between specific energy patterns and a person's food consumption can be analyzed and/or identified using Logit Model (LM).

In an example, this system can comprise a local data processor which is part of a wearable component and a remote data processor with which the local data processor is in electronic communication (e.g. wireless communication). In an example, adjusting the types and/or pieces of music played by (the energy emitter of) the device can help to identify one or more types and/or pieces of music which are particularly effective in modifying a person's food consumption in a desired manner (e.g. reducing the person's consumption of an unhealthy quantity and/or type of food). In an example, an eating detector can be worn on a person's wrist and/or arm. In an example, an energy emitter can be activated and/or triggered to emit a pattern of energy which affects a person's food consumption when an eating detector detects that the person is eating a type of food which has been identified as unhealthy.

In another example, an energy emitter can emit kinetic energy. In one embodiment, an energy emitter can play music which in the past has been associated with a subsequent reduction in eating speed for the person wearing the device. In another example, data from a wearable sound-based eating detector can be used to identify the type of food consumed by a person based on quantity, frequency, amplitude, duration, and/or pitch of dishware and utensil sounds. In an example, historical information concerning past food consumption based on data from the eating detector and historical information concerning past patterns of energy emission from the energy emitter can be jointly analyzed to identify which energy emission patterns have a desired and/or selected effect on food consumption.

In an example, a sonic energy emitter can emit sound patterns based on a person's chewing motions as detected by an eating detector. In an example, the frequency of sound tones can be based on the frequency of chewing motions detected by an eating detector. In an example, the frequency of sound tones can be slightly less than the frequency of chewing motions, thereby inducing (e.g. entraining) the person to chew more slowly.

In another example, multivariate analysis of previous energy emission patterns and previous food consumption can be used to identify which energy emission patterns have the greatest effect on food consumption and this relationship can be used to adjust a pattern of energy emission to modify food consumption in a desired manner. In an example, the relationship between specific energy patterns and a person's food consumption can be analyzed and/or identified using Machine Learning (ML). In an example, the relationship between specific energy patterns and a person's food consumption can be analyzed and/or identified using Machine Learning ML). In an example, the relationship between specific energy patterns and a person's food consumption can be analyzed and/or identified using Markov Model (MM).

In an example, this system can function as a provider of real-time music therapy to modify a person's food consumption, in real time, in a desired manner. In one embodiment, an eating detector can be a near-eye device such as eyeglasses. In another example, an eating detector can comprise a spectroscopic sensor. In an example, an energy emitter can be activated to emit a pattern of energy which affects a person's food consumption when biometric parameters suggest that the person's food consumption is being triggered by the person's emotions or stress level. In another example, an energy emitter can emit light energy. In an example, an energy emitter can play music which in the past has been associated with a subsequent reduction in quantity of food consumed for the person wearing the device.

In an example, a kinetic energy emitter can vibrate based on a person's chewing motions as detected by an eating detector. In an example, the frequency of vibrations can be based on the frequency of chewing motions detected by an eating detector. In an example, the frequency of vibrations can be slightly less than the frequency of chewing motions, thereby inducing (e.g. entraining) the person to chew more slowly.

In an example, data from an eating detector can be analyzed using Analysis of Variance (ANOVA). In an example, data from an eating detector can be analyzed using Artificial Intelligence (AI). In an example, data from an eating detector can be analyzed using Artificial Neural Network (ANN). In one embodiment, data from an eating detector can be analyzed using Auto Regression (AR). In an example, data from an eating detector can be analyzed using Back Propagation Network (BPN). In an example, data from an eating detector can be analyzed using Bayesian Analysis. In another example, data from an eating detector can be analyzed using Chi-Squared Analysis.

In an example, historical information concerning past food consumption based on data from the eating detector and historical information concerning past patterns of energy emission from the energy emitter can be jointly analyzed to identify which energy emission patterns are most effective at reducing consumption of unhealthy types and/or quantities of food. In another example, multivariate analysis of previous energy emission patterns and previous food consumption can be used to identify which energy emission patterns have a desired and/or selected effect on food consumption and this relationship can be used to adjust a pattern of energy emission to modify food consumption in a desired manner.

In an example, the relationship between specific energy patterns and a person's food consumption can be analyzed and/or identified using Maximum Likelihood Analysis (MLA). In another example, the relationship between specific energy patterns and a person's food consumption can be analyzed and/or identified using Moving Time Window (MTW). In an example, the relationship between specific energy patterns and a person's food consumption can be analyzed and/or identified using Multivariate Linear Regression (MLR). In another example, this system can function as a provider of real-time music therapy to reduce a person's consumption of unhealthy quantities and/or type of food, in real time.

In one embodiment, an eating detector can be an electromyographic (EMG) sensor. In an example, an eating detector can comprise both a motion sensor and a camera, wherein the camera is activated to record images when data from the motion sensor indicates (a high probability of) eating-related jaw motion. In an example, an energy emitter can be attached to a wrist-worn device such as a watch or wrist band. In an example, an energy emitter can emit patterns of electrical energy which comprise neurostimulation of one or more nerves. In an example, an energy emitter can provide a pattern of sonic vibration with a decreasing speed of oscillations to encourage reduction of the speed of a person's hand-to-mouth eating motions and/or chewing motions.

In an example, a light energy emitter can display pulses of light based on a person's chewing motions as detected by an eating detector. In an example, the frequency of light pulses can be based on the frequency of chewing motions detected by an eating detector. In an example, the frequency of light pulses can be slightly less than the frequency of chewing motions, thereby inducing (e.g. entraining) the person to chew more slowly.

In an example, data from an eating detector can be analyzed using Probit Analysis (PA). In one embodiment, data from an eating detector can be analyzed using Random Forest Analysis (RFA). In another example, data from an eating detector can be analyzed using Support Vector Machine (SVM). In an example, data from an eating detector can be analyzed using Time Series Analysis (TSA). In another example, the relationship between specific energy patterns and a person's food consumption can be analyzed and/or identified using Analysis of Variance (ANOVA).

In an example, images recorded by a wearable camera can be analyzed to identify types and quantities of nearby food. In another example, one or more energy emitters can emit isochronic tones which cause brainwave entrainment in order to modify a person's food consumption in a desired manner, in real time. In an example, the tempo of music played by the system can be chosen to first be in sync with the tempo of a person's eating-related hand-to-mouth motions, but then be gradually slowed to help slow the person's hand-to-mouth motions. In another example, this system can provide tactile feedback to modify a person's food consumption in a desired manner. In an example, an eating detector can be an electroencephalographic (EEG) sensor which monitors a person's brainwave activity to identify brainwave patterns which indicate food consumption.

In an example, an eating detector can comprise both a motion sensor and a camera, wherein the camera is activated to record images when data from the motion sensor indicates (a high probability of) eating-related hand motion. In an example, an energy emitter can be attached to a neck-worn device such as a necklace or pendant. In one embodiment, an energy emitter can emit patterns of electrical energy which comprise neurostimulation of the brain. In an example, an energy emitter can provide a pattern of sonic vibration with a decreasing speed of oscillations to reduce the speed of a person's food consumption.

In another example, data from an eating detector can be analyzed using Multivariate Logit (ML). In one embodiment, data from an eating detector can be analyzed using Non-Linear Programming (NLP). In another example, data from an eating detector can be analyzed using Non-Linear Regression (NLR). In an example, data from an eating detector can be analyzed using Pattern Recognition Engine. In another example, data from an eating detector can be analyzed using Polynomial Function Estimation (PFE). In one embodiment, data from an eating detector can be analyzed using Principal Components Analysis (PCA). In an example, images recorded by a wearable camera can be analyzed to identify nearby food. In an example, one or more energy emitters can emit binaural beats which cause brainwave entrainment in order to modify a person's food consumption in a desired manner, in real time.

In an example, the tempo of music played by the system can be chosen in part based on the tempo of a person's jaw (e.g. chewing) movements, wherein selection of a musical tempo which is slower than that of jaw movements can help to slow the person's jaw movements and help the person to eat slower and/or less. In an example, this system can provide real-time feedback to reduce a person's consumption of unhealthy quantities and/or types of food. In another example, a pattern of energy emissions can comprise a sequence and/or pattern of electrical pulses. In an example, an eating detector can be part of a near-eye device such as eyeglasses. In another example, an energy emitter can be a loudspeaker which plays music, where a particular piece of music is selected to be played which was particularly effective in reducing emotion-driven food consumption by the person in the past.

In an example, an energy emitter can be removably attached to the sidepiece of an eyeglasses frame. In another example, an energy emitter can emit sound tones with a selected pitch and/or frequency. In an example, data from a wearable eating detector can comprise a sound-based sensor such as a microphone. In one embodiment, data from an inertial motion unit can be used to estimate the quantity of food consumed by a person by tracking the person's hand motions. In an example, multivariate analysis can be used to identify which types of electrical pulses are most effective in reducing a person's consumption of unhealthy quantities and/or types of food currently detected by the eating detector.

In an example, the pitch of music and/or tones played by a device can be varied to help identify what pitch is most effective in modifying a person's food consumption in a desired manner (e.g. reducing consumption of unhealthy quantities or types of food). When a particularly effective pitch is identified, then this pitch can be used for paying music and/or tones in the future for effective food consumption modification. In an example, this system can be triggered by detection of food consumption (based on data from the eating detector) to provide auditory feedback to modify a person's food consumption in a desired manner. In an example, a system can further comprise one or more biometric sensors which measure biometric parameters which can be used to identify when the person is under emotional stress which may cause unhealthy eating.

In another example, an eating detector can be part of a neck-worn device such as a necklace or pendant. In an example, an energy emitter can be a near-eye device such as eyeglasses. In another example, an energy emitter can display images in a person's field of view in AR or VE eyeglasses. In an example, an energy emitter can emit sound tones with a selected frequency progression. In another example, data from a wearable sound-based eating detector can be used to detect food consumption by a person based on chewing and/or swallowing sounds. In one embodiment, data from an inertial motion unit can be used to estimate the quantity of food consumed by a person by tracking the person's hand-to-mouth motions. In an example, multivariate analysis can be used to identify which types of virtual images (e.g. displayed in AR/VR eyewear) are most effective in reducing a person's consumption of unhealthy quantities and/or types of food in particular environmental circumstances and/or given particular biometric parameter values.

In an example, the relationship between specific energy patterns and a person's food consumption can be analyzed and/or identified using Artificial Intelligence (AI). In an example, the relationship between specific energy patterns and a person's food consumption can be analyzed and/or identified using Artificial Neural Network (ANN). In an example, the relationship between specific energy patterns and a person's food consumption can be analyzed and/or identified using Auto Regression (AR). In an example, the relationship between specific energy patterns and a person's food consumption can be analyzed and/or identified using Back Propagation Network (BPN). In another example, the relationship between specific energy patterns and a person's food consumption can be analyzed and/or identified using Bayesian Analysis.

In an example, this system can be triggered by detection of food consumption (based on data from the eating detector) to provide auditory feedback to reduce a person's consumption of unhealthy quantities and/or types of food. In another example, a data processor can be part of a wearable component of this system. In one embodiment, an eating detector can be an electromyographic (EMG) sensor which tracks activity of the muscles which move a person's jaw. In another example, an eating detector can comprise both a non-imaging optical sensor and a camera, wherein the camera is activated to record images when data from the non-imaging optical sensor indicates (a high probability of) eating-related motion. In an example, an energy emitter can be attached to an ear-worn device such as an earbud. In an example, an energy emitter can emit patterns of electrical energy which comprise neurostimulation of the Vegas nerve. In an example, an energy emitter can provide a selected pattern of vibration to modify a person's food consumption.

In an example, data from an eating detector can be used to detect when a person consumes food. In an example, images recorded by a wearable camera can be analyzed to identify types, quantities, and changes in quantities of nearby food in order to estimate the types and quantities of food consumed by a person. In an example, one or more energy emitters can emit sound pulses and/or tones which cause brainwave entrainment in order to modify a person's food consumption in a desired manner, in real time. In another example, this system can be embodied in a smart button, pin, and/or pendant. In one embodiment, this system can provide tactile feedback to reduce a person's consumption of unhealthy quantities and/or types of food. In another example, a pattern of energy emissions can comprise a musical composition. In an example, an eating detector can be attached to a neck-worn device such as a necklace or pendant. In another example, an energy emitter can be a digital display. In one embodiment, an energy emitter can be part of a near-eye device such as eyeglasses.

In an example, an energy emitter can emit sonic energy. In one embodiment, an energy emitter can be a loudspeaker which produces, transmits, and/or broadcasts spoken words. In another example, an energy emitter can be a loudspeaker which produces, transmits, and/or broadcasts positive, encouraging, and/or complimentary words to the person wearing the device. In an example, an energy emitter can be a loudspeaker which produces, transmits, and/or broadcasts positive, encouraging, and/or complimentary words to a person wearing a device when the device detects that the person is consuming food in an unhealthy manner. In another example, an energy emitter can be a loudspeaker which produces, transmits, and/or broadcasts positive, encouraging, and/or complimentary words to a person wearing a device when the device detects that the person is consuming an unhealthy quantity and/or type of food. In an example, the device can say complimentary things to encourage the person to reduce emotion-driven eating. In another example, the device can say complimentary things such as—“You are a wonderful person” or “Relax, you got this” or “You will be fine.” In an example, a device can use complimentary medicine (pun intended) to encourage better eating habits.

In another example, an energy emitter can provide a selected pattern of sonic vibration to reduce the speed of a person's food consumption. In an example, data from an inertial motion unit can be used to detect food consumption by tracking a person's hand motions. In another example, multivariate analysis can be used to identify which types of music and/or sound tone patterns are most effective in reducing a person's consumption of unhealthy quantities and/or types of food in particular environmental circumstances and/or given particular biometric parameter values. In an example, selection of the type or piece music which a system plays to modify a person's food consumption can be based in part on a person's biometric parameters (e.g. blood pressure, heart rate, breathing pattern, and/or EEG pattern), especially if they collectively suggest that the person's food consumption is being triggered by the person's emotions or stress level.

In an example, this system can be embodied in a smart shirt and/or collar. In an example, this system can use adaptive machine learning (such as a neural network or artificial intelligence) to learn what energy patterns are most effective at modifying a person's food consumption in a desired manner. In an example, a system can further comprise one or more biometric sensors which measure biometric parameters which can be used to identify when the person is under emotional stress and considered in the selection of soothing and/or calming energy patterns (e.g. soothing music or tones). In one embodiment, an eating detector can be part of an ear-worn device such as an earbud. In an example, an energy emitter can be a neurostimulator. In an example, an energy emitter can display virtual content in a person's field of view in AR or VE eyeglasses, in real time, wherein this virtual content helps to modify the person's food consumption in a desired manner.

In an example, an energy emitter can play music which has been associated with a reduction in eating speed in the past. In another example, data from a wearable sound-based eating detector can be used to detect food consumption by a person based on the sounds of dishware and utensils. In an example, data from an inertial motion unit can be used to estimate the quantity of food consumed by a person by tracking the person's jaw motions. In another example, multivariate analysis of previous energy emission patterns and previous food consumption can be used to identify a relationship between energy emission patterns and food consumption.

In an example, the relationship between specific energy patterns and a person's food consumption can be analyzed and/or identified using Covariance Analysis (CA). In another example, the relationship between specific energy patterns and a person's food consumption can be analyzed and/or identified using Decision Tree Analysis (DTA). In an example, the relationship between specific energy patterns and a person's food consumption can be analyzed and/or identified using Discrete Fourier Transform (DFT). In another example, this system can be triggered by detection of food consumption (based on data from the eating detector) to provide musical feedback to reduce a person's consumption of unhealthy quantities and/or types of food.

In an example, a pattern of energy emissions can comprise a sequence and/or pattern of sound tones. In one embodiment, an eating detector can be attached to a wrist-worn device such as a watch or wrist band. In an example, an energy emitter can be a display which is part of smart eyeglasses. In an example, an energy emitter can be part of a neck-worn device such as a necklace or pendant. In an example, an energy emitter can emit sound energy. In an example, an energy emitter can vibrate. In an example, data from an inertial motion unit can be used to detect food consumption by tracking a person's hand-to-mouth motions.

In an example, multivariate analysis can be used to identify which types of music and/or sound tone patterns are most effective in reducing a person's consumption of unhealthy quantities and/or types of food currently detected by the eating detector. In another example, showing a particular image in a person's field of vision, in real time, can help to reduce the person's consumption of an unhealthy quantity and/or type of food. In an example, this system can be embodied in a smart watch, watch band, and/or wrist band. In another example, this system can use adaptive machine learning (such as a neural network or artificial intelligence) to learn what energy patterns are most effective at reducing a person's consumption of unhealthy quantities and/or types of food, in real time. In one embodiment, a pattern of energy emissions can comprise a sequence and/or pattern of vibrations. In another example, an eating detector can be incorporated into a wearable button. In an example, an energy emitter can be a Light Emitting Diode (LED).

In an example, an energy emitter can be part of an ear-worn device such as an earbud. In an example, an energy emitter can emit sound tones with a pattern of decreasing tempo. In an example, changing a person's food consumption in a desired manner can comprise reducing the person's consumption of an unhealthy quantity and/or type of food. In an example, data from an inertial motion unit can be used to detect food consumption by tracking a person's jaw motions. In an example, multivariate analysis can be used to identify which types of neurostimulation are most effective in reducing a person's consumption of unhealthy quantities and/or types of food currently detected by the eating detector.

In one embodiment, the mood or style e.g. upbeat, sad, dramatic, calming) of music played by a device can be varied to help identify what mood or style is most effective in modifying a person's food consumption in a desired manner (e.g. reducing consumption of unhealthy quantities or types of food). When a particularly effective mood or style is identified, then this mood or style can be used for paying music and/or tones in the future for effective food consumption modification. In an example, this system can be triggered by detection of food consumption (based on data from the eating detector) to provide real-time feedback to modify a person's food consumption in a desired manner. Wherein data from the eating detector and data concerning energy emission patterns are analyzed to identify correlations between selected types of patterns and eating patterns, and these correlations are used to adjust energy patterns in the future. In another example, an eating detector can be an ear-worn device such as an earbud.

In an example, an eating detector can comprise both a microphone and a camera, wherein the camera is activated to record images when data from the microphone indicates (a high probability of) eating-related sounds. In another example, an energy emitter can be an electromagnetic energy emitter. In an example, an energy emitter can emit patterns of electrical energy. In another example, an energy emitter can provide a pattern of kinetic vibration with a decreasing speed of oscillations to reduce the speed of a person's food consumption.

In an example, data from an eating detector can be analyzed using Kalman Filter (KF). In an example, data from an eating detector can be analyzed using Kernel Estimation (KE). In one embodiment, data from an eating detector can be analyzed using Least Squares Estimation (LSE). In an example, data from an eating detector can be analyzed using Linear Regression (LR). In one embodiment, data from an eating detector can be analyzed using Logistic Regression (LR). In an example, data from an eating detector can be analyzed using Logit Analysis (LA). In one embodiment, data from an eating detector can be analyzed using Logit Model (LM).

In another example, images of a person's hands which are recorded by a wearable camera can be analyzed to detect the person's food consumption. In an example, one or more energy emitters can be embodied in one or two ear buds which emit binaural beats which cause brainwave entrainment in order to modify a person's food consumption in a desired manner, in real time. In another example, the tempo of music and/or tones played by a device can be varied to help identify what tempo is most effective in modifying a person's food consumption in a desired manner (e.g. reducing consumption of unhealthy quantities or types of food). When a particularly effective tempo is identified, then this tempo can be used for paying music and/or tones in the future for effective food consumption modification. In one embodiment, this system can provide musical feedback to reduce a person's consumption of unhealthy quantities and/or types of food.

In an example, a system can provide biofeedback through an energy emitter when a person first starts to consume food, as detected by the eating detector. In an example, a system can provide electrodermal biofeedback through an energy emitter when a person first starts to consume food, as detected by the eating detector. In an example, a system can provide heart rate variability biofeedback through an energy emitter when a person first starts to consume food, as detected by the eating detector. In an example, a system can provide diaphragmatic breathing biofeedback through an energy emitter when a person first starts to consume food, as detected by the eating detector.

In an example, a system can provide biofeedback through an energy emitter when a person's food consumption quantity approaches a selected (e.g. undesirable) quantity. In an example, a system can provide electrodermal biofeedback through an energy emitter when a person's food consumption quantity approaches a selected (e.g. undesirable) quantity. In an example, a system can provide heart rate variability biofeedback through an energy emitter when a person's food consumption quantity approaches a selected (e.g. undesirable) quantity. In an example, a system can provide diaphragmatic breathing biofeedback through an energy emitter when a person's food consumption quantity approaches a selected (e.g. undesirable) quantity.

In an example, a system can include an EEG sensor and can play sound patterns based on patterns in the person's brainwaves. In an example, such sound patterns can comprise neurofeedback. In an example, such sound patterns can comprise neurofeedback which can help a person to modify the quantity and/or types of food which they consume. In an example, a system can provide EEG training neurofeedback when a person's first starts to consume food. In an example, a system can provide EEG training neurofeedback when a person's food consumption quantity approaches a selected (e.g. undesirable) quantity.

In another example, an eating detector can be an EEG sensor which is part of, or attached to, the frame of eyeglasses. In an example, an eating detector can be an electromyographic (EMG) sensor which tracks activity of the muscles which move a person's arm and hand. In one embodiment, an eating detector can comprise both a non-imaging optical sensor and a camera, wherein the camera is activated to record images when data from the non-imaging optical sensor indicates (a high probability of) eating-related jaw muscle movement. In an example, an energy emitter can be incorporated into a wearable button. In an example, an energy emitter can emit patterns of electromagnetic energy. In an example, an energy emitter can provide a selected pattern of vibration to reduce a person's consumption of an unhealthy quantity and/or type of food.

In an example, data from an eating detector can be used to estimate the amount of food that a person consumes. In an example, multivariate analysis can be used to identify which types of energy patterns are most effective in reducing a person's consumption of unhealthy quantities and/or types of food in particular environmental circumstances and/or given particular biometric parameter values. In another example, playing a particular type, genre, and/or piece of music in a person's hearing, in real time, can help to reduce the person's consumption of an unhealthy quantity and/or type of food. In an example, this system can be embodied in a smart earring or ear bud.

In another example, this system can provide visual feedback to modify a person's food consumption in a desired manner. In one embodiment, an eating detector can be a non-imaging optical detector. In another example, an eating detector can be a non-imaging optical detector which comprises a light emitter which emits infrared light beams toward a person's jaw area and a light receiver which receives those light beams after they have been reflected from the person's jaw area, wherein changes in the light beams caused by jaw movement are analyzed to detect food consumption. In an example, an eating detector can comprise an optical detector with a light emitter and a light receiver, wherein the light receiver receives light from the light emitter after this light has been reflected by food. In an example, an energy emitter can be an ear-worn device such as an earbud. In an example, an energy emitter can emit music.

In an example, an energy emitter can play music with one or more selected characteristics and/or parameters selected from the group consisting of: content of lyrics, dissonance, dynamics, loudness, and/or softness, harmony, instrumental and/or vocal, instruments used, language of vocals, length and/or duration, melody, musical genre, pitch, popularity and/or common recognition, rhythm type, simplicity vs. complexity, spatial dynamics, tempo and/or beat, texture, timbre, and type of mood induced. In an example, data from an eating detector can be analyzed using Factor Analysis (FA). In one embodiment, data from an eating detector can be analyzed using Feature Vector Analysis (FVA). In an example, data from an eating detector can be analyzed using Forward Dynamics Model (FDM). In an example, data from an eating detector can be analyzed using Fourier Transformation (FT).

In an example, historical information concerning the effects of specific patterns of energy emission on a person's food consumption in the past is analyzed to select those patterns of energy emission which are used to modify the person's food consumption in real time. In another example, multivariate analysis of previous energy emission patterns and previous food consumption can be used to identify which energy emission patterns are most effective at increasing consumption of healthy types and/or quantities of food and this relationship can be used to adjust a pattern of energy emission to modify food consumption in a desired manner.

In an example, the relationship between specific energy patterns and a person's food consumption can be analyzed and/or identified using Polynomial Function Estimation (PFE). In another example, the relationship between specific energy patterns and a person's food consumption can be analyzed and/or identified using Principal Components Analysis (PCA). In an example, the relationship between specific energy patterns and a person's food consumption can be analyzed and/or identified using Probit Analysis (PA).

In another example, this system can provide auditory feedback to reduce a person's consumption of unhealthy quantities and/or types of food. In one embodiment, a pattern of energy emissions can comprise a sequence and/or pattern of light flashes. In an example, an eating detector can be attached to an ear-worn device such as an earbud. In one embodiment, an energy emitter can be a display which is part of AR or VR eyeglasses. In an example, an energy emitter can be part of a wrist-worn device such as a smart watch or wrist band. In another example, an energy emitter can emit sound tones with a pattern of decreasing frequency.

In an example, an inertial motion unit can comprise at least one of an accelerometer, a gyroscope, and a magnometer. In another example, data from an inertial motion unit can be used to detect food consumption by tracking a person's hand-to-mouth and wrist-rotation motions. In an example, multivariate analysis can be used to identify which types of neurostimulation are most effective in reducing a person's consumption of unhealthy quantities and/or types of food in particular environmental circumstances and/or given particular biometric parameter values. In an example, the genre of music played by a device can be varied to help identify what genre is most effective in modifying a person's food consumption in a desired manner (e.g. reducing consumption of unhealthy quantities or types of food). When a particularly effective genre is identified, then this genre can be used for paying music and/or tones in the future for effective food consumption modification. In an example, this system can be embodied in smart eyeglasses

In one embodiment, unhealthy food consumption may be driven by emotion and/or stress. In another example, soothing music and/or sounds may help to relieve stress and help reduce unhealthy food consumption which is driven by emotion and/or stress. In an example, particular pieces of music and/or sound patterns which have been identified as effectively reducing unhealthy food consumption in the past can be played to help reduce unhealthy food consumption in real time. In an example, when an eating detector identifies that a person is consuming an unhealthy quantity and/or type of food, then the device can pay soothing music and/or sounds to help the person reduce or stop this unhealthy consumption. In an example, music and/or sounds which are particularly effective can be identified by analysis of a historical relationship between music and/or sounds played by the device and food consumption.

In an example, an eating detector can be a neck-worn device such as a necklace or pendant. In another example, an eating detector can comprise an optical detector with a light emitter and a light receiver. In an example, an energy emitter can be activated to emit a pattern of energy which affects a person's food consumption when biometric parameters (e.g. blood pressure, heart rate, breathing pattern, and/or EEG pattern) suggest that the person's food consumption is being triggered by the person's emotions or stress level. In one embodiment, an energy emitter can emit light which provides phototherapy. In one embodiment, an energy emitter can play music with a selected (e.g. slow) tempo to encourage reduction of the speed of a person's hand-to-mouth eating motions and/or chewing motions.

In an example, data from an eating detector can be analyzed using Cluster Analysis (CA). In an example, data from an eating detector can be analyzed using Correlation. In an example, data from an eating detector can be analyzed using Covariance Analysis (CA). In an example, data from an eating detector can be analyzed using Decision Tree Analysis (DTA). In an example, data from an eating detector can be analyzed using Discrete Fourier Transform (DFT). In an example, data from an eating detector can be analyzed using Discriminant Analysis (DA).

In an example, historical information concerning past food consumption based on data from the eating detector and historical information concerning past patterns of energy emission from the energy emitter can be jointly analyzed to identify which energy emission patterns are most effective at increasing consumption of healthy types and/or quantities of food. In an example, multivariate analysis of previous energy emission patterns and previous food consumption can be used to identify which energy emission patterns are most effective at reducing consumption of unhealthy types and/or quantities of food and this relationship can be used to adjust a pattern of energy emission to modify food consumption in a desired manner. In an example, this system can provide auditory feedback to modify a person's food consumption in a desired manner.

In an example, the relationship between specific energy patterns and a person's food consumption can be analyzed and/or identified using Multivariate Logit (ML). In an example, the relationship between specific energy patterns and a person's food consumption can be analyzed and/or identified using Non-Linear Programming (NLP). In an example, the relationship between specific energy patterns and a person's food consumption can be analyzed and/or identified using Non-Linear Regression (NLR). In an example, the relationship between specific energy patterns and a person's food consumption can be analyzed and/or identified using a Pattern Recognition Engine.

I claim:

1. A method for reducing a person's consumption of unhealthy food comprising:
receiving images from at least one camera on at least one wearable device worn by a person;
analyzing the images to detect types and/or quantities of nearby food; and
providing haptic and/or tactile sensations for the person via at least one haptic and/or tactile component on the one or more wearable devices when analysis of the images detects unhealthy types and/or quantities of food near the person.

2. A method for reducing a person's consumption of unhealthy food comprising:
receiving data from at least one food consumption sensor on at least one wearable device worn by a person;
analyzing the data to detect types and/or quantities of food consumed by the person; and
providing haptic and/or tactile sensations for the person via at least one haptic and/or tactile component on the one or more wearable devices when analysis of the data detects consumption of unhealthy types and/or quantities of food by the person.

3. A system for reducing a person's consumption of unhealthy food comprising:
at least one wearable device worn by a person;
at least one camera on the at least one wearable device, wherein the camera captures images of nearby food, and wherein these images are analyzed to detect types and/or quantities of the nearby food; and
at least one haptic and/or tactile component on the one or more wearable devices, wherein the haptic component provides haptic sensations for the person when analysis of the images detects unhealthy types and/or quantities of food in the nearby food.
